(19) United States
(12) Patent Application Publication
(10) Pub. No.: US 2022/0389019 A1
(43) Pub. Date: Dec. 8, 2022

(54) TrkB POSITIVE ALLOSTERIC MODULATORS

(71) Applicants: Université de Strasbourg, Strasbourg (FR); Centre National de la Recherche Scientifique, Paris (FR); INSERM - Institut National de la Santé et de la Recherche Médicale, Paris (FR); Université Grenoble Alpes, Saint-Martin-d'Hères (FR)

(72) Inventors: Didier ROGNAN, Bernardswiller (FR); Martine SCHMITT, Strasbourg (FR); Maxime CAZORLA, Grenoble (FR)

(21) Appl. No.: 17/633,617

(22) PCT Filed: Aug. 7, 2020
(86) PCT No.: PCT/EP2020/072233

§ 371 Date: Feb. 8, 2022

(30) Foreign Application Priority Data
Aug. 8, 2019 (EP) 19315090.1

(51) Int. Cl. C07D 487/04; A61P 25/28

(52) U.S. Cl. CPC C07D 487/04; A61P 25/28

(57) ABSTRACT

The present invention relates to the field of pharmaceutical composition comprising “LIT-TB” derivatives. More particularly it relates to “LIT-TB” derivatives for use in the treatment of neurodegenerative diseases, and more particularly in the treatment of Huntington's disease. The invention also relates to the “LIT-TB” derivatives and preparation thereof.

### TECHNICAL FIELD

The present invention relates to the field of pharmaceutical composition comprising “LIT-TB” derivatives. More particularly it relates to “LIT-TB” derivatives for use in the treatment of neurodegenerative diseases, and more particularly in the treatment of Huntington's disease. The invention also relates to the “LIT-TB” derivatives and preparation thereof.

In the description below, references between [ ] refer to the list of references at the end of the examples.

### TECHNICAL BACKGROUND

Huntington's disease is an inherited disease that causes the progressive breakdown (degeneration) of nerve cells in the brain. Huntington's disease has a broad impact on a person's functional abilities and usually results in movement, thinking (cognitive) and psychiatric disorders.

Huntington's Disease (HD) is a rare, autosomal-dominant, neurodegenerative disorder, characterized by impaired motor control, cognitive dysfunction, behavioral changes, and mood disorders. The progressive neurodegeneration of the striatum and other regions like the cerebral cortex leads to the death of patients within 10-20 years after the appearance of the first symptoms [1].

Depending upon the age of disease onset, HD can be classified into two forms: the more traditional adult-onset HD and the less prevalent juvenile-onset HD (JHD), also known as Westphal variant of HD. The mean age of symptom emergence for patients with adult-onset HD is between 30-50 years, while JHD onset occurs before 20 years of age. There is some overlap in symptoms between the two forms; however, the pattern of motor function disruption differs between adult-onset HD and JHD. Choreic movement (abnormal, involuntary movement) is typically the first observed symptom in patients with adult-onset HD.

As the disease progresses, a partial or complete loss of muscle movement, known as hypokinesia, becomes more apparent. In contrast, hypokinesia is often seen from the onset of JHD while chorea is a less prominent symptom in these patients, and in some cases may not be present at all. Epilepsy is often observed in JHD individuals, but epileptic seizures are absent in adult-onset HD. Symptom severity progresses over time, and the average latency from time of HD diagnosis to death is 10-20 years for adult-onset HD patients and less than 10 years for those with JHD.

HD is caused by a genetic defect that results in an expansion of cytosine, adenine, and guanine (CAG) repeats within the huntingtin gene (Htt), leading to the production of mutant huntingtin protein (mHtt). Although the function of wild-type huntingtin protein (Htt) still remains to be fully elucidated, mHtt has been demonstrated to exert toxic effects upon specific neurons within the brain

Htt is expressed ubiquitously throughout the body in multiple subcellular localizations. Although the function of Htt remains to be fully determined, studies have shown that it interacts with an array of other proteins that are involved in several cellular processes including intracellular signaling, metabolism, and gene transcription. Over recent years, increasing evidence has emerged suggesting that the genetic defect in the huntingtin gene results in the disruption of the normal biological functioning of Htt, and that this may play a role in the pathology of HD, in addition to the toxic gain-of-function of mHtt [2-4].

The huntingtin gene is located on chromosome 4p16.3. At the start of this gene in exon 1 lies a stretch of trinucleotide CAG repeats. Each of these triplet repeats codes for the amino acid glutamine, and the repetition of this CAG triplet therefore codes for a string of glutamines, also known as a polyglutamine tract (Huntington's Disease Research Collaborative Group, 1993). The normal huntingtin gene has a polyglutamine tract with a range between six and 26 CAG repeats. The number of these CAG repeats is markedly increased in people who are suffering from HD, and repetitions exceeding 36 are associated with the development of HD [5-6].

The discovery of Huntingtin has yielded new perspectives on the pathogenesis of HD, but the mechanisms leading to the selective death and neuron loss are still unknown.

In parallel with investigations geared to increase understanding about the pathogenesis of HD, efforts are made to find possible therapies for this devastating disease. In this regard, attention has focused on the use of neurotrophic factors in new treatment strategies for human neurodegenerative diseases [7].

BDNF is a member of the neurotrophin family of growth factors which binds specifically to the TrkB tyrosine kinase receptor, thus mediating neurotrophic signalling [8-9]. BDNF is the most abundant neurotrophic factor in the adult brain and it promotes survival, growth and plasticity of various nerve cell populations during normal development and following insults in the adult brain. Given its trophic effects on neurons and its central role in high-order cognitive functions, BDNF has rapidly emerged as a key element in the pathophysiology of numerous brain disorders, including neurological disorders, neurodegenerative diseases and psychiatric disorders.

The fact that BDNF has survival promoting activity on the striatal neurons that die in HD has led to the idea that reduced endogenous trophic support may contribute to disease onset and/or progression. This hypothesis has aroused interest in BDNF and/or BDNF mimetics as potential therapeutic agents, and this has been intensified by reports of reduced BDNF levels in the cerebral cortex and striatum of people with HD as well as in many mouse and cell models of the disease [10-12].

There is a molecular relationship between huntingtin and BDNF as the normal (but not the mutant) huntingtin promotes BDNF production and axonal transport.

Due to Reduction of BDNF Gene's Transcription.

Although no underlying molecular mechanism has been proposed to explain reduced neurotrophic support in other neurological diseases such as Parkinson's disease (PD), or Alzheimer's disease (AD), it is known that the huntingtin mutation in HD reduces the transcriptional activity of the BDNF promoters, thus reducing the transcription of the BDNF gene and decreasing protein production in the cerebral cortex.

This has been confirmed in human by a study, performed on the cerebral cortex, caudate and putamen of patients who had suffered from HD.

This study has also shown that there is a reduction of BDNF expression in the caudate and putamen and suggested that a BDNF surplus may have therapeutic effects in HD.

The wild type huntingtin stimulates BDNF gene transcription by acting at the level of BDNF promoter II, whereas the presence of a pathological CAG expansion in huntingtin abolishes the ability to sustain BDNF transcription in HD.

Due to Reduction of BDNF Transport in HD.

Biochemical studies of mutant huntingtin knock-in cells, mice and HD post-mortem tissues indicated that the complex driving BDNF vesicles is altered in HD. Thus these results could mean that wild-type huntingtin controls the transport of BDNF from cortex to striatum and this transport is affected in HD.

Many studies in mouse and in human tend to attribute the deficit in striatal BDNF in HD to a combination of two factors: a decrease of BDNF production in the cortex and a decrease of the transport of this neurotrophin from the cortex to striatum. Both processes, in which normal huntingtin is involved, are simultaneously disrupted in HD.

Moreover, a report indicates that mutant huntingtin affects TrkB levels in HD by showing that TrkB protein levels are reduced in mutant huntingtin knock-in cells and HD mouse models [13]. A dramatic reduction in TrkB receptors has also been found in striatum from three HD patients and reduced TrkB levels were detected also in cortical samples from four HD subjects. Further investigations are required to understand the extent and consistency of the TrkB downregulation.

In order to overcome problems induced by BDNF reduction in HD, experiments on R6/1 mice have been performed to evaluate the potential in vivo benefits of BDNF supply [14]. It was found that BDNF increased effectively the expression of encephalin as well as the number of encephalin-expressing striatal cells, the most affected cells in HD.

However, despite these promising results, BDNF supplementation raises a number of problems: if the amount is too small it may not be sufficient to produce the required effects, if it is too large, it may be dangerous. Indeed uncontrolled BDNF administration may interfere with other mechanism such as the activity-dependent neuronal plasticity and may induce serious side effects such as epileptic activity [15].

Despite medications available to help manage the symptoms of Huntington's disease, they are currently important unmet needs as no treatments can prevent the physical, mental and behavioral decline associated with the condition.

It is clear that BDNF is one of the critical factors missing in HD, and that an increase of endogenous BDNF production may lead to therapeutics effects, it is very important to control BDNF central and peripheral concentration.

### DETAILED DESCRIPTION OF THE INVENTION

The present invention allows a new therapeutic solution based on Positive Allosteric Modulators (PAM) of TrkB.

It is meant by “positive allosteric modulator” (PAM), also known as allosteric enhancer or potentiator, a compound that induces an amplification of the effect of receptor's response to the primary ligand without directly activating the receptor. Within this invention, PAM TrkB activity is related to the potentation of BDNF effects on the functional activity of the TrkB receptor, measured in vitro or in vivo by mean of a specific TrkB receptor phosphorylation assay.

The compounds and compositions of the invention have several properties such as an effect on neurite outgrowth, a BDNF potentiation, a BBB (Blood Brain Barrier) penetration, a good brain bioavailability, a cell survival increase, a TrkB selectivity and a neuroprotective effect, conferring to this potential PAM an interesting drug's profile which may address some neurodegenerative pathologies, such as Huntington's, Parkinson's and Alzheimer's diseases.

The compounds and compositions of the invention are able to potentiate TrkB-mediated BDNF functional effects and opens a new therapeutic way to threat HD.

In a first aspect, the invention relates to a pharmaceutical composition comprising:

(a) a LIT-TB compound of formula I:

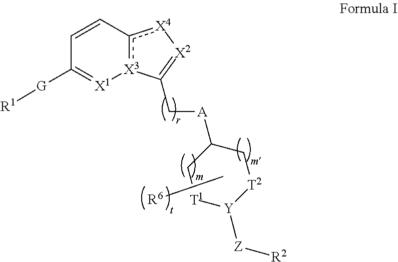

wherein,
R¹ is chosen in the group comprising H, a halogen, a C1 to C10 saturated or unsaturated, substituted or non-substituted, aliphatic, heteroaliphatic, cyclic, alicyclic, heteroalicyclic, aryl, heteroaryl, alkylaryl or alkylheteroaryl group, or R¹ is a group of formula Ia:

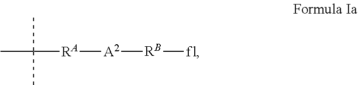

in which,
   R^{A} is a linear C1 to C10 alkyl chain, optionally interrupted by one or more ether or amide functional group,
   A² is an amide functional group,
   R^{B} is an optionally branched C1 to C6 alkyl chain,
   fl is a fluorescent group or a non-fluorescent analogue thereof,
G represents a bond or a -G¹-G²- linker in which
   G¹ is a bond or a C1 to C4 substituted or non-substituted alkyl chain, optionally comprising heteroatoms such as N or O and
   G² represents a C1 to C10 saturated or unsaturated, substituted or non-substituted, aliphatic, heteroaliphatic, cyclic, alicyclic, heteroalicyclic, aryl, heteroaryl, alkylaryl or alkylheteroaryl group,
X¹ and X², identical or different, independently represents CH or N,
X³ is C or N,
X⁴ is N or NH,
Y represents N or CH,
r is an integer from 1 to 3,
A is an amide or amine functional group, preferably A is C(O)NH, NHC(O) or NH,
m is equal to 0, 1 or 2,
m′ is equal to 0, 1 or 2, and m+m′≤3
t is an integer from 0 to 5,
each R⁶ group, identical or different, is chosen in the group comprising H, fluoride, an optionally branched C1 to C6 alkyl chain and a C1 to C6 alkoxy group,
T¹ and T², identical or different, independently represents CH₂, CHR⁶ or C═O,
Z is chosen in the group comprising a bond, H and an optionally branched C1 to C3 alkyl chain, optionally comprising heteroatoms chosen in the group comprising O or N,
R² is null when Z is H or R² is chosen in the group comprising H and a 5- or 6-membered, aromatic or non-aromatic cycle or heterocycle optionally substituted by one or more R⁷ group, each R⁷ group, identical or different, being chosen in the group comprising H, halide, CN, NO₂, NH₂, CONH₂, an optionally branched C1 to C6 alkyl chain and an optionally branched C1 to C6 alkoxy group, two R⁷ groups being optionally covalently bonded to form a cycle,
or a pharmaceutically acceptable salt thereof, and
(b) a pharmaceutically acceptable excipient or carrier.

Within this disclosure, [symbol] represents a single bond or a double bond, depending on the nature of X³ and X⁴, adjacent bonds may be single or double bonds.

Within this disclosure,

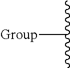

represents a group and its point of attachment to the main molecule.

Pharmaceutically acceptable salts of the compounds of formula I include the acid addition and base salts thereof. Suitable acid addition salts are formed from acids, which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate and xinafoate salts. For a review on suitable salts, see “Handbook of Pharmaceutical Salts: Properties, Selection, and Use” by Stahl and Wermuth (Wiley-VCH, Weinheim, Germany, 2002).

In general, the term “substituted” whether preceded by the term “optionally” or not, and substituents contained in formulae of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds.

As used herein, the term “aliphatic”, refers to non-aromatic groups. Aliphatic groups can be cyclic. Aliphatic groups can be saturated, like hexane, or unsaturated, like hexene and hexyne. Open-chain groups (whether straight or branched) contain no rings of any type, and are thus aliphatic. Aliphatic groups can be saturated, joined by single bonds (alkanes), or unsaturated, with double bonds (alkenes) or triple bonds (alkynes). “Heteroaliphatic” groups are aliphatic groups bearing one or more heteroatom(s), the most common being oxygen, nitrogen and sulfur.

As used herein, the term “alkyl”, refers to straight and branched alkyl groups. An analogous convention applies to other generic terms such as “alkenyl”, “alkynyl” and the like. In certain embodiments, as used herein, “lower alkyl” is used to indicate those alkyl groups (substituted, unsubstituted, branched or unbranched) having about 1-6 carbon atoms. Illustrative alkyl groups include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

In general, the term “aromatic moiety” or “aryl”, as used herein, refers to stable substituted or unsubstituted unsaturated mono- or polycyclic hydrocarbon moieties having preferably 3-14 carbon atoms, comprising at least one ring satisfying the Hackle rule for aromaticity. Examples of aromatic moieties include, but are not limited to, phenyl, indanyl, indenyl, naphthyl, phenanthryl and anthracyl. “Heteroaryl” are both heterocyclic and aromatic.

The term “halogen” as used herein refers to an atom selected from fluorine, chlorine, bromine and iodine.

As used herein, the term “independently” refers to the fact that the substituents, atoms or moieties to which these terms refer, are selected from the list of variables independently from each other (i.e., they may be identical or the same).

As will be understood by the skilled person, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

The skilled person will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

Advantageously, the LIT-TB compound may be chosen in the group of compounds of formula I in which R¹ is chosen in the group comprising H, a C1 to C10 saturated or unsaturated, substituted or non-substituted, aliphatic, heteroaliphatic, cyclic, alicyclic, aryl, heteroaryl, alkylaryl or alkylheteroaryl group. Preferably, R¹ may be chosen in the group comprising H, alkyl group (e.g. methyl, ethyl), cycloalkyl (e.g. cyclopropyl, cyclopentyl), aralkyl (e.g. benzyl, phenethyl), heterocycloaryl (e.g. piperidine), or heteroaryl (e.g. pyridinyl, pyrimidinyl, pyridazinyl, pyrazinyl, 1,2,4-triazinyl, 1,3,5-triazinyl, oxazolyl, imidazolyl), R¹ being optionally substituted.

Advantageously, the LIT-TB compound may be chosen in the group of compounds of formula I in which R¹ is a fluorescent group fl. The fluorescent group fl may be chosen in the group comprising BDP 558/568, BDP 581/591, BDP 630/650, BDP R6G, BDP FL, BDP TMR, BDP TR, coumarin 343, cyanine3, cyanine3.5, cyanine5, cyanine5.5, cyanine7, cyanine7.5, DY-647P1, fluorescein, sulfo-cyanine3, sulfo-cyanine5, sufocyanine5.5, sulfo-cyanine7, sulfo-cyanine7.5, pyrene, rhodamine X, their derivatives, or non-fluorescent analogues thereof.

It is meant by “fluorescent group” (or fluorophore), a group that can re-emit light upon light excitation. Fluorophores typically contain several combined aromatic groups, or planar or cyclic molecules with several π bonds.

As used herein, “derivative” is a compound or group that is derived from a similar compound by a chemical reaction. As an example, florescent group may often be NHS ester prior to attachment. When grafted on the compound, the fluorescent derivative is the same group but without the NHS moiety.

Advantageously, the LIT-TB compound may be chosen in the group of compounds of formula I in which G represents a bond or a -G¹-G²- linker in which G¹ is a bond or a C1 to C4 substituted or non-substituted alkyl chain, optionally comprising heteroatoms such as N or O and G² represents a C1 to C10 saturated or unsaturated, substituted or non-substituted, aliphatic, heteroaliphatic, cyclic, alicyclic, aryl, heteroaryl, alkylaryl or alkylheteroaryl group. Preferably, G¹ may be a bond and G² may be a saturated or unsaturated, substituted or non-substituted C2 to C6 aliphatic or heteroaliphatic group or a saturated or unsaturated, substituted or non-substituted 5-, 6-, or 7-membered cycle or heterocycle.

Advantageously, the LIT-TB compound may be chosen in the group of compounds of formula I in which R¹-G- is linked to the rest of the molecule via a heteroatom, preferably the heteroatom being nitrogen.

Advantageously, the LIT-TB compound may be chosen in the group of compounds of formula I in which R¹-G- is chosen in the group comprising the groups of the following formulae:

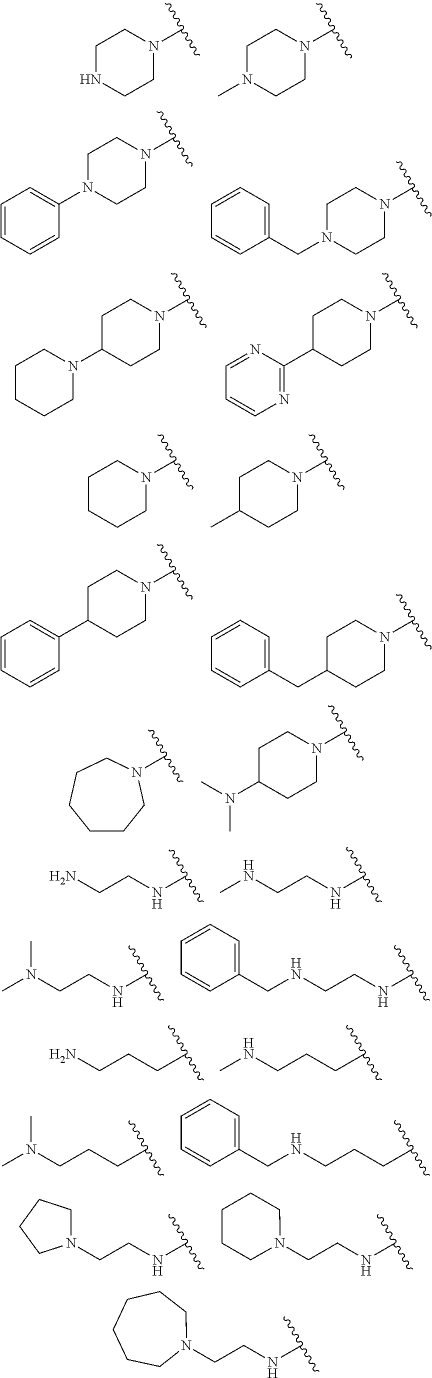

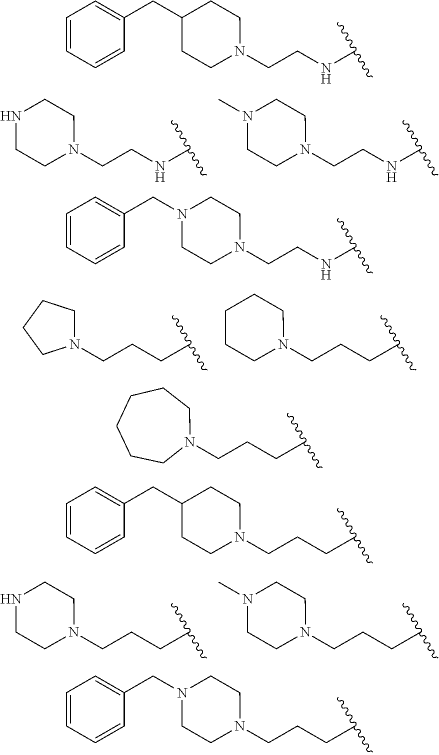

Advantageously, the LIT-TB compound may be chosen in the group of compounds of formula I in which R¹-G- is chosen in the group comprising the groups of the following formulae:

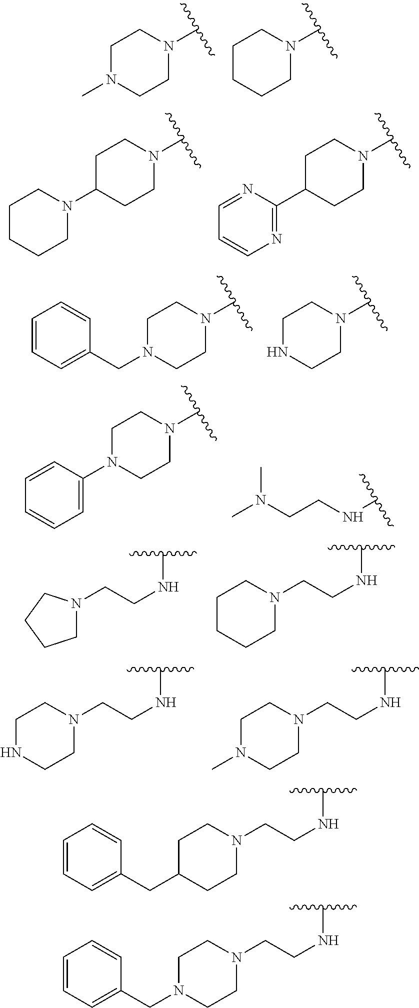

Advantageously, the LIT-TB compound may be chosen in the group of compounds of formula I in which X¹ and X², identical or different, independently may represent CH or N.

Advantageously, the LIT-TB compound may be chosen in the group of compounds of formula I in which X³ may represent C or N.

Advantageously, the LIT-TB compound may be chosen in the group of compounds of formula I in which X⁴ may represent N.

Advantageously, the LIT-TB compound may be chosen in the group of compounds of formula I in which, when X⁴ is N or NH, at least on of X¹, X² and X³ is N. In said group of compounds, X¹, X², X³ may not simultaneously comprise a carbon atom when X⁴ comprises a nitrogen.

Advantageously, X¹ and X² do not represents CH simultaneously.

Advantageously, the LIT-TB compound may be chosen in in the group of compounds of formula I in which X⁴ is N or NH. Preferably, when X⁴ is NH, X³ is C. Preferably, the LIT-TB compound may be chosen in the group of compounds of formula I in which X³ is N and X⁴ is N.

Advantageously, A may be an amide or amine functional group, preferably A is —C(O)NH—, —NHC(O)— or —NH—. Preferably, A is an amide group.

Advantageously, m may be equal to 0, 1 or 2, m′ may be equal to 0, or 2, and m+m′≤3. Preferably, m=m′=1.

Advantageously, t may be an integer from 0 to 5. Preferably, t is 0, or 2.

Advantageously, T¹ and T², identical or different, independently may represent CH₂, CHR⁶ or C═O.

Advantageously, the LIT-TB compound may comprise one or more R⁶ group. The bond going from R⁶ to the center of the cycle indicates that any available position within this cycle may bear an R⁶ group, including T¹ and T². When a carbon atom on the cycle bears an R⁶ group, it replaces an H born by said carbon atom. Each R⁶ group may be identical or different and may be chosen in the group comprising H, fluoride, an optionally branched C1 to C6 alkyl chain and an optionally branched C1 to C6 alkoxy group. Preferably, m=1 and m′=1, t is 0, 1 or 2, R⁶ is F, Cl, Me or OMe, T¹ is CH₂ or C═O and T² is CH₂.

Advantageously, Z may be chosen in the group comprising a bond, H and an optionally branched C1 to C3 alkyl chain, optionally comprising heteroatoms chosen in the group comprising O or N. Preferably, Z is —CH₂—, —CH₂—CH₂— or —CH₂—CH₂—CH₂— or Z is —(CH₂)ₙ—, wherein n is 1, 2 or 3.

Advantageously, the LIT-TB compound may be chosen in the group of compounds of formula I in which R² is chosen in the group comprising H, cycloalkyl (e.g. cyclopentyl), aralkyl (e.g. benzyl, phenethyl) heterocycloaryl (e.g. piperidinyl, piperazyl), or heteroaryl (e.g. pyridinyl, pyrimidinyl, pyridazinyl, pyrazinyl, 1,2,4-triazinyl, 1,3,5-triazinyl, oxazolyl, imidazolyl, furyl, thienyl, pyrrolyl, thiazolyl, pyrrazolyl, 1,3,4-oxadiazolyl, 1,3,4-thiadiazolyl). Optionally, R² is substituted by 1, 2 or 3 R⁷ group(s).

Advantageously, the LIT-TB compound may be chosen in the group of compounds of formula I in which R² is chosen in the group comprising H, cycloalkyl (e.g. cyclopentyl), aralkyl (e.g. benzyl, phenethyl) heterocycloaryl (e.g. piperidine), or heteroaryl (e.g. pyridinyl, pyrimidinyl, pyridazinyl, pyrazinyl, 1,2,4-triazinyl, 1,3,5-triazinyl, oxazolyl, imidazolyl). Optionally, R² is substituted by 1, 2 or 3 R⁷ group(s).

Advantageously, R² may be chosen in group of following formula Ib:

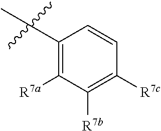

wherein each R^{7a}, R^{7b}, R^{7c} may independently be chosen in the group comprising H, F, Cl, Me, OMe, Et, Pr, iPr, Bu, CN, NO₂, NH₂, CONH₂.

Advantageously, G¹ may be a bond and G² may be —Y¹ (R⁴)—R³—Y²(R⁵)— and the LIT-TB compound may be chosen in the group of compounds of formula II:

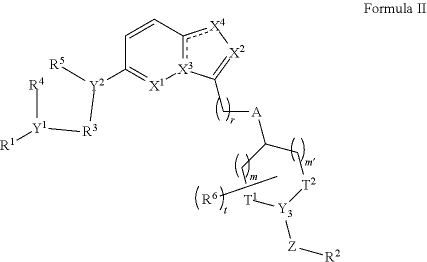

wherein,
R¹, X¹, X², X³, X⁴, r, A, m, m′, t, R⁶, T¹, T², Z and R² are defined as above,
Y¹, Y² and Y³, identical or different, independently represents N of CH,
R⁴ and R⁵, identical or different, are independently chosen in the group comprising H, an optionally branched C1 to C3 alkyl group, optionally comprising heteroatoms chosen in the group comprising O and N, optionally R⁴ and R⁵ may be covalently bonded together to form a cyclic moiety,
R³ is a linear or branched C2 to C6 alkyl chain.

Advantageously, G¹ may be a bond and G² may be —Y¹(R⁴)—R³—Y²(R⁵)— and the LIT-TB compound may be chosen in the group of compounds of formula IIa:

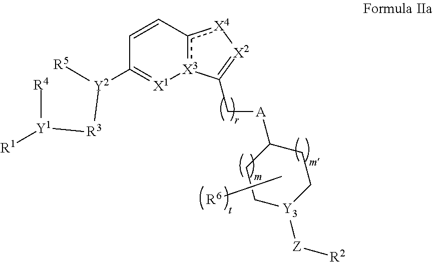

wherein,
R¹, X¹, X², X³, X⁴, r, A, m, m′, t, R⁶, Z, R², Y¹, Y², Y³, R³, R⁴ and R⁵ are defined as above.

Advantageously, G¹ may be a bond and G² may be

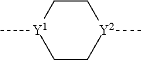

the LIT-TB compound may be chosen in the group of compounds of formula III:

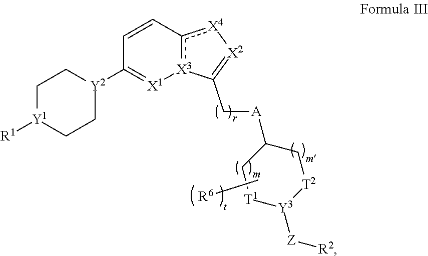

wherein
R¹, X¹, X², X³, X⁴, Y¹, Y², Y³, r, A, m, m′, t, R⁶, T¹, T², Z and R² are defined as above.

Advantageously, G¹ may be a bond and G² may be

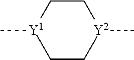

the LIT-TB compound may be chosen in the group of compounds of formula IIIa:

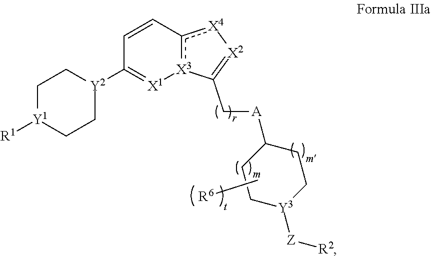

wherein
R¹, X¹, X², X³, X⁴, Y¹, Y², Y³, r, A, m, m′, t, R⁶, Z and R² are defined as above.

Advantageously, X³ and X⁴ are N, Y² is NH, G¹ may be a bond and G² may be Y¹(R⁴)—CH₂—CH₂—NH and the LIT-TB compound may be chosen in the group of compounds of formula IV:

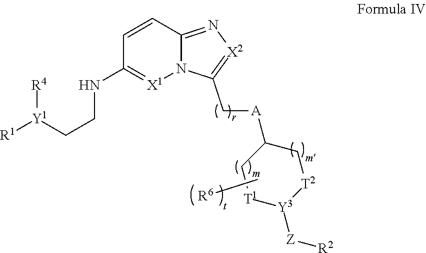

wherein
R¹, R⁴, X¹, X², Y¹, Y³, r, A, m, m′, t, R⁶, T¹, T², Z and R² are defined as above.

Advantageously, X³ and X⁴ are N, Y² is NH, G¹ may be a bond and G² may be Y¹(R⁴)—R³—CH₂—CH₂—NH— and the LIT-TB compound may be chosen in the group of compounds of formula IVa:

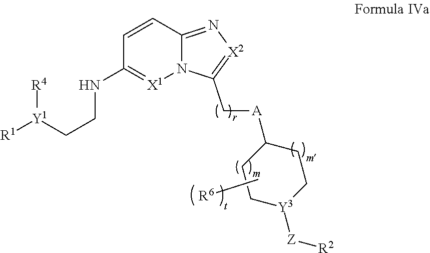

wherein
R¹, R⁴, X¹, X², Y¹, Y³, r, A, m, m′, t, R⁶, Z and R² are defined as above.

Advantageously, the composition may comprise a pharmaceutically acceptable excipient or carrier. In the context of the invention any pharmaceutically acceptable excipient or carrier may be used.

Advantageously, the composition may be an aqueous composition.

Advantageously, the pH of the composition may be comprised in the range 5 to 9.

Advantageously, the concentration of LIT-TB compound of formula I, II, III or IV in the composition may be comprised in the range 1 picoM to 100 μM.

In the present application, when ranges are defined, lower and upper limits are included.

Advantageously, the composition according to the invention may allow a potentialisation of 0.4 nM BDNF response at a concentration of 10 nM of LIT-TB derivative superior or equal to 10%, preferably superior or equal to 20% and more preferably superior or equal to 30%.

Advantageously, the Half maximal effective concentration (EC₅₀) in a TrkB phosphorylation assay is lower or equal to 10 microM.

Advantageously, selectivity is higher or equal to 50 with respect to positive allosteric modulation of related TrkA and TrkC receptors.

On another aspect, the invention relates to a pharmaceutical composition comprising a LIT-TB compound of formula I, II, IIa, III, IIIa, IV or IVa as defined above for use in a drug or in a medicament.

A third aspect of the invention is a pharmaceutical composition comprising a LIT-TB compound of formula I, II, IIa, III, IIIa, IV or IVa as defined above for use in the treatment of neurodegenerative diseases, metabolic disorders, mood disorders, spinal cord injury, brain stroke and ischemia.

In the context of the invention, neurodegenerative diseases may be, but are not limited to, e.g. Alzheimer's disease, amyotrophic lateral sclerosis, Friedreich's disease, Huntington's disease, Lewy body disease, Parkinson's disease, spinal muscular atrophy, metabolic disorders may be, but are not limited to, e.g. obesity, type 2 diabetes mellitus), mood disorders may be, but are not limited to, e.g. depression, anxiety, schizophrenia, bipolar disorders, autism spectrum disorders.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” extend to prophylaxis and include prevent, prevention, preventing, lowering, stopping or reversing the progression or severity of the condition or symptoms being treated. As such, the term “treatment” includes medical, therapeutic, and/or prophylactic administration, as appropriate.

An “effective amount” refers to an amount effective to treat a disease, disorder, and/or condition, or to bring about a recited effect. For example, an amount effective can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of 5 a combination of compounds described herein, e.g., that is effective to treat or prevent a disease or disorder, or to treat the symptoms of the disease or disorder, in a host. Thus, an “effective amount” generally means an amount that provides the desired effect.

On a fourth aspect, the invention relates to compounds of formula I, II, III or IV as defined above, with the exception of N-(1-benzyl-4-piperidyl)-3-[6-(4-methylpiperazin-1-yl)-[1,2,4]triazolo[4,3-b]pyridazin-3-yl]propanamide and N-(1-benzyl-4-piperidyl)-3-[6-(1-piperidyl)-[1,2,4]triazolo[4,3-b]pyridazin-3-yl]propanamide.

Other advantages may also appear to the skilled person when reading the examples below, illustrated by the attached figures, which are given for illustrative purposes only and are not exhaustive.

### BRIEF DESCRIPTION OF THE FIGURES

FIG. 1 represents an effect of LIT-TB001 on Trk phosphorylation, ERK phosphorylation and neurite outgrowth in the presence of NGF/TrkA or BDNF/TrkB. Nnr5 PC12-TrkA and nnr5 PC12-TrkB cells are NGF-nonresponding mutant PC12 cells stably transfected with TrkA and TrkB, respectively [16]. Activation of TrkA and TrkB was assessed in nnr5 PC12-TrkB or -TrkA cells by quantifying the level of phospho-Trk at Tyrosine 706 (Y706) after addition of BDNF (1 nM) or NGF (2 nM), respectively, for 15 min in presence or absence of TB001 at various concentrations (0.1, 10 and nM), as previously done [17]. Activation of downstream signaling pathway was assessed by quantifying phospho-ERK in the same cells. Neurite outgrowth was determined by counting the number the number of cells bearing neurites longer than 2 cells in diameter, 48 hours after initial treatment, as described earlier [17]. In all three assays, LIT-TB001 showed high selectivity toward TrkB signaling, as demonstrated by increased BDNF-, but not NGF-, induced phospho-Trk, phospho-ERK and neurite outgrowth.

FIG. 2 represents the intracellular inhibition of the catalytic activity of 45 kinases (ExpresS Diversity Kinase Panel, Eurofins Discovey, item # P10) by LIT-TB001 at a concentration of 10 μM. For each kinase, the effect of the compound on the ATP-induced kinase-mediated substrate phosphorylation is measured by the TR-FRET LANCE technology.

FIG. 3 represents an effect of acute i.p. administration of LIT-TB001 (0, 0.5 and 1.0 mg/kg) on TrkB phosphorylation in TrkB-expressing regions of the mouse brain. Left: Adult C57BL/6 male mice were injected i.p. with saline (0.9% NaCl) or LIT-TB001 (0.5 or 1 mg/kg). After 1 hour (unless indicated otherwise), mice were decapitated, blood was collected and brains were rapidly removed on ice. Cortex and hippocampus were subsequently dissected and tissues were rapidly processed for western blot analysis using a phosphoY806-TrkB-selective antibody [17]. A representative western blot performed in the cortex of a mouse injected i.p. with saline solution or TB001 (0.5 or 1 mg/kg) for 1 hour is shown. The Anti-TrkB antibody is used to quantify the total amount of TrkB. Anti-tubulin is used as a loading control. Right: phospho-TrkB quantification in the hippocampus and cortex of mice after LIT-TB001 injections shows significant TrkB potentiation in vivo compared to saline treatment (*p<0.05, **p<0.01, one-way ANOVA). Phosphorylated levels of TrkB are calculated as a ratio between phospho and total TrkB bands in each region

### EXAMPLES

### I. Synthetic Methods

The following synthetic methods and schemes illustrate the general procedures by which the compounds of the present invention can be prepared. Starting materials have been obtained from commercially sources or prepared by using methods well known to those of ordinary skill in the art. For example, the compounds of the present invention can be prepared in accordance with or in analogy to the synthetic routes described in detail in the examples section. In particular compounds of the general formula (I) and their pharmaceutically acceptable salts can be synthesized according to methods described in the following schemes where X represents a halogen and R any group at the corresponding position of the general formula (I). While the numbering of the groups R in the following schemes differs from the designation of the groups in the general formula (I), it will be understood that these schemes explain the preparation of compounds of formula (I) and thus these groups R are defined in accordance with the corresponding groups at the same positions in attachment in the general formula (I). Purification of intermediates and final products was carried out via normal or reverse phase chromatography using a Dionex UltiMate 300 with the following parameters: Flow rate of 0.5 mL/min, column temperature: 30° C., solvent system: A (MeOH) and B (0.05% of TFA in H₂O), t=0 min to 1 min: to 60% of B then t=1 min to t=10 min: 60 to 100% of B and t=10 min to t=15 min: 100% of B.

### General Procedure A

Condensation of the N-aralkyl piperidine analogues 1 with cyclic anhydrides 2 afforded the propanoic acid (or homologues) derivatives 4a-c. Starting from ethylmalonyl chloride the condensation led to 2-[(1-benzylpiperidin-4-yl)carbamoyl acetic acid 4c after alkaline hydrolysis of the ester group. Peptide-type coupling of above-mentioned compounds 4 and commercially available 3-chloro-6-hydrazinypyridazine 5 afforded the hydrazide derivatives 6 that were later cyclized under strong acidic conditions at 135° C. into the triazolopyridazines 7. The final compounds of formula 9-14 were last obtained by coupling the 6-chloro-[1,2,4]triazolo[4,3-b]pyridazinederivatives 7 with various heterocyclic secondary amines 8 under basic conditions (Scheme 1).

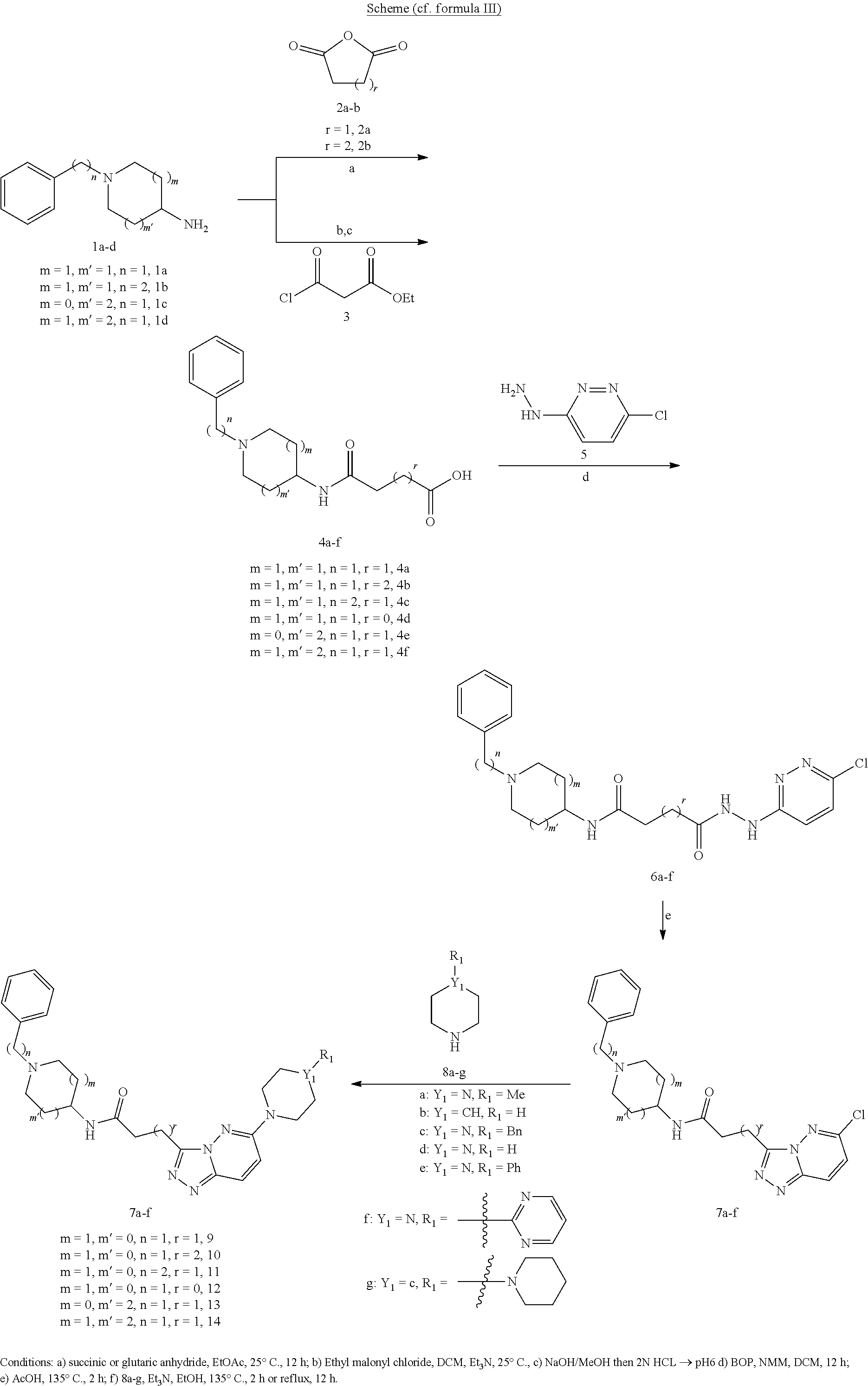

### 4-((1-benzylpiperidin-4-yl)amino)-4-oxobutanoic acid 4a (m′=1, m=1, n=1, r=1)

Succinic anhydride 2a (1.5 eq., 394 mg, 3.94 mmol) was solubilized in EtOAc (5 mL). 4-amino-1-benzylpiperidine 1a (1 eq., 526 mg, 0.566 mL, 2.63 mmol) was added and the reaction mixture was stirred at r.t. overnight (18 h) to yield the carboxylic acid 4a. The white precipitate was filtered and washed with EtOAc (m=763 mg, yield=100%).

¹H NMR (400 MHz, DMSO-d₆) δ 7.76 (d, J=7.7 Hz, 1H), 7.35-7.22 (m, 5H), 3.51 (dtd, J=11.0, 7.0, 3.9 Hz, 1H), 3.45 (s, 2H), 2.77-2.71 (m, 2H), 2.42-2.37 (m, 2H), 2.31-2.26 (m, 2H), 2.00 (ddd, J=11.8, 9.2, 2.5 Hz, 2H), 1.68 (dd, J=12.9, 3.9 Hz, 2H), 1.42-1.31 (m, 2H). ¹³C NMR (101 MHz, DMSO-d₆) δ 173.8, 170.2, 138.4, 128.8, 128.2, 126.9, 62.1, 51.9, 45.9, 31.5, 30.1, 29.2.

### N-(1-benzylpiperidin-4-yl)-4-(2-(6-chloropyridazin-3-yl)hydrazinyl)-4-oxobutanamide (6a) (m′=1, m=1, n=1, r=1)

[(1-Benzylpiperidin-4-yl)carbamoyl]propanoic acid 4a (1 eq., 285 mg, 0.982 mmol), and BOP (1.2 eq., 520 mg, 1.18 mmol) were suspended in DMF (6.3 mL). NMM (1.5 eq., 148 mg, 0.162 mL, 1.47 mmol) was added and the reaction mixture was stirred at r.t. for 15 min. 3-chloro-6-hydrazinylpyridazine 5 (1.2 eq., 170 mg, 1.18 mmol) was then added and the reaction was stirred at r.t. overnight (20 h).

MeOH and silica were added and the crude was evaporated. The adsorbed compound on silica was then purified on silica gel chromatography (eluent MeOH/EtOAc/Et₃N; 1/9/0.3) to yield compound 6a as a yellow solid (m=379 mg, yield=93%).

¹H NMR (500 MHz, Methanol-d₄) δ 7.47 (d, J=9.5 Hz, 1H), 7.39-7.31 (m, 5H), 7.13 (d, J=9.5 Hz, 1H), 3.78-3.70 (m, 3H), 3.03 (d, J=11.4 Hz, 2H), 2.60-2.40 (m, 6H), 1.94-1.87 (m, 2H), 1.64-1.54 (m, 2H). ¹³C NMR (126 MHz, Methanol-d₄) δ 174.7, 173.8, 161.5, 149.6, 131.28, 131.27, 129.66, 129.65, 129.4, 118.4, 63.2, 52.9, 47.0, 31.5, 31.4, 29.9.

### N-(1-benzylpiperidin-4-yl)-3-{6-chloro-[1,2,4]triazolo[4,3-b]pyridazin-3-yl}propanamide 7a (m′=1, m=1, n=1, r=1)

A microwave vial was charged with N-(1-benzylpiperidin-4-yl)-3-[N′-(6-chloropyridazin-3-yl)hydrazine carbonyl]propanamide 6a (1 eq., 361 mg, 0.866 mmol) and acetic acid (2 mL). The vial was properly capped and the mixture vessel was heated at 135° C. for 2 h. The mixture was cooled to r.t. and evaporated. The crude was co-evaporated with cyclohexane and was purified by silica gel chromatography (EtOAc/MeOH/Et₃N, 9/1/0.3) to yield compound 7a as a white solid (m=289 mg, yield=84%).

¹H NMR (400 MHz, Methanol-d₄) δ 8.22 (d, J=9.7 Hz, 1H), 7.40 (d, J=9.7 Hz, 1H), 7.37-7.27 (m, 5H), 3.73-3.62 (m, 3H), 3.43 (t, J=7.4 Hz, 2H), 2.97 (dt, J=12.4, 3.9 Hz, 2H), 2.83 (t, J=7.3 Hz, 2H), 2.36-2.26 (m, 2H), 1.91-1.83 (m, 2H), 1.56 (dtd, J=13.3, 11.2, 3.8 Hz, 2H). ¹³C NMR (101 MHz, Methanol-d₄) δ 173.0, 151.2, 150.9, 144.5, 136.9, 131.0, 129.5, 128.9, 127.2, 124.6, 63.5, 53.0, 47.4, 32.9, 31.7, 21.0.

LC-MS [M+H]⁺=399.17

### Example 1: N-(1-benzylpiperidin-4-yl)-3-[6-(4-methylpiperazin-1-yl)-[1,2,4]triazolo[4,3-b]pyridazin-3-yl] propanamide 9a (LIT-TB001)

N-(1-benzylpiperidin-4-yl)-3-{6-chloro-[1,2,4]triazolo[4,3-b]pyridazin-3-yl}propanamide 7a (1 eq., 191 mg, 0.479 mmol) was solubilized in EtOH (2.5 ml). 1-methylpiperazine 8a (2 eq., 95.9 mg, 0.106 mL, 0.958 mmol) and Et₃N (2 eq., 96.9 mg, 0.133 mL, 0.958 mmol) were added and the reaction was heated at reflux overnight. The product was evaporated and diluted in MeOH. HCl in Et₂O (2M) (excess) was added and the reaction was stirred at r.t. for 1.5 h. The mixture was evaporated and the crude purified by silica gel chromatography using a gradient (AcOEt/MeOH/Et₃N; 9/1/0.5 to 5/1/0.5), salified and lyophilized to yield 9a (LIT-TB001) as a yellowish solid (m=221.2 mg, yield=86%).

¹H NMR (400 MHz, Methanol-d₄) δ 7.87 (d, J=10.2 Hz, 1H), 7.33-7.23 (m, 6H), 3.66-3.59 (m, 5H), 3.49 (s, 2H), 3.35-3.32 (m, 2H), 2.82 (dt, J=12.0, 3.6 Hz, 2H), 2.75 (dd, J=8.0, 7.1 Hz, 2H), 2.58 (t, J=5.1 Hz, 4H), 2.35 (s, 3H), 2.09 (td, J=11.8, 2.6 Hz, 2H), 1.82-1.75 (m, 2H), 1.52-1.41 (m, 2H). ¹³C NMR (101 MHz, Methanol-d₄) δ 173.2, 156.7, 150.0, 143.9, 138.6, 130.7, 129.3, 128.4, 124.7, 116.5, 63.7, 55.4, 53.3, 47.9, 46.4, 46.1, 33.2, 32.3, 21.2.

LC-MS (ESI) [M+H]⁺=463.29

### N-(1-benzylpiperidin-4-yl)-3-[6-(piperidin-1-yl)-[1,2,4]triazolo[4,3-b]pyridazin-3-yl]propanamide 9b (LIT-TB002)

General procedure A for the synthesis of LIT-TB001 analogues was followed using N-(1-benzylpiperidin-4-yl)-3-{6-chloro-[1,2,4]triazolo[4,3-b]pyridazin-3-yl}propanamide 7a (1 eq., 38 mg, 0.0953 mmol), piperidine 8b (2 eq., 16.4 mg, 19 μL, 0.191 mmol) and Et₃N (2 eq., 19.3 mg, 26.5 μL, 0.191 mmol) in EtOH (0.6 ml). The crude was evaporated. A solution of (H₂O/MeOH; 9/1, 1 ml) was added to form a solid. The solid was sonicated, and triturated in presence of heptane, then filtered and washed with heptane to yield the desired product as beige solid. The filtrate was evaporated and purified by reverse phase chromatography (H₂O/MeOH) to give another fraction of the product. Both products were combined, salified and lyophilized to yield 9b (LIT-TB002) as a beige solid (m=24.5 mg, yield=53%).

¹H NMR (400 MHz, Methanol-d₄) δ 7.82 (d, J=10.2 Hz, 1H), 7.33-7.23 (m, 6H), 3.66-3.62 (m, 5H), 3.51 (s, 2H), 3.34-3.31 (m, 2H), 2.84 (d, J=11.6 Hz, 2H), 2.75 (t, J=7.7 Hz, 2H), 2.11 (t, J=11.7 Hz, 2H), 1.80 (d, J=13.1 Hz, 2H), 1.75-1.67 (m, 6H), 1.47 (q, J=11.9 Hz, 2H). ¹³C NMR (101 MHz, Methanol-d₄) δ 173.3, 156.7, 149.9, 143.8, 138.5, 130.7, 129.3, 128.4, 124.3, 116.9, 64.0, 53.3, 48.0, 47.9, 33.2, 32.3, 26.5, 25.5, 21.2.

LC-MS (ESI) [M+H]⁺=448.19

### N-(1-benzylpiperidin-4-yl)-3-[4-benzylpiperazin-1-yl)-[1,2,4]triazolo[4,3-b]pyridazin-3-yl]propan-amide 9c, (LIT-TB005)

General procedure A for the synthesis of LIT-TB001 analogues was followed using N-(1-benzylpiperidin-4-yl)-3-{6-chloro-[1,2,4]triazolo[4,3-b]pyridazin-3-yl}propanamide 7a (1 eq., 100 mg, 0.25 mmol), 1-benzylpiperazine 8c (2 eq., 88.3 mg, 87 μL, 0.5 mmol) and Et₃N (2 eq., 50.7 mg, 70 μL, 0.50 mmol) in EtOH (1.2 ml). Reaction mixture was heated at 135° C. for 2 h. The crude was evaporated and purified by silica gel flash chromatography (EtOAc/MeOH/Et₃N: 9/1/0.5), salified and lyophilized to yield 9c (LIT-TB005) as a brown solid (m=74 mg, yield=55%).

¹H NMR (400 MHz, Methanol-d₄) δ 7.76 (d, J=10.2 Hz, 1H), 7.30-7.12 (m, 11H), 3.57-3.51 (m, 4H), 3.49 (s, 2H), 3.45 (s, 2H), 3.22 (t, J=7.5 Hz, 2H), 2.76 (dt, J=12.4 Hz, J=2.8 Hz, 2H), 2.64 (t, J=7.5 Hz, 2H), 2.55-2.46 (m, 4H), 2.09-2.00 (m, 2H), 1.69 (dt J=12.8 Hz, J=3.8 Hz, 2H), 1.37 (qd, J=11.8 Hz, J=2.8 Hz, 2H). ¹³C NMR (101 MHz, Methanol-d₄) δ 171.8, 168.7, 164.0, 155.4, 148.6, 145.3, 142.5, 137.1, 137.0, 129.3, 129.2, 128.0, 127.9, 127.1, 127.0, 123.2, 115.2, 62.6, 62.4, 52.1, 51.8, 46.5, 45.2, 31.8, 30.919.7 LC-MS (ESI)=538.32 [m/z], 448.27 (-Bn)

### N-(1-benzylpiperidin-4-yl)-3-[6-(piperazin-1-yl)-[1,2,4]triazolo[4,3-b]pyridazin-3-yl]propanamide 9d (LIT-TB007)

General procedure A for the synthesis of LIT-TB001 analogues was followed using N-(1-benzylpiperidin-4-yl)-3-{6-chloro-[1,2,4]triazolo[4,3-b]pyridazin-3-yl}propanamide 7a (1 eq., 110 mg, 0.276 mmol), piperazine 8d (2 eq., 47.5 mg, 0.552 mmol) and Et₃N (2 eq., 55.8 mg, 76.7 μL, 0.552 mmol) in EtOH (2.5 ml). The crude was evaporated and purified by silica gel chromatography (DCM/MeOH/Et₃N; 4/1/0 to 4/1/0.1) to yield 9d (LIT-TB007) as a yellowish solid (m=108 mg, yield=87%).

¹H NMR (400 MHz, Chloroform-d) δ 7.78 (d, J=10.1 Hz, 1H), 7.30-7.18 (m, 4H), 6.89 (d, J=10.1 Hz, 1H), 6.61 (d, J=8.3 Hz, 1H), 3.78-3.70 (m, 1H), 3.52-3.48 (m, 4H), 3.44 (s, 2H), 3.33 (t, J=7.3 Hz, 2H), 2.99-2.95 (m, 4H), 2.84 (t, J=7.2 Hz, 2H), 2.74 (d, J=11.7 Hz, 2H), 2.05 (t, J=11.3 Hz, 2H), 1.80 (dd, J=13.2, 3.8 Hz, 2H), 1.45 (qd, J=11.2, 3.5 Hz, 2H). 13C NMR (101 MHz, Chloroform-d) δ 171.1, 155.1, 148.8, 142.7, 138.4, 129.2, 128.3, 127.1, 124.5, 113.6, 63.1, 52.3, 47.1, 46.7, 45.6, 32.7, 32.0, 20.4.

LC-MS (ES+APCl) [M+H]⁺=449.2

### N-(1-benzylpiperidin-4-yl)-3-[6-(4-phenylpiperazin-1-yl)-[1,2,4]triazolo[4,3-b]pyridazin-3-yl] propanamide 9e (LIT-TB030)

General procedure A for the synthesis of LIT-TB001 analogues was followed using N-(1-benzylpiperidin-4-yl)-3-{6-chloro-[1,2,4]triazolo[4,3-b]pyridazin-3-yl}propanamide 7a (1 eq., 38 mg, 0.0953 mmol), 1-phenylpiperazine 8e (2 eq., 31.9 mg, 30 μL, 0.191 mmol) and Et₃N (2 eq., 19.3 mg, 26.5 μL, 0.191 mmol) in EtOH (0.6 ml). The crude was evaporated. A solution of (H₂O/MeOH; 9/1, 1 ml) was added to form a solid. The solid was sonicated, and triturated in presence of heptane, then filtered and washed with heptane to yield the desired product. The product was salified and lyophilized to yield 9e (LIT-TB030) as a beige solid (m=25.8 mg, yield=49%).

¹H NMR (400 MHz, Methanol-d₄) δ 7.89 (d, J=10.1 Hz, 1H), 7.37 (d, J=10.2 Hz, 1H), 7.33-7.22 (m, 7H), 7.02 (d, J=8.1 Hz, 2H), 6.87 (t, J=7.5 Hz, 1H), 3.79-3.76 (m, 4H), 3.66-3.60 (m, 1H), 3.50 (s, 2H), 3.37-3.28 (m, 6H), 2.83 (d, J=11.8 Hz, 2H), 2.76 (t, J=7.7 Hz, 2H), 2.10 (t, J=11.7 Hz, 2H), 1.78 (d, J=12.8 Hz, 2H), 1.46 (q, J=11.3, 10.6 Hz, 2H), NH (not visible). ¹³C NMR (101 MHz, Methanol-d₄) δ 173.3, 156.8, 152.6, 150.1, 144.0, 138.2, 130.8, 130.2, 129.3, 128.5, 124.7, 121.5, 117.8, 116.7, 63.9, 53.2, 50.4, 47.9, 46.9, 33.3, 32.2, 21.2.

LC-MS (ESI) [M+H]⁺=525.22

### N-(1-benzylpiperidin-4-yl)-3-(6-(4-(pyrimidin-2-yl)piperazin-1-yl)-[1,2,4]triazolo[4,3-b]pyridazin-3-yl)propanamide 9f, (LIT-TB004)

General procedure A for the synthesis of LIT-TB001 analogues was followed using N-(1-benzylpiperidin-4-yl)-3-{6-chloro-[1,2,4]triazolo[4,3-b]pyridazin-3-yl}propanamide 7a (1 eq., 100 mg, 0.25 mmol), 2-(1-piperazinyl)pyrimidine 8f (1 eq., 41.2 mg, 35.5 μL, 0.25 mmol) and Et₃N (2 eq., 50.7 mg, 70 μL, 0.50 mmol) in EtOH (1.2 ml). Reaction mixture was heated at 135° C. for 2 h. The crude was evaporated and purified by silica gel flash chromatography (EtOAc/MeOH/Et3N: 9/1/0.5), salified, triturated with anhydrous Et₂O, and lyophilized to yield 9f (LIT-TB004) as a brown solid (m=50 mg, yield=38%).

LC-MS [M+H]⁺=529.2; 551.2 (M+Na)

### 3-(6-([1,4′-bipiperidin]-1′-yl)-[1,2,4]triazolo[4,3-b]pyridazin-3-yl)-N-(1-benzylpiperidin-4-yl) propanamide 9g, (LIT-TB003)

General procedure A for the synthesis of LIT-TB001 analogues was followed using N-(1-benzylpiperidin-4-yl)-3-{6-chloro-[1,2,4]triazolo[4,3-b]pyridazin-3-yl}propanamide 7a (1 eq., 100 mg, 0.25 mmol), 4-piperidinopiperidine 8g (2 eq., 84.4 mg, 0.50 mmol) and Et₃N (2 eq., 50.7 mg, 70 μL, 0.50 mmol) in EtOH (1.2 ml). Reaction mixture was heated at 135° C. for 2 h. The crude was evaporated and purified by silica gel flash chromatography (EtOAc/MeOH/Et3N: 9/1/0.5), triturated with anhydrous Et₂O, salified and lyophilized to yield 9g (LIT-TB003) as a brown solid (m=100 mg, yield=75%).

LC-MS [M+H]⁺=531.4.

### N-(1-benzylpiperidin-4-yl)-4-(6-(4-methylpiperazin-1-yl)-[1,2,4]triazolo[4,3-b]pyridazin-3-yl)butanamide 10a (LIT-TB009)

General procedure A for the synthesis of LIT-TB001 analogues was followed using N-(1-benzylpiperidin-4-yl)-4-(6-chloro-[1,2,4]triazolo[4,3-b]pyridazin-3-yl)butanamide 7b (1 eq., 100 mg, 0.24 mmol), 1-methylpiperazine 8a (2 eq., 48.5 mg, 0.48 mmol) and Et₃N (2 eq., 49.0 mg, 67 μL, 0.48 mmol) in EtOH (1.1 ml). Reaction mixture was heated at 135° C. for 1.5 h. The crude was evaporated and purified by silica gel flash chromatography (EtOAc/MeOH/Et3N: 9/1/0.5), triturated with anhydrous Et₂O, salified and lyophilised to yield 10a (LIT-TB009) as a brown solid (m=55 mg, yield=48%).

¹H NMR (400 MHz, Methanol-d₄) δ 7.86 (d, 1H, J=10.2 Hz), 7.50-7.42 (m, 2H), 7.37-7.30 (m, 3H), 7.29 (d, 1H, J=10.2 Hz), 3.80-3.60 (m, 5H), 3.40-3.25 (m, 6H), 2.99-3.10 (m, 5H), 2.81 (s, 3H), 2.22 (t, 2H, J=7.2 Hz), 2.10-1.90 (m, 4H), 1.65-1.80 (m 2H). ¹³C NMR (101 MHz, Methanol-d₄) δ 174.7, 156.2, 150.6, 144.0, 132.4, 131.2, 130.6, 130.4, 125.5116.6, 61.4, 54.0, 52.7, 51.9, 44.7, 44.0, 36.0, 29.6, 24.4, 23.3.

LC-MS [M+H]⁺=477.2

### 3-(6-(4-methylpiperazin-1-yl)-[1,2,4]triazolo[4,3-b]pyridazin-3-yl)-N-(1-phenethylpiperidin-4-yl)propanamide 11a (LIT-TB011)

General procedure A for the synthesis of LIT-TB001 analogues was followed using 3-(6-chloro-[1,2,4]triazolo[4,3-b]pyridazin-3-yl)-N-(1-phenethylpiperidin-4-yl)propanamide 7c (1 eq., 100 mg, 0.24 mmol), 1-methylpiperazine 8a (2 eq., 48.5 mg, 0.48 mmol) and Et₃N (2 eq., 49.0 mg, μL, 0.48 mmol) in EtOH (1.1 ml). Reaction mixture was heated at 150° C. under microwaves irradiations 1.5 h. The crude was evaporated and purified by silica gel flash chromatography (EtOAc/MeOH/Et₃N: 9/1/0.5), triturated with anhydrous Et₂O, salified and lyophilized to yield 10a (LIT-TB009) as a light yellow solid (m=70 mg, yield=61%).

¹H NMR (400 MHz, Methanol-d₄) δ 8.2 (d, J=10.2 Hz, 1H), 7.85 (d, J=10.2 Hz, 1H), 7.34-7.07 (m, 5H), 4.58-4.47 (m, 2H), 3.89-3.77 (m, 1H), 3.69-3.57 (m, 4H), 3.48 (t, J=13.3 Hz, 2H), 3.42-3.36 (m, 2H), 3.35-3.22 (m, 4H), 3.06-2.97 (m, 4H), 2.90 (s, 3H), 2.85-2.81 (m, 2H), 2.10-1.88 (m, 2H), 1.82-1.69 (m, 2H)¹³C NMR (101 MHz, Methanol-d₄) 174.6, 156.7, 156.4, 137.5, 137.4, 130.0, 129.8, 128.3, 124.6, 118.5, 59.0, 53.7, 53.0, 45.8, 44.3, 43.6, 32.4, 31.5, 30.2, 20.8

LC-MS [M+H]⁺=477.2

### N-(1-benzylpiperidin-4-yl)-2-(6-(4-methylpiperazin-1-yl)-[1,2,4]triazolo[4,3-b]pyridazin-3-yl)acet-amide 12a (LIT-TB008)

General procedure A for the synthesis of LIT-TB001 analogues was followed using N-(1-benzylpiperidin-4-yl)-2-(6-chloro-[1,2,4]triazolo[4,3-b]pyridazin-3-yl)acetamide 7d (1 eq., 100 mg, 0.26 mmol), 1-methylpiperazine 8a (1.5 eq., 39.0 mg, 0.39 mmol) and Et₃N (2 eq., 52.6 mg, 72 μL, 0.52 mmol) in EtOH (0.75 ml). Reaction mixture was heated at 150° C. under microwaves irradiations for 1.5 h. The crude was evaporated and purified by silica gel flash chromatography (DCM/MeOH/Et₃N: 8/2/0.1), salified and lyophilized to yield 12a (LIT-TB008) as a beige solid (m=70 mg, yield=60%).

¹H NMR (400 MHz, Methanol-d₄) δ 7.96 (d, J=10.2 Hz, 1H), 7.42-7.32 (m, 6H), 4.40-4.29 (m, 2H), 4.20 (s, 2H), 4.05-3.98 (m, 2H), 3.87-3.82 (m, 1H), 3.60-3.52 (m, 2H), 3.43 (d, J=12.4 Hz, 2H), 3.30-3.25 (m, 2H), 3.01 (t, J=12.2 Hz, 2H), 2.86 (s, 3H), 2.12-2.04 (m, 2H), 1.74 (q, J=12.2 Hz, 2H). ¹³C NMR (101 MHz, Methanol-d₄) δ 168.7, 160.1, 156.5, 142.3, 138.6, 132.4, 131.3, 130.4, 125.1117.6, 61.6, 53.7, 52.6, 46.3, 44.3, 43.6, 32.0, 30.0

LC-MS [M+H]⁺=449.2

Alternatively, compounds 9-14 could be prepared in a three-step sequence as illustrated in scheme 2. Condensation of hydrazinopyridazine 5 with cyclic anhydrides 2 in dioxane at 120° C. afforded in one step the triazolo pyridazine propanoic acid (or homologue) 15. Peptide-type coupling of the above mentioned compounds 1 and 15 in presence of isobutyl-chloroformiate led to the previously described triazolo-pyridazine amide 7a-f. Finally, as described in example 1, a nucleophilic aromatic substitution with piperidine or piperazine derivatives 8a-g afforded the products of general formula 9-14.

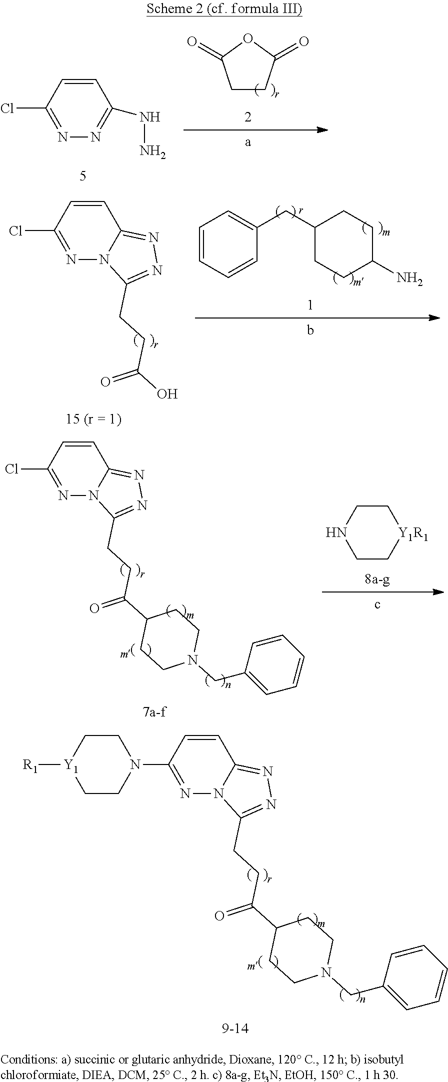

### Example 2: N-(1-benzylpiperidin-3-yl)-3-(6-(4-methylpiperazin-1-yl)-[1,2,4]triazolo[4,3-b]pyridazin-3-yl)propanamide 13a (m=0, m′=2, n=1, r=1) (LIT-TB055)

### Step 1: 3-(6-chloro-[1,2,4]triazolo[4,3-b]pyridazin-3-yl)propanoic acid 15

Succinic anhydride (1.18 eq., 500 mg, 3.46 mmol) was solubilized in dioxane (5 mL). 3-chloro-6-hydrazinylpyridazine 5 (1.18 eq., 420 mg, 0.566 mL, 4.07 mmol) was added and the reaction mixture was heated for 2 hours to yield the triazolo-pyridazinylpropanoic acid 15. The white precipitate was filtered, washed with Et₂O to afford the title compound 15 (m=437 mg, yield=56%).

¹H NMR (400 MHz, DMSO-d₆) δ 11.93 (bs, 1H), 8.44 (d, J=9.6 Hz, 1H), 7.49 (d, J=9.6 Hz, 1H), 3.27 (t, J=7.2 Hz, 2H), 2.88 (t, J=7.2 Hz, 2H). ¹³C NMR (101 MHz, DMSO-d₆) δ 173.5, 149.2, 149.0, 143.3, 127.5, 122.9, 30.2, 19.4

### Step 2: N-(1-benzylpiperidin-3-yl)-3-(6-chloro-[1,2,4]triazolo[4,3-b]pyridazin-3-yl)propanamide 7e

3-(6-chloro-[1,2,4]triazolo[4,3-b]pyridazin-3-yl)propanoic acid 15 (1.0 eq., 119 mg, 0.52 mmol.) was suspended in DCM (3 ml) followed by DIEA (2 eq., 129.2 mg, 0.17 ml, 1.05 mmol). Isobutyl chloroformate (1.2 eq., 86.1 mg, 82.2 μL, 0.63 mmol) in DCM (0.5 mL) was then added dropwise to the solution and the resulting mixture was stirred 30 min at rt. 1-benzylpiperidin-3-amine (1 eq., 100 mg, 0.52 mmol) was then introduced and the agitation was maintained for an additional 2 hours. Volatiles were evaporated and the crude was then purified by silica gel column chromatography using a DCM/MeOH: 90/10 as eluent to yield the title compound 7e as a yellowish solid (m=50 mg, yield=24%).

¹H NMR (400 MHz, Methanol-d₄) δ 8.23 (d, J=9.7 Hz, 1H), 7.42 (d, J=9.7 Hz, 1H), 7.35-7.30 (m, 4H), 7.29-7.24 (m, 1H), 3.94-3.85 (m, 1H), 3.56 (s, 2H), 3.43 (t, J=7.5 Hz, 2H), 2.84 J=7.5 Hz, 2H), 273-2.66 (m, 1H), 2.14 (t, J=11.7 Hz, 1H), 2.04-1.94 (m, 1H), 1.86-1.77 (m, 1H), 1.76-1.68 (m, 1H), 1.66-1.55 (m, 1H), 1.33-1.22 (m, 1H). ¹³C NMR (101 MHz, Methanol-d₄) b 129.2, 127.9, 127.0, 125.8, 123.3, 62.6, 57.5, 52.8, 45.9, 31.5, 29.5, 22.9, 19.6.

### Step 3: N-(1-benzylpiperidin-3-yl)-3-(6-(4-methylpiperazin-1-yl)-[1,2,4]triazolo[4,3-b]pyridazin-3-yl)propanamide (m=0, m′=2, n=1, r=1)

Using the same procedure A described in example 1 for the synthesis of LIT-TB001 analogues and starting from N-(1-benzylpiperidin-3-yl)-3-(6-chloro-[1,2,4]triazolo[4,3-b]pyridazin-3-yl)propanamide 7e (1 eq., 50 mg, 0.12 mmol), 1-methylpiperazine 8a (2 eq., 25.1 mg, 27.8 μL, 0.25 mmol) and Et₃N (2 eq., 25.4 mg, 34.8 μL, 0.25 mmol) in EtOH (0.5 ml), 135° C. for 1.5 h. The title compound 13a was obtained as a yellowish solid after salification and lyophilization (m=28.6 mg, yield=43%).

¹H NMR (400 MHz, Methanol-d₄) δ 7.98 (d, J=10.2 Hz, 1H), 7.51-7.43 (m, 5H), 7.41 (d, J=10.2 Hz, 1H), 4.16 (s, 2H), 4.06-3.96 (m, 1H), 3.90-3.76 (m, 4H), 3.36 (t, J=7.3 Hz, 2H), 3.29-3.15 (m, 2H), 3.10-3.00 (m, 4H), 2.92-2.67 (m, 2H), 2.81 (t, J=12.3 Hz, 2H), 2.68 (s, 3H), 1.99-1.87 (m, 2H), 1.85-1.74 (m, 1H), 1.59-1.47 (m, 1H). ¹³C NMR (101 MHz, Methanol-d₄) δ 173.7, 156.5, 150.0, 144.0, 132.0, 130.6, 130.1, 125.1, 116.6, 62.4, 56.15, 54.6, 53.4, 45.9, 45.5, 44.9, 32.8, 29.0, 22.5, 20.9.

LC-MS [M+H]⁺=462.28

Alternatively compounds 9-14 could also be prepared by reductive amination of the N-BOC-protected pyridazinotriazole 17a-f in presence of appropriate phenylalkyl-aldehydes with the help of sodium cyanoborohydride (Scheme 3). Compounds 17 were readily available from the above described carboxylic acid 15 by peptide type coupling, with commercially available N-BOC protected amino-piperidine derivatives (or homologues) 16, using isobutyl chloroformiate as activated agent (Scheme 3).

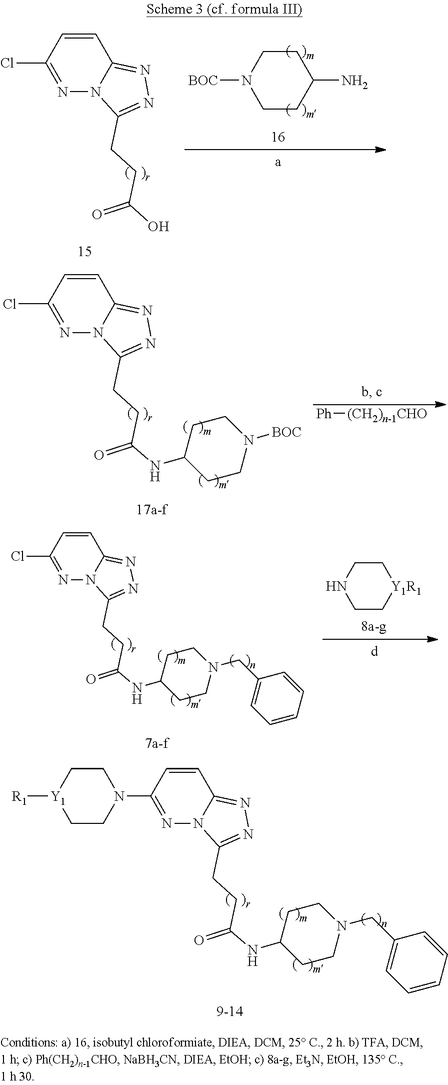

### Example 3: N-(1-benzylazepan-4-yl)-3-(6-(4-methylpiperazin-1-yl)-[1,2,4]triazolo[4,3-b]pyridazin-3-yl)propanamide 14 (m=1, m′=2, n=1, r=1) (LIT-TB056)

### Step 1: tert-butyl 4-(3-(6-chloro-[1,2,4]triazolo[4,3-b]pyridazin-3-yl)propanamido)azepane-1-carboxylate 17f (m=1, m′=2, r=1)

3-(6-chloro-[1,2,4]triazolo[4,3-b]pyridazin-3-yl)propanoic acid 15 (1.0 eq., 116.3 mg, 0.51 mmol) was suspended in DCM (4 ml) followed by DIEA (2 eq., 134.7 mg, 898 μl, 1.04 mmol). Isobutyl chloroformate (1.2 eq., 84.2 mg, 1.20 mL, 0.61 mmol) was dissolved in DCM (0.5 mL, added dropwise to the previous solution and the resulting mixture was stirred 30 min at rt. Tert-Butyl 4-aminoazepane-1-carboxylate (1 eq., 110 mg, 0.51 mmol) was dissolved in DCM (0.5 mL), added dropwise and the agitation was maintained for an additional 2 hours. Volatiles were evaporated and the crude was then purified by silica gel column chromatography using a EtOAc/MeOH: 80/20 as eluent to yield the title compound 17 as a yellowish oil (m=129 mg, yield=59%).

¹H NMR (400 MHz, Methanol-d₄) δ 8.12 (d, J=9.7 Hz, 1H), 7.31 (d, J=9.6 Hz, 1H), 3.69-3.59 (m, 1H), 3.49-3.38 (m, 1H), 3.33 (t, J=7.5 Hz, 2H), 3.32-3.25 (m, 2H), 3.17-3.06 (m, 1H), 2.71 (2.70) (t, J=7.5 Hz, 2H), 1.89-1.79 (m, 1H), 1.78-1.67 (m, 2H), 1.69-1.31 (m, 3H), 1.37 (1.36) (s, 9H, cis-trans geometry). ¹³C NMR (101 MHz, Methanol-d₄) δ 172.4, 157.3, 151.2, 150.9, 144.6, 127.3, 124.7, 81.0 (79.9), 51.2 (51.0), 47.4 (46.8), 44.0 (43.6), 35.7 (35.5), 34.2 (33.9), 32.9, 28.7, 25.6 (25.5), 21.0.

### Step 2: N-(1-benzylazepan-4-yl)-3-{6-chloro-[1,2,4]triazolo[4,3-b]pyridazin-3-yl}propanamide 7f (m=1, m′=2, r=1)

To an ice-cooled solution of tert-butyl 4-(3-(6-chloro-[1,2,4]triazolo[4,3-b]pyridazin-3-yl)propanamido)azepane-1-carboxylate 17f (1 eq., 129 mg, 0.30 mmol,) in DCM (1.5 mL) was added TFA (0.5 mL), and the resulting mixture was stirred for 2 h. The crude reaction was concentrated under vacuum with azeotropic removal of TFA with heptane. The compound was used in the reductive amination step without further purification. The crude was dissolved in MeOH (2 mL). Benzaldehyde (2.2 eq., 71.2 mg, 68 μL) was added followed by NaBH₃CN (3.6 eq. 69 mg, 1.1 mmol). The resulting mixture was stirred at 25° C. overnight. Volatiles were evaporated and the crude was taken up in EtOAc (25 mL). The organic phase was washed with brine, dried and concentrated under vacuum. The residue was purified by silica gel column chromatography using EtOAc: MeOH (90:10) as eluent, to yield 2-(1-benzylpiperidin-4-yl)-4-phenylpyridazin-3(2H)-one as yellow oil (m=92 mg, yield=71%).

¹H NMR (400 MHz, Methanol-d₄) δ 8.17 (d, J=9.6 Hz, 1H), 7.48-7.39 (m, 5H), 7.35 (d, J=9.6 Hz, 1H), 4.21 (s, 2H), 3.93-3.83 (m, 1H), 3.37 (t, J=7.3 Hz, 2H), 3.30-3.08 (m, 4H), 2.77 (t, J=7.3 Hz, 2H), 2.07-1.96 (m, 2H), 1.92-1.73 (m, 3H), 1.63-1.50 (m, 1H). ¹³C NMR (101 MHz, Methanol-d₄) δ 172.9, 151.4, 150.9, 144.7, 132.1, 131.0, 130.4, 127.4, 127.0, 124.8, 62.3, 55.8, 51.5, 50.1, 33.8, 33.0, 30.4, 21.7, 21.0.

### Step 3: N-(1-benzylazepan-4-yl)-3-(6-(4-methylpiperazin-1-yl)-[1,2,4]triazolo[4,3-b]pyridazin-3-yl)propanamide 14

Using the same procedure A described in example 1 for the synthesis of LIT-TB001 analogues and starting from N-(1-benzylazepan-4-yl)-3-{6-chloro-[1,2,4]triazolo[4,3-b]pyridazin-3-yl}propanamide 7f (1 eq., 92 mg, 0.22 mmol), 1-methylpiperazine 8a (2 eq., 40.2 mg, 44.6 μL, 0.40 mmol) and Et₃N (2 eq., 45.2 mg, 62.1 μL, 0.2 mmol) in EtOH (0.5 ml), the title compound 14 was obtained as a yellowish solid after salification and lyophilization (m=23.5 mg, yield=11%).

¹H NMR (400 MHz, Methanol-d₄) δ 7.79 (d, J=10.2 Hz, 1H), 7.36-7.28 (m, 5H), 7.25 (d, J=10.1 Hz, 1H), 3.89 (s, 2H), 3.87-3.79 (m, 1H), 3.56 (t, J=4.5 Hz, 4H), 3.24 (t, J=7.4 Hz, 2H), 2.99-2.75 (m, 4H), 2.66 (t, J=7.4 Hz, 2H), 2.51 (t, J=5.1 Hz, 4H), 2.28 (s, 3H), 1.90-1.80 (m, 2H), 1.79-1.59 (m, 3H), 1.54-1.43 (m, 1H). ¹³C NMR (101 MHz, Methanol-d₄) δ 171.6, 155.3, 152.2, 142.6, 129.9, 128.5, 128.4, 123.3, 115.2, 61.4, 54.7, 53.9, 50.3, 48.7, 45.0, 44.6, 32.6, 31.7, 30.7, 21.7, 19.7.

LC-MS [ESI]: 476.30 (m/z)

General Procedure B

The preparation of compounds of formula 20 bearing diversely substituted piperidines on the propanamide chain can be carried out along various synthetic routes using conventional methods (Scheme 4). Starting from the easily available 6-chloro-triazolopyridazine N-BOC protected piperidine 17a, a SNAr reaction with 8a j led to the corresponding 6-N-methyl piperazine 18a. Deprotection of the protective BOC group and direct alkylation with appropriate halogenoalkylderivatives (Method A, see example 4) or reductive amination with the appropriate aldehyde ((Method B), see example 5) in presence of NaBH(OAc)₃ gave examples of the present invention.

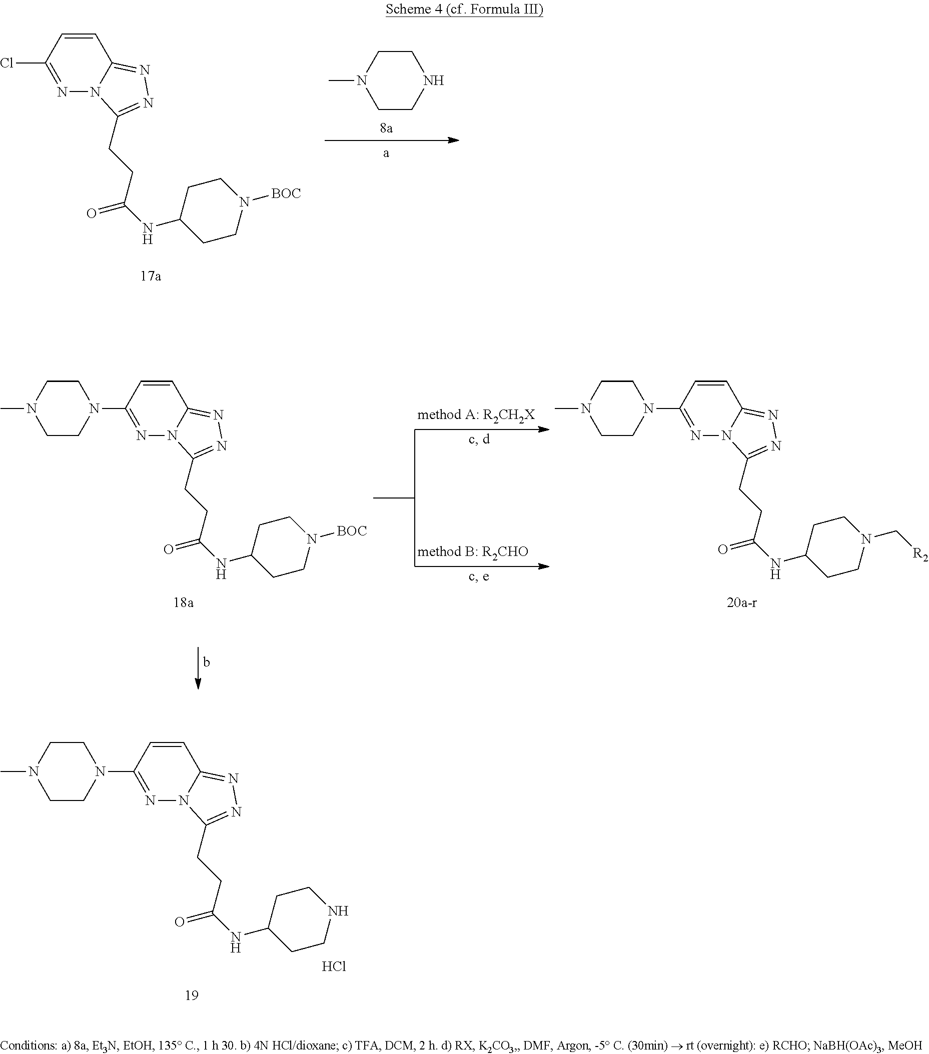

### tert-butyl 4-(3-(6-chloro-[1,2,4]triazolo[4,3-b]pyridazin-3-yl)propanamido)piperidine-1-carboxylate 17a

Using the same procedure described for the preparation of 17f and starting from 3-(6-chloro-[1,2,4]triazolo[4,3-b]pyridazin-3-yl)propanoic acid 15a (1.0 eq., 200 mg, 0.89 mmol) and 4-amino-1-Boc piperidine (1.0 eq., 180 mg, 0.89 mmol, CAS Number: 87120-72-7), the title compound was obtained as a beige solid (m=234 mg, yield=64%).

¹H NMR (400 MHz, DMSO-d₆) δ 8.43 (d, J=9.7 Hz, 1H), 7.95 (d, J=8.0 Hz, 1H), 7.48 (d, J=9.7 Hz, 1H), 3.81 (d, J=14.3 Hz, 2H), 3.75-3.65 (m, 1H), 3.27 (t, J=7.5 Hz, 2H), 2.93-2.75 (m, 2H), 2.68 (t, J=7.5 Hz, 2H), 1.68 (dd, J=12.9 Hz, J=4.1 Hz, 2H), 1.39 (s, 9H), 126-1.14 (m, 2H). ¹³C NMR (100 MHz, DMSO-d₆) δ 170.1, 154.4, 149.4, 149.1, 143.2, 127.4, 122.8, 79.1, 46.1, 32.0, 31.8, 28.5, 20.0.

### tert-butyl 4-(3-(6-(4-methylpiperazin-1-yl)-[1,2,4]triazolo[4,3-b]pyridazin-3-yl)propanamido) piperidine-1-carboxylate 18a

Using the General procedure A for the synthesis of LIT-TB001 analogues was followed using tert-butyl 4-(3-(6-chloro-[1,2,4]triazolo[4,3-b]pyridazin-3-yl)propanamido)piperidine-1-carboxylate 17a (1 eq., 50 mg, 0.12 mmol), 1-methyl-piperazine 8a (2 eq., 16.4 mg, 19 μL, 0.191 mmol) and Et₃N (2 eq., 24.75 mg, 34 μL, 0.24 mmol) in EtOH (0.8 ml). The crude was evaporated, purified by reverse phase chromatography (H₂O/MeOH) to give the title compound as a white solid (m=45 mg, yield=78%).

¹H NMR (400 MHz, Methanol-d₄) δ 7.90 (d, J=10.2 Hz, 1H), 7.36 (d, J=10.2 Hz, 1H), 3.98 (d, J=13.7 Hz, 2H), 3.81 (tt, J=10.8, 4.1 Hz, 1H), 3.68 (t, J=5.2 Hz, 4H), 3.36 (dd, J=7.9, 7.2 Hz, 2H), 2.96-2.85 (m, 2H), 2.78 (t, J=7.5 Hz, 2H), 2.62 (t, J=5.1 Hz, 4H), 2.38 (s, 3H), 1.80 (dd, J=13.1, 3.8 Hz, 2H), 1.46 (s, 9H), 1.37-1.25 (m, 2H). ¹³C NMR (101 MHz, Methanol-d₄) δ 173.2, 156.8, 156.4, 150.0, 144.0, 124.7, 116.6, 81.1, 55.4, 47.9, 46.46, 46.44, 46.1, 33.2, 32.6, 28.7, 21.1.

### 3-(6-(4-methylpiperazin-1-yl)-[1,2,4]triazolo[4,3-b]pyridazin-3-yl)-N-(piperidin-4-yl)propanamide 19 (LIT-TB021)

Tert-butyl 4-{3-[6-(4-methylpiperazin-1-yl)-[1,2,4]triazolo[4,3-b]pyridazin-3-yl]propanamido} piperidi-ne-1-carboxylate 18a (67 mg, 0.14 mmol) was solubilized in DCM (0.7 mL). A solution of 4N HCl in dioxane was added (10 eq., 1.42 mmol, 0.35 ml) was added and the reaction mixture was stirred at r.t. for 30 min. Precipitate was collected, washed thrice with dry Et₂O, and dried (m=27 mg, yield=43%)

¹H NMR (400 MHz, Methanol-d₄) δ 7.89 (d, J=10.2 Hz, 1H), 7.34 (d, J=10.2 Hz, 1H), 3.79-3.68 (m, 1H), 3.66 (t, J=5.1 Hz, 4H), 3.34 (t, J=7.6 Hz, 2H), 3.03 (dt, J=12.7 Hz, J=4.1 Hz, 2H), 2.76 (t, J=7.6 Hz, 2H), 2.65 (td, J=12.2 Hz, J=2.8 Hz, 2H), 2.60 (t, J=5.1 Hz, 4H), 2.36 (s, 3H), 1.81 (dd, J=12.9 Hz, J=3.8 Hz, 2H), 1.37 (qd, J=12.0 Hz, J=6.0 Hz). 13C NMR (101 MHz, Methanol-d₄) δ 173.3, 156.9, 150.2, 144.1, 124.9, 116.8, 55.5, 48.0, 46.6, 46.3, 45.8, 33.3, 33.1, 21.3.

LC-MS [M+H]⁺=372.24

### Example 4: N-{1-[(4-methoxyphenyl)methyl]piperidin-4-yl}-3-[6-(4-methylpiperazin-1-yl)-[1,2,4] triazolo [4,3-b]pyridazin-3-yl]propanamide 20a (LIT-TB017)

Tert-butyl 4-{3-[6-(4-methylpiperazin-1-yl)-[1,2,4]triazolo[4,3-b]pyridazin-3-yl]propanamido} piperidi-ne-1-carboxylate 18a (17.2 mg, 0.0364 mmol) was solubilized in DCM (0.3 mL). TFA (10 eq., 41.5 mg, 27 μL, 0.364 mmol) was added and the reaction mixture was stirred at r.t. for 2 h. The crude was evaporated, then co-evaporated twice with DCM/heptane. After drying, the crude was solubilized in dry DMF under Argon. K₂CO₃ (5 eq., 25.2 mg, 0.182 mmol) was added and the reaction mixture was stirred at −5° C. for 30 min. The 1-(bromomethyl)-4-methoxybenzene (1 eq., 7.32 mg, 5.25 μL, 0.0364 mmol).) was added, and the mixture was stirred at −5° C. for 0.5 h then at r.t. overnight. Water (few drops) was added and the crude was directly purified by reverse phase chromatography (H₂O/MeOH). The product was evaporated and diluted in MeOH. HCl in Et₂O (2M) (excess) was added and the reaction was stirred at r.t. for 1.5 h. The mixture was evaporated, diluted in water and lyophilized. The title compound 20a was obtained as a yellowish solid (m=6.9 mg, yield=30%).

¹H NMR (400 MHz, Methanol-d₄) δ 7.89 (d, J=10.2 Hz, 1H), 7.35 (d, J=10.2 Hz, 1H), 7.24 (d, J=8.1 Hz, 2H), 6.89 (d, J=8.1 Hz, 2H), 3.79 (s, 3H), 3.68-3.60 (m, 5H), 3.52 (s, 2H), 3.36-3.31 (m, 2H), 2.88 (d, J=11.6 Hz, 2H), 2.76 (t, J=7.7 Hz, 2H), 2.61-2.58 (m, 4H), 2.37 (s, 3H), 2.18 (t, J=11.6 Hz, 2H), 1.81 (d, J=13.0 Hz, 2H), 1.48 (q, J=12.0 Hz, 2H), NH (not visible). 13C NMR (126 MHz, Methanol-d₄) δ 173.3, 160.8, 156.8, 150.1, 143.9, 132.2, 129.5, 124.7, 116.6, 114.8, 63.1, 55.7, 55.4, 53.0, 47.7, 46.4, 46.1, 33.2, 32.0, 21.2.

LC-MS (ESI) [M+H]⁺=493.20

### N-{1-[(3-chlorophenyl)methyl]piperidin-4-yl}-3-[6-(4-methylpiperazin-1-yl)-[1,2,4]triazolo[4,3-b]pyridazin-3-yl]propanamide 20b (LIT-TB018)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 14.9 mg, 0.0315 mmol), 3-chlorobenzyl bromide (1.1 eq., 7.35 mg, 4.69 μL, 0.0347 mmol). and K₂CO₃ (5 eq., 21.8 mg, 0.158 mmol) in DMF (0.3 ml). The crude was evaporated and purified by reverse phase chromatography (H₂O/MeOH), salified and lyophilized to yield 20b as a yellowish solid (m=9.4 mg, yield=52%).

¹H NMR (400 MHz, Methanol-d₄) δ 7.89 (d, J=10.1 Hz, 1H), 7.39-7.22 (m, 5H), 3.67-3.50 (m, 5H), 3.50 (s, 2H), 3.33 (t, J=11.0 Hz, 2H), 2.82 (d, J=11.8 Hz, 2H), 2.76 (t, J=7.6 Hz, 2H), 2.61-2.58 (m, 4H), 2.36 (s, 3H), 2.12 (t, J=11.8 Hz, 2H), 1.80 (d, J=12.9 Hz, 2H), 1.47 (q, J=11.9 Hz, 2H). ¹³C NMR (126 MHz, Methanol-d₄) δ 173.2, 156.8, 150.1, 143.9, 141.3, 135.3, 130.8, 130.4, 128.9, 128.5, 124.7, 116.6, 63.2, 55.4, 53.3, 47.9, 46.4, 46.1, 33.2, 32.4, 21.2.

LC-MS (ESI) [M+H]⁺=497.17

### N-{1-[(2-chlorophenyl)methyl]piperidin-4-yl}-3-[6-(4-methylpiperazin-1-yl)-[1,2,4]triazolo[4,3-b]pyridazin-3-yl]propanamide 20c (LIT-TB019)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 14.8 mg, 0.0313 mmol), 2-chlorobenzyl bromide (1.1 eq., 7.08 mg, 4.47 μL, 0.0344 mmol) and K₂CO₃ (5 eq., 21.6 mg, 0.157 mmol) in DMF (0.3 ml). The crude was evaporated and purified by reverse phase chromatography (H₂O/MeOH), salified and lyophilized to yield 20c as a yellowish solid (m=11.2 mg, yield=63%).

¹H NMR (400 MHz, Methanol-d₄) δ 7.88 (d, J=10.0 Hz, 1H), 7.47 (d, J=6.9 Hz, 1H), 7.42 (d, J=6.8 Hz, 1H), 7.38-7.27 (m, 3H), 3.83 (s, 2H), 3.72-3.69 (m, 5H), 3.35-3.31 (m, 2H), 3.02 (d, J=11.8 Hz, 2H), 2.80-2.69 (m, 6H), 2.49-2.43 (m, 2H), 2.45 (s, 3H), 1.86 (d, J=12.9 Hz, 2H), 1.57 (q, J=11.5 Hz, 2H. ¹³C NMR (101 MHz, Methanol-d₄) δ 173.2, 156.8, 150.1, 143.9, 132.4, 130.5, 129.7, 127.9, 124.7, 116.5, 60.1, 55.4, 53.4, 47.9, 46.4, 46.1, 33.2, 32.5, 21.2.

LC-MS (ESI) [M+H]1=497.16

### N-{1-[(4-fluorophenyl)methyl]piperidin-4-yl}-3-[6-(4-methylpiperazin-1-yl)-[1,2,4]triazolo[4,3-b]pyridazin-3-yl]propanamide 20d (LIT-TB020)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 16.3 mg, 0.0345 mmol), 4-fluorobenzyl chloride (1.1 eq., 5.49 mg, 4.52 μL, 0.0379 mmol) and K₂CO₃ (5 eq., 23.8 mg, 0.172 mmol) in DMF (0.3 ml). The crude was evaporated and purified by reverse phase chromatography (H₂O/MeOH), salified and lyophilized to yield 20d as a yellowish solid (m=5.1 mg, yield=27%).

¹H NMR (400 MHz, Methanol-d₄) δ 7.88 (d, J=10.2 Hz, 1H), 7.35-7.31 (m, 3H), 7.04 (t, J=8.6 Hz, 2H), 3.67-3.62 (m, 5H), 3.50 (s, 2H), 3.35-3.30 (m, 2H), 2.83 (d, J=11.5 Hz, 2H), 2.75 (t, J=7.6 Hz, 2H), 2.60-2.58 (m, 4H), 2.35 (s, 3H), 2.12 (t, J=11.8 Hz, 2H), 1.79 (d, J=12.9 Hz, 2H), 1.46 (q, J=12.0 Hz, 2H). ¹³C NMR (126 MHz, Methanol-d₄) δ 173.2, 163.6 (d, J=244.1 Hz), 156.8, 150.1, 143.9, 134.6 (d, J=3.2 Hz), 132.5 (d, J=8.0 Hz), 124.7, 116.6, 115.9 (d, J=21.5 Hz), 63.0, 55.4, 53.2, 47.9, 46.4, 46.1, 33.2, 32.4, 21.2. ¹⁹F NMR (376 MHz, Methanol-d₄) δ−117.5.

LC-MS (ESI) [M+H]⁺=481.18

### N-{1-[(2-fluorophenyl)methyl]piperidin-4-yl}-3-[6-(4-methylpiperazin-1-yl)-[1,2,4]triazolo[4,3-b]pyridazin-3-yl]propanamide 20e (LIT-TB022)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 16.3 mg, 0.0345 mmol), 2-fluorobenzyl bromide (1.1 eq., 7.17 mg, 4.58 μL, 0.0379 mmol) and K₂CO₃ ((5 eq., 23.8 mg, 0.172 mmol) in DMF (0.3 ml). The crude was evaporated and purified by reverse phase chromatography (H₂O/MeOH), salified and lyophilized to yield 20e as a yellowish solid (10.9 mg, yield=57%).

¹H NMR (400 MHz, Methanol-d₄) δ 7.88 (d, J=10.1 Hz, 1H), 7.42-7.27 (m, 3H), 7.15 (t, J=7.6 Hz, 1H), 7.08 (t, J=9.4 Hz, 1H), 3.67-3.65 (m, 5H), 3.59 (s, 2H), 3.35-3.31 (m, 2H), 2.86 (d, J=11.8 Hz, 2H), 2.75 (t, J=7.7 Hz, 2H), 2.60-2.58 (m, 4H), 2.36 (s, 3H), 2.17 (t, J=11.7 Hz, 2H), 1.79 (d, J=12.9 Hz, 2H), 1.47 (q, J=12.0 Hz, 2H). ¹³C NMR (126 MHz, Methanol-d₄) δ 173.2, 162.9 (d, J=245.2 Hz), 156.8, 150.1, 143.9, 133.3 (d, J=4.2 Hz), 130.6 (d, J=8.3 Hz), 125.1 (d, J=3.6 Hz), 125.0, 124.7, 116.6, 116.2 (d, J=22.6 Hz), 56.0 (d, J=1.9 Hz), 55.4, 53.1, 47.8, 46.4, 46.1, 33.2, 32.3, 21.2. ¹⁹F NMR (376 MHz, Methanol-d₄) δ−119.35.

LC-MS (ESI) [M+H]⁺=481.19

### 3-[6-(4-methylpiperazin-1-yl)-[1,2,4]triazolo[4,3-b]pyridazin-3-yl]-N-[1-(1-phenylethyl)piperidin-4-yl]propanamide 20f (LIT-TB023)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 19.4 mg, 0.0411 mmol), (1-bromoethyl)benzene (1.1 eq., 8.36 mg, 6.19 μL, 0.0452 mmol) and K₂CO₃ (5 eq., 28.4 mg, 0.205 mmol) in DMF (0.3 ml). The crude was evaporated and purified by reverse phase chromatography (H₂O/MeOH), salified and lyophilised to yield 20f as a yellowish solid (m=14.4 mg, yield=64%). ¹H NMR (400 MHz, Methanol-d₄) δ 7.88 (d, J=10.0 Hz, 1H), 7.36-7.22 (m, 6H), 3.66-3.64 (m, 4H), 3.57 (t, J=11.4 Hz, 1H), 3.48 (q, J=6.7 Hz, 1H), 3.35-3.31 (m, 2H), 3.07 (d, J=11.6 Hz, 1H), 2.80-2.72 (m, 3H), 2.60-2.57 (m, 4H), 2.35 (s, 3H), 2.08 (dt, J=34.5, 11.9 Hz, 2H), 1.78 (dd, J=34.3, 13.0 Hz, 2H), 1.56-1.38 (m, 2H), 1.41 (d, J=6.7 Hz, 3H). ¹³C NMR (126 MHz, Methanol-d₄) δ 173.2, 156.8, 150.1, 143.9, 143.4, 129.4, 129.0, 128.5, 124.7, 116.6, 66.4, 55.4, 51.0, 50.4, 47.9, 46.4, 46.1, 33.2, 32.4, 21.2, 19.7.

LC-MS (ESI) [M+H]⁺=477.21

### N-{1-[(2-methylphenyl)methyl]piperidin-4-yl}-3-[6-(4-methylpiperazin-1-yl)-[1,2,4]triazolo[4,3-b]pyridazin-3-yl]propanamide 20g (LIT-TB024)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 17.7 mg, 0.0375 mmol), 2-methylbenzyl bromide (1.1 eq., 7.62 mg, 5.52 μL, 0.0412 mmol) and K₂CO₃ (5 eq., 25.9 mg, 0.187 mmol) in DMF (0.3 ml). The crude was evaporated and purified by reverse phase chromatography (H₂O/MeOH), salified and lyophilized to yield 20g as a yellowish solid (m=13.3 mg, yield=65%).

¹H NMR (400 MHz, Methanol-d₄) δ 7.88 (d, J=10.2 Hz, 1H), 7.33 (d, J=10.1 Hz, 1H), 7.23-7.21 (m, 1H), 7.14-7.10 (m, 3H), 3.67-3.64 (m, 5H), 3.49 (s, 2H), 3.35-3.31 (m, 2H), 2.85 (d, J=11.5 Hz, 2H), 2.75 (t, J=7.6 Hz, 2H), 2.61-2.58 (m, 4H), 2.36 (s, 3H), 2.35 (s, 3H), 2.15 (t, J=11.8 Hz, 2H), 1.78 (d, J=12.8 Hz, 2H), 1.45 (q, J=11.9 Hz, 2H). ¹³C NMR (126 MHz, Methanol-d₄) δ 173.2, 156.8, 150.1, 143.9, 138.7, 137.0, 131.4, 131.2, 128.4, 126.6, 124.7, 116.6, 61.4, 55.4, 53.5, 48.0, 46.4, 46.1, 33.2, 32.5, 21.2, 19.5.

LC-MS (ESI) [M+H]⁺=477.24

### 3-[6-(4-methylpiperazin-1-yl)-[1,2,4]triazolo[4,3-b]pyridazin-3-yl]-N-[1-(pyridin-4-ylmethyl)piperidin-4-yl]propanamide 20h (LIT-TB025)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 20.5 mg, 0.0434 mmol), 4-(chloromethyl)pyridine hydrochloride (1.1 eq., 7.83 mg, 0.0477 mmol) and K₂CO₃ (5 eq., 30 mg, 0.217 mmol) in DMF (0.3 ml). The crude was evaporated and purified by reverse phase chromatography (H₂O/MeOH), salified and lyophilized to yield 20h as a yellowish solid (m=13.9 mg, yield=56%).

¹H NMR (500 MHz, Methanol-d₄) δ 8.49-8.43 (m, 2H), 7.88 (d, J=10.2 Hz, 1H), 7.43-7.40 (m, 2H), 7.33 (d, J=10.2 Hz, 1H), 3.68-3.60 (m, 5H), 3.56 (s, 2H), 3.35-3.31 (m, 2H), 2.80 (d, J=11.9 Hz, 2H), 2.75 (t, J=7.6 Hz, 2H), 2.61-3.58 (m, 4H), 2.36 (s, 3H), 2.14 (td, J=11.8, 2.5 Hz, 2H), 1.83-1.76 (m, 2H), 1.53-1.44 (m, 2H). ¹³C NMR (126 MHz, Methanol-d₄) δ 173.2, 156.8, 150.3, 150.1, 150.0, 144.0, 125.8, 124.7, 116.6, 62.5, 55.4, 53.5, 47.9, 46.4, 46.1, 33.2, 32.5, 21.2.

LC-MS (ESI) [M+H]⁺=464.18

### N-{1-[(3,4-dichlorophenyl)methyl]piperidin-4-yl}-3-[6-(4-methylpiperazin-1-yl)-[1,2,4]triazolo[4,3-b]pyridazin-3-yl]propanamide 20i (LIT-TB026)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 19.2 mg, 0.0406 mmol), 3,4-dichlorobenzyl chloride (1.1 eq., 8.74 mg, 6.2 μL, 0.0447 mmol) and K₂CO₃ (5 eq., 28.1 mg, 0.203 mmol) in DMF (0.3 ml). The crude was evaporated and purified by reverse phase chromatography (H₂O/MeOH), salified and lyophilized to yield 20i as a yellowish solid (m=15.6 mg, yield=64%).

¹H NMR (500 MHz, Methanol-d₄) δ 7.88 (d, J=10.1 Hz, 1H), 7.50 (d, J=2.0 Hz, 1H), 7.45 (d, J=8.2 Hz, 1H), 7.33 (d, J=10.2 Hz, 1H), 7.24 (dd, J=8.2, 2.0 Hz, 1H), 3.69-3.58 (m, 5H), 3.47 (s, 2H), 3.35-3.31 (m, 2H), 2.83-2.77 (m, 2H), 2.75 (t, J=7.6 Hz, 2H), 2.60-2.57 (m, 4H), 2.35 (s, 3H), 2.11 (td, J=11.8, 2.6 Hz, 2H), 1.79 (dd, J=12.9, 3.9 Hz, 2H), 1.53-1.41 (m, 2H). ¹³C NMR (126 MHz, Methanol-d₄) δ 173.2, 156.8, 150.1, 143.9, 140.1, 133.2, 132.3, 132.0, 131.4, 130.2, 124.7, 116.6, 62.5, 55.4, 53.3, 47.9, 46.4, 46.1, 33.2, 32.4, 21.2.

LC-MS (ESI) [M+H]⁺=531.11

### N-(1-benzoylpiperidin-4-yl)-3-[6-(4-methylpiperazin-1-yl)-[1,2,4]triazolo[4,3-b]pyridazin-3-yl]propanamide hydrochloride 20 j (LIT-TB027)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 20.2 mg, 0.0427 mmol), 3 benzoyl chloride (1.1 eq., 6.61 mg, 5.46 μL, 0.047 mmol) and K₂CO₃ (5 eq., 29.5 mg, 0.214 mmol) in DMF (0.3 ml). The crude was evaporated and purified by reverse phase chromatography (H₂O/MeOH), salified and lyophilized to yield 20j as a yellowish solid (m=8.2 mg, yield=32%).

¹H NMR (400 MHz, Methanol-d₄) δ 7.91 (d, J=10.2 Hz, 1H), 7.48-7.44 (m, 3H), 7.41-7.35 (m, 3H), 4.47 (d, J=13.4 Hz 1H), 3.93 (tt, J=10.5, 4.2 Hz, 1H), 3.77-3.63 (d, J=5.3 Hz, 5H), 3.36 (t, J=7.4 Hz, 2H), 3.23-3.02 (m, 2H), 2.83-2.76 (m, 6H), 2.50 (s, 3H), 2.00-1.73 (m, 2H), 1.51-1.30 (m, 2H. ¹³C NMR (126 MHz, Methanol-d₄) δ 173.3, 172.5, 156.8, 150.0, 144.0, 137.0, 131.1, 129.8, 127.8, 124.7, 116.6, 55.4, 47.8, 46.4, 46.1, 42.1, 33.2, 32.2, 21.1.

LC-MS (ESI) [M+H]⁺=477.17

### N-{1-[(4-chlorophenyl)methyl]piperidin-4-yl}-3-[6-(4-methylpiperazin-1-yl)-[1,2,4]triazolo[4,3-b]pyridazin-3-yl]propanamide 20k (LIT-TB028)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 19.8 mg, 0.0419 mmol), 4-chlorobenzyl bromide (1.1 eq., 9.47 mg, 0.0461 mmol) (1.1 eq., 6.61 mg, 5.46 μL, 0.047 mmol) and K₂CO₃ (5 eq., 29 mg, 0.209 mmol) in DMF (0.3 ml). The crude was evaporated and purified by reverse phase chromatography (H₂O/MeOH), salified and lyophilized to yield 20k as a yellowish solid (m=10.8 mg, yield=45%).

¹H NMR (500 MHz, Methanol-d₄) δ 7.88 (d, J=10.1 Hz, 1H), 7.33 (d, J=10.2 Hz, 1H), 7.31-7.29 (m, 4H), 3.69-3.58 (m, 5H), 3.48 (s, 2H), 3.35-3.31 (m, 2H), 2.81 (d, J=11.8 Hz, 2H), 2.74 (t, J=7.5 Hz, 2H), 2.60-2.57 (m, 4H), 2.35 (s, 3H), 2.10 (td, J=11.8, 2.5 Hz, 2H), 1.78 (dd, J=13.5, 3.7 Hz, 2H), 1.54-1.41 (m, 2H). ¹³C NMR (126 MHz, Methanol-d₄) δ 173.2, 156.8, 150.1, 143.9, 137.5, 134.2, 132.2, 129.4, 124.7, 116.6, 63.0, 55.4, 53.2, 47.9, 46.4, 46.1, 33.2, 32.4, 21.2.

LC-MS (ESI) [M+H]+=497.16

### N-[1-(cyclohexylmethyl)piperidin-4-yl]-3-[6-(4-methylpiperazin-1-yl)-[1,2,4]triazolo[4,3-b]pyridazin-3-yl]propanamide 20l (LIT-TB031)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 19.8 mg, 0.0419 mmol), KI (1 eq., 7.73 mg, 0.0466 mmol) and cyclohexylmethyl 4-methylbenzene-1-sulfonate (1.1 eq., 13.7 mg, 0.0512 mmol) and K₂CO₃ (5 eq., 32.2 mg, 0.233 mmol) in DMF (0.3 ml) at 85° C. overnight. The crude was evaporated and purified by reverse phase chromatography (H₂O/MeOH), salified and lyophilized to yield 20l as a yellowish solid (m=4.1 mg, yield=16%).

¹H NMR (500 MHz, Methanol-d₄) δ 7.92 (d, J=10.2 Hz, 1H), 7.38 (d, J=10.2 Hz, 1H), 3.74-3.61 (m, 5H), 3.38 (t, J=7.6 Hz, 2H), 2.87 (d, J=11.8 Hz, 2H), 2.80 (t, J=7.6 Hz, 2H), 2.64-2.62 (m, 4H), 2.40 (s, 3H), 2.18 (d, J=6.8 Hz, 2H), 2.06 (t, J=11.6 Hz, 2H), 1.84-1.70 (m, 7H), 1.35-1.21 (m, 3H), 1.37-1.17 (m, 3H), 0.98-0.90 (m, 2H). ¹³C NMR (126 MHz, Methanol-d₄) δ 173.2, 156.8, 150.1, 144.0, 124.7, 116.6, 66.9, 55.4, 54.0, 48.1, 46.4, 46.1, 36.4, 33.2, 33.2, 32.3, 27.7, 27.2, 21.2.

LC-MS (ESI) [M+H]⁺=469.24

### N-{1-[(5-methyl-1H-imidazol-4-yl)methyl]piperidin-4-yl}-3-[6-(4-methylpiperazin-1-yl)-[1,2,4] triazolo [4,3-b]pyridazin-3-yl]propanamide 20m (LIT-TB032)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 22 mg, 0.0466 mmol), KI (1 eq., 7.73 mg, 0.0466 mmol) and 4-(chloromethyl)-5-methyl-1H-imidazole (1.1 eq., 6.69 mg, 0.0512 mmol) and K₂CO₃ (5 eq., 32.2 mg, 0.233 mmol) in DMF (0.5 ml) at 85° C. for 5 h. The crude was evaporated and purified by reverse phase chromatography (H₂O/MeOH), salified and lyophilized to yield 20m as a yellowish solid (m=7.8 mg, yield=30%).

¹H NMR (500 MHz, Methanol-d₄) δ 7.95 (d, J=10.2 Hz, 1H), 7.57 (s, 1H), 7.40 (d, J=10.2 Hz, 1H), 3.74-3.72 (m, 4H), 3.70-3.63 (m, 1H), 3.55 (s, 2H), 3.42-3.39 (m, 2H), 2.93 (d, J=11.6 Hz, 2H), 2.82 (t, J=7.6 Hz, 2H), 2.67-2.65 (m, 4H), 2.43 (s, 3H), 2.27 (s, 3H), 2.26-2.21 (m, 2H), 1.87 (dd, J=13.2, 3.8 Hz, 2H), 1.57-1.50 (m, 2H). ¹³C NMR (126 MHz, Methanol-d₄) δ 173.2, 156.8, 150.1, 143.9, 134.8, 124.7, 116.6, 55.4, 52.9, 47.8, 46.4, 46.1, 33.2, 32.3, 21.2

LC-MS (ESI) [M+H]⁺=467.21

### N-{1-[(2,4-difluorophenyl)methyl]piperidin-4-yl}-3-[6-(4-methylpiperazin-1-yl)-[1,2,4]triazolo[4,3-b]pyridazin-3-yl]propanamide 20n (LIT-TB040)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 26 mg, 0.055 mmol), 2,4-difluorobenzyl bromide (1.1 eq., 12.5 mg, 7.78 μL, 0.0605 mmol) and K₂CO₃ (5 eq., 38 mg, 0.275 mmol) in DMF (0.3 ml). The crude was evaporated and purified by reverse phase chromatography (H₂O/MeOH), salified and lyophilized to yield 20n as a yellowish solid (m=25.6 mg, yield=81%).

¹H NMR (500 MHz, Methanol-d₄) δ 7.93 (d, J=10.2 Hz, 1H), 7.49-7.44 (m, 1H), 7.38 (d, J=10.2 Hz, 1H), 7.01-6.95 (m, 2H), 3.72-3.70 (m, 4H), 3.70-3.63 (m, 1H), 3.60 (s, 2H), 3.40-3.37 (m, 2H), 2.88 (d, J=11.9 Hz, 2H), 2.80 (t, J=7.6 Hz, 2H), 2.65-2.63 (m, 4H), 2.41 (s, 3H), 2.23-2.18 (m, 2H), 1.88-1.80 (m, 2H), 1.56-1.48 (m, 2H). ¹³C NMR (126 MHz, Methanol-d₄) δ 173.2, 163.9 (dd, J=247.0, 12.0 Hz), 162.9 (dd, J=248.0, 12.5 Hz), 156.8, 150.1, 143.9, 134.3 (dd, J=9.6, 5.9 Hz), 124.7, 121.5 (dd, J=14.7, 3.7 Hz), 116.5, 112.1 (dd, J=21.6, 3.8 Hz), 104.4 (dd, J=26.8, 25.7 Hz), 55.5, 55.4, 53.0, 47.8, 46.5, 46.1, 33.2, 32.4, 21.2. ¹⁹F NMR (376 MHz, Methanol-d₄) δ−113.2, −114.8.

LC-MS (ESI) [M+H]+=499.21

### N-{1-[(4-fluoro-2-methylphenyl)methyl]piperidin-4-yl}-3-[6-(4-methylpiperazin-1-yl)-[1,2,4]triazolo[4,3-b]pyridazin-3-yl]propanamide (LIT-TB044)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 24.7 mg, 0.0523 mmol), 1-(bromomethyl)-4-fluoro-2-methylbenzene (1.1 eq., 11.7 mg, 8.02 μL, 0.0575 mmol) and K₂CO₃ (5 eq., 36.1 mg, 0.261 mmol) in DMF (0.4 ml). The crude was evaporated and purified by reverse phase chromatography (H₂O/MeOH), salified and lyophilized to yield 20o as a yellowish solid (m=14.8 mg, yield=50%).

¹H NMR (500 MHz, Methanol-d₄) δ 7.87 (d, J=10.2 Hz, 1H), 7.33 (d, J=10.2 Hz, 1H), 7.21 (dd, J=8.4, 6.0 Hz, 1H), 6.88 (dd, J=9.9, 2.7 Hz, 1H), 6.83 (td, J=8.5, 2.8 Hz, 1H), 3.67-3.60 (m, 5H), 3.42 (s, 2H), 3.35-3.31 (m, 2H), 2.80 (d, J=11.6 Hz, 2H), 2.75 (t, J=7.6 Hz, 2H), 2.58 (t, J=5.1 Hz, 4H), 2.35 (s, 3H), 2.35 (s, 3H), 2.09 (td, J=11.7, 2.5 Hz, 2H), 1.76 (dd, J=13.5, 4.0 Hz, 2H), 1.42 (qd, J=11.6, 3.8 Hz, 2H). ¹³C NMR (126 MHz, Methanol-d₄) δ 173.2, 163.3 (d, J=243.4 Hz), 156.8, 150.1, 143.9, 141.4 (d, J=7.7 Hz), 133.4 (d, J=2.9 Hz), 132.8 (d, J=8.3 Hz), 124.7, 117.7 (d, J=21.0 Hz), 116.6, 112.9 (d, J=20.9 Hz), 60.8, 55.4, 53.4, 48.1, 46.4, 46.1, 33.2, 32.6, 21.2, 19.5. ¹⁹F NMR (471 MHz, Methanol-d₄) δ−118.5.

LC-MS (ESI) [M+H]⁺=495.28

### N-{1-[(4-methoxy-2-methylphenyl)methyl]piperidin-4-yl}-3-[6-(4-methylpiperazin-1-yl)-[1,2,4]triazolo[4,3-b]pyridazin-3-yl]propanamide dihydrochloride 20p (LIT-TB045)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 25 mg, 0.0529 mmol), 1-(bromomethyl)-4-methoxy-2-methylbenzene (1.2 eq., 13.7 mg, 0.0635 mmol) and K₂CO₃ (5 eq., 36.6 mg, 0.265 mmol) in DMF (0.4 ml). The crude was evaporated and purified by reverse phase chromatography (H₂O/MeOH), salified and lyophilized to yield 20p as a yellowish solid (m=11.5 mg, yield=38%).

¹H NMR (500 MHz, Methanol-d₄) δ 7.97 (d, J=10.1 Hz, 1H), 7.42 (d, J=10.2 Hz, 1H), 7.21 (d, J=8.4 Hz, 1H), 6.82 (d, J=2.6 Hz, 1H), 6.78 (dd, J=8.3, 2.7 Hz, 1H), 3.86 (s, 3H), 3.78-3.70 (m, 5H), 3.50 (s, 2H), 3.46-3.41 (m, 2H), 2.92 (d, J=11.8 Hz, 2H), 2.85 (t, J=7.6 Hz, 2H), 2.69 (t, J=5.1 Hz, 4H), 2.46 (s, 3H), 2.43 (s, 3H), 2.22-2.16 (m, 2H), 1.90-1.84 (m, 2H), 1.53 (qd, J=11.5, 3.7 Hz, 2H). ¹³C NMR (126 MHz, Methanol-d₄) δ 173.2, 160.3, 156.8, 150.1, 143.9, 140.1, 132.5, 129.4, 124.7, 116.9, 116.6, 111.6, 60.9, 55.6, 55.4, 53.4, 48.2, 46.4, 46.1, 33.3, 32.6, 21.2, 19.7.

LC-MS (ESI) [M+H]⁺=507.31

### N-{1-[(2-fluoro-4-methoxyphenyl)methyl]piperidin-4-yl}-3-[6-(4-methylpiperazin-1-yl)-[1,2,4]triazolo[4,3-b]pyridazin-3-yl]propanamide dihydrochloride 20q (LIT-TB046)

General procedure B for the synthesis of 20a was followed using tert-butyl 4-{3-[6-(4-methylpiperazin-1-yl)-[1,2,4]triazolo[4,3-b]pyridazin-3-yl]propanamido}piperidine-1-carboxylate 18a (1 eq., 26 mg, 0.055 mmol), 1-(bromomethyl)-2-fluoro-4-methoxybenzene (1.4 eq., 16.9 mg, 0.077 mmol) and K₂CO₃ (5 eq., 38 mg, 0.275 mmol) in DMF (0.4 ml). The crude was evaporated and purified by reverse phase chromatography (H₂O/MeOH), salified and lyophilized to yield 20q as a yellowish solid (m=16.5 mg, yield=51%).

¹H NMR (400 MHz, Methanol-d₄) δ 7.89 (dd, J=10.3, 2.9 Hz, 1H), 7.34 (dd, J=10.5, 2.8 Hz, 1H), 7.27 (dd, J=10.0, 7.5 Hz, 1H), 6.73 (d, J=8.6 Hz, 1H), 6.68 (d, J=12.1 Hz, 1H), 3.79 (s, 3H), 3.69-3.57 (m, 5H), 3.52 (s, 2H), 3.36-3.33 (m, 2H), 2.85 (d, J=11.6 Hz, 2H), 2.75 (t, J=7.8 Hz, 2H), 2.62-2.58 (m, 4H), 2.36 (s, 3H), 2.14 (t, J=11.8 Hz, 2H), 1.79 (d, J=12.8 Hz, 2H), 1.46 (q, J=12.1 Hz, 2H). ¹³C NMR (101 MHz, Methanol-d₄) δ 173.2, 163.5 (d, J=244.9 Hz), 162.2 (d, J=11.2 Hz), 156.8, 150.1, 143.9, 133.9 (d, J=6.2 Hz), 124.7, 116.56, 116.55 (d, J=15.6 Hz), 110.9 (d, J=2.9 Hz), 102.2 (d, J=26.6 Hz), 56.1, 55.6, 55.4, 52.8, 47.8, 46.4, 46.1, 33.2, 32.3, 21.2. ¹⁹F NMR (376 MHz, Methanol-d₄) δ−116.9.

LC-MS (ESI) [M+H]⁺=511.26

### Example 5: 3-[6-(4-methylpiperazin-1-yl)-[1,2,4]triazolo[4,3-b]pyridazin-3-yl]-N-[1-(1,3-oxazol-4-ylmethyl)piperidin-4-yl]propanamide 20r (LIT-TB050)

Tert-butyl 4-{3-[6-(4-methylpiperazin-1-yl)-[1,2,4]triazolo[4,3-b]pyridazin-3-yl]propanamido} piperidi-ne-1-carboxylate 18a (18.6 mg, 0.039 mmol) was solubilized in DCM (0.3 mL). TFA (10 eq., 41.5 mg, 27 μL, 0.364 mmol) was added and the reaction mixture was stirred at r.t. for 2 h. The crude was evaporated, and then co-evaporated twice with DCM/heptane. After drying, the crude was taken in a saturated solution of K₂CO₃ and extracted twice with DCM. The organic phases were dried on Na₂SO₄, filtered and evaporated. The crude (13 mg, 0.035 mmol) was used for the next step without further purification.

3-[6-(4-methylpiperazin-1-yl)-[1,2,4]triazolo[4,3-b]pyridazin-3-yl]-N-(piperidin-4-yl)propanamide 19 (1 eq., 13 mg, 0.0349 mmol) was solubilized in dry MeOH (0.5 ml) under argon. 1,3-oxazole-4-carbaldehyde (2 eq., 6.78 mg, 0.0698 mmol) was added and the reaction mixture was stirred at r.t. for 10 min. NaBH(OAc)₃ (2 eq., 15.6 mg, 0.0698 mmol) was solubilized in dry MeOH (0.5 ml) and added to the reaction mixture. The reaction was stirred at r.t. for 40 h. Water was added and the crude was directly purified by reverse phase chromatography (H₂O/MeOH), salified with aqueous HCl (2M) and lyophilized to yield 20r as a white solid (m=3.7 mg, yield=20%).

¹H NMR (500 MHz, Methanol-d4) δ 8.16 (d, J=0.9 Hz, 1H), 7.90 (d, J=10.2 Hz, 1H), 7.86 (d, J=0.9 Hz, 1H), 7.36 (d, J=10.2 Hz, 1H), 3.69-3.65 (m, 4H), 3.65-3.58 (m, 1H), 3.52 (s, 2H), 3.37-3.33 (m, 2H), 2.90 (d, J=11.8 Hz, 2H), 2.76 (t, J=7.6 Hz, 2H), 2.60 (t, J=5.1 Hz, 4H), 2.37 (s, 3H), 2.22-2.12 (m, 2H), 1.81 (dd, J=13.4, 3.8 Hz, 2H), 1.52-1.44 (m, 2H). ¹³C NMR (126 MHz, Methanol-d4) δ 173.2, 156.8, 153.4, 150.1, 144.0, 139.0, 137.2, 124.7, 116.6, 55.4, 53.8, 53.1, 47.8, 46.4, 46.1, 33.2, 32.3, 21.2.

LC-MS (ESI) [M+H]+=454.24

General Procedure C

In the general procedure C, diacylation of hydrazino-pyridazine 5 is followed by cyclisation under acid conditions of 22 to afford the ethyl propanoate triazolopyridazine 23 (Scheme 5). Reaction with secondary amines yielded the triazolopyridazines 24 with various amine substitutions in position 6. Hydrolysis of the carboxylic ester and coupling to primary amines 25 yielded the final analogues 26 with another diversity point on the 6-membered aliphatic ring (scheme 5)

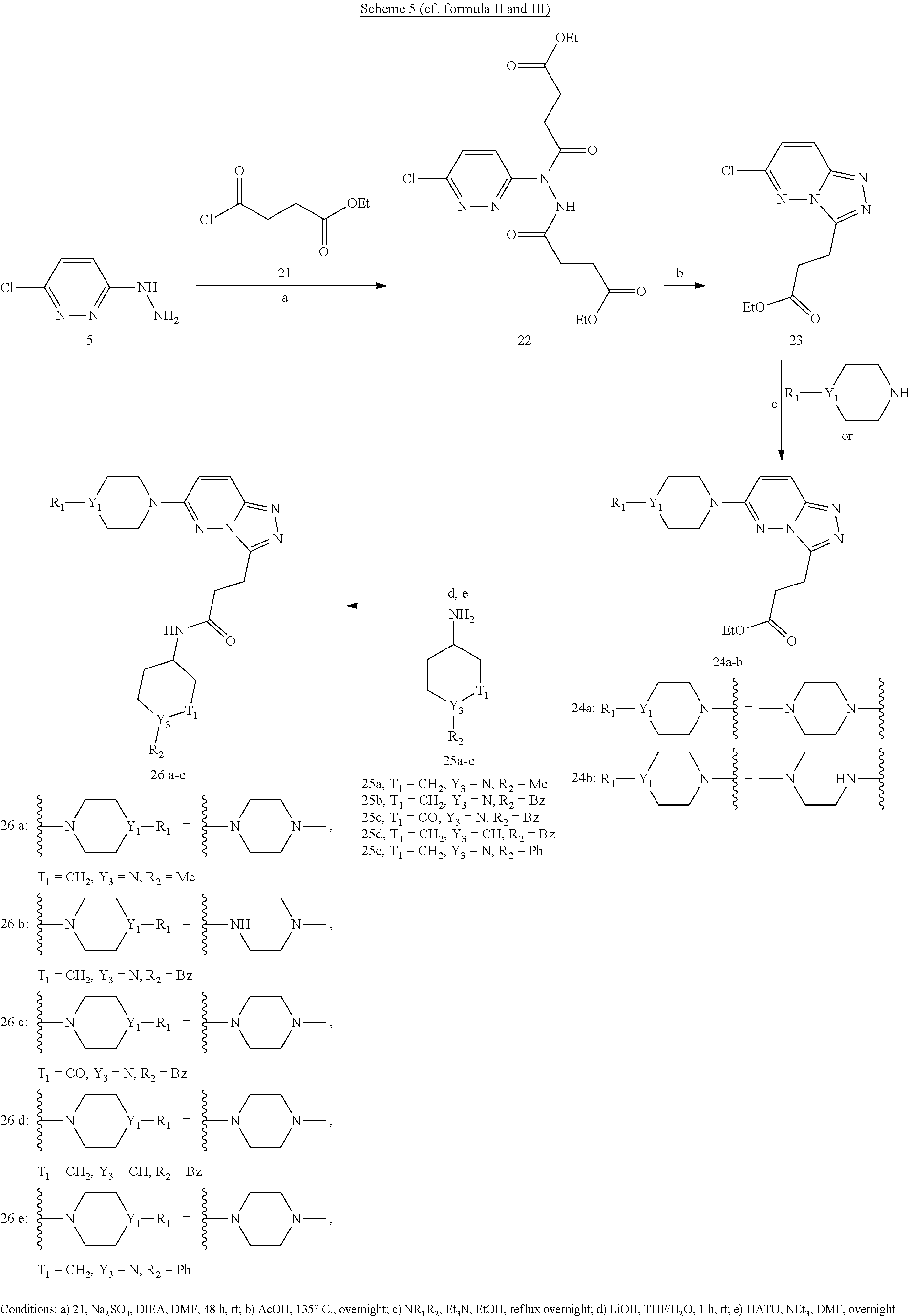

### Ethyl 4-[2-(6-chloropyridazin-3-yl)-2-(4-ethoxy-4-oxo-butanoyl)hydrazino]-4-oxo-butanoate 22

3-Chloro-6-hydrazinylpyridazine 5 (1 eq., 600 mg, 4.15 mmol) was solubilized in dry DMF (10 ml). Na₂SO₄ (50 mg) and DIEA (2.2 eq., 1180 mg, 1.51 mL, 9.13 mmol) were added and the reaction mixture was cooled to O ° C. and stirred for 15 min. Ethyl succinyl chloride 21 (1.2 eq., 819 mg, 0.708 mL, 4.98 mmol) was then added dropwise and the reaction mixture was stirred over the weekend at r.t. DMF was evaporated and the crude was purified by silica gel chromatography (EtOAc/heptane, 1/1, 5/1 to 1/0) to yield as a white solid (m=1 g, yield=61%).

¹H NMR (400 MHz, Methanol-d₄) δ 7.51 (d, J=9.4 Hz, 1H), 7.15 (d, J=9.4 Hz, 1H), 4.15 (qd, J=7.1, 6.0 Hz, 4H), 2.72-2.54 (m, 8H), 1.26 (td, J=7.1, 1.9 Hz, 6H). ¹³C NMR (101 MHz, Methanol-d₄) δ 174.4, 174.3, 174.1, 173.3, 161.6, 149.6, 131.3, 118.2, 61.8, 61.7, 30.0, 29.9, 29.32, 29.28, 14.48, 14.46.

### Ethyl 3-{6-chloro-[1,2,4]triazolo[4,3-b]pyridazin-3-yl}propanoate 23

Ethyl 4-[2-(6-chloropyridazin-3-yl)-2-(4-ethoxy-4-oxo-butanoyl)hydrazino]-4-oxo-butanoate 22 (1 eq., 960 mg, 3.52 mmol) was solubilized in acetic acid (38.6 eq., 8157 mg, 7.78 mL, 135 mmol) and the reaction was heated at 135° C. overnight. The crude was cooled to r.t. and evaporated. The crude was purified by silica gel chromatography (heptane/EtOAc; 1/1, 1/5 to 0/1) to yield compound 23 as a white solid (m=586 mg, yield=96%).

¹H NMR (400 MHz, Methanol-d₄) δ 8.26 (d, J=9.7 Hz, 1H), 7.45 (d, J=9.7 Hz, 1H), 4.16 (q, J=7.1 Hz, 2H), 3.47 (t, J=7.3 Hz, 2H), 3.04 (t, J=7.3 Hz, 2H), 1.26 (t, J=7.1 Hz, 3H). ¹³C NMR (101 MHz, Methanol-d₄) δ 173.5, 151.2, 150.6, 144.6, 127.3, 124.6, 61.9, 31.2, 20.4, 14.4.

### Ethyl 3-[6-(4-methylpiperazin-1-yl)-[1,2,4]triazolo[4,3-b]pyridazin-3-yl]propanoate 24a

Ethyl 3-{6-chloro-[1,2,4]triazolo[4,3-b]pyridazin-3-yl}propanoate 23 (1 eq., 586 mg, 2.3 mmol) was solubilised in EtOH (2.5 ml). 1-methylpiperazine (2 eq., 460 mg, 0.51 mL, 4.6 mmol) and Et₃N (2 eq., 465 mg, 0.64 mL, 4.6 mmol) were added and the reaction was heated at reflux overnight. The crude was cooled to r.t. and evaporated. The crude was purified by silica gel chromatography (EtOAc/MeOH/Et₃N; 9/1/0.5 to 7/1/0.5) to yield 24a as a pale yellow solid (m=728 mg, yield=99%).

¹H NMR (400 MHz, Methanol-d₄) δ 7.97 (d, J=10.2 Hz, 1H), 7.39 (d, J=10.2 Hz, 1H), 4.12 (q, J=7.1 Hz, 2H), 3.90-3.85 (m, 4H), 3.36 (t, J=7.4 Hz, 2H), 3.24-3.19 (m, 4H), 2.97 (t, J=7.4 Hz, 2H), 2.80 (s, 3H), 1.21 (t, J=7.1 Hz, 3H). ¹³C NMR (101 MHz, Methanol-d₄) δ 173.7, 156.4, 149.9, 144.0, 125.3, 116.5, 61.9, 54.3, 31.3, 20.5, 14.4.

### Example 6: 3-[6-(4-methylpiperazin-1-yl)-[1,2,4]triazolo[4,3-b]pyridazin-3-yl]-N-(1-methyl-piperidin-4-yl)propanamide 26a (LIT-TB016)

Ethyl 3-[6-(4-methylpiperazin-1-yl)-[1,2,4]triazolo[4,3-b]pyridazin-3-yl]propanoate 24a (1 eq., 30 mg, 0.0942 mmol) was diluted in a mixture THF/H₂O (1/1; 6 ml). LiOH (5 eq., 19.8 mg, 0.471 mmol) was added and the reaction mixture was stirred at r.t. for 1 h. The crude was acidified with HCl (2M), evaporated and diluted in dry DMF (0.5 ml). HATU (2.5 eq., 89.6 mg, 0.236 mmol) and Et₃N (2.5 eq., 23.8 mg, 32.7 μL, 0.236 mmol) were added and the reaction mixture was stirred at r.t. for 15 min. 1-methylpiperidin-4-amine 25a (1.2 eq., 13.3 mg, 14.6 μL, 0.113 mmol) was then added and the reaction mixture was stirred overnight at r.t. The crude was directly purified by reverse phase chromatography (MeOH/H₂O) to yield sticky oil. A second purification was performed to yield the desired compound. The product was evaporated and diluted in MeOH. 2M-HCl in Et₂O (excess) was added and the reaction was stirred at r.t. for 1.5 h. The mixture was evaporated, diluted in water and lyophilized to give 26a as a white solid (m=2.9 mg, yield=7%).

¹H NMR (500 MHz, Methanol-d₄) δ 7.88 (d, J=10.2 Hz, 1H), 7.34 (d, J=10.2 Hz, 1H), 3.68-3.63 (m, 5H), 3.33 (t, J=7.5 Hz, 2H), 2.93-2.85 (m, 2H), 2.76 (t, J=7.5, 2H), 2.61-2.57 (m, 4H), 2.36 (s, 3H), 2.34 (s, 3H), 2.25 (t, J=11.8 Hz, 2H), 1.88-1.83 (m, 2H), 1.54-1.47 (m, 2H). ¹³C NMR (126 MHz, Methanol-d₄) δ 173.4, 156.8, 150.1, 144.0, 124.7, 116.6, 68.9, 55.4, 46.4, 46.1, 45.8, 33.1, 31.9, 26.5, 21.1.

LC-MS (ESI) [M+H]⁺=387.17

### N-(1-benzyl-4-piperidyl)-3-[6-[2-(dimethylamino)ethylamino]-[1,2,4]triazolo[4,3-b]pyridazin-3-yl]propanamide 26b (LIT-TB051)

Ethyl 3-[6-[2-(dimethylamino)ethylamino]-[1,2,4]triazolo[4,3-b]pyridazin-3-yl]propanoate 24b (1 eq., 18 mg, 0.0588 mmol) was diluted in a mixture THF/H₂O (1/1; 6 ml). LiOH (5 eq., 12.3 mg, 8.62 μL, 0.294 mmol) was added and the reaction mixture was stirred at r.t. for 1 h. The crude was acidified with HCl (2M), evaporated and diluted in dry DMF (0.5 ml). Sulfate was added to the mixture and stirred for 5 min. HATU (1.2 eq., 26.8 mg, 0.0705 mmol) and Et₃N (2.5 eq., 14.9 mg, 20.4 μL, 0.147 mmol) were added and the reaction mixture was stirred at r.t. for 15 min. 4-amino-1-benzylpiperidine 25b (1.5 eq., 16.8 mg, 18 p, 0.0881 mmol) was then added and the reaction mixture was stirred 3 h at 60° C. The crude was filtered over a pad of celite and washed with MeOH. The filtrate was evaporated and purified by reverse phase chromatography (MeOH/H₂O), salified using aqueous HCl (2M), and lyophilized to yield 26b as a white solid (m=14.3 mg, yield=46%).

¹H NMR (400 MHz, Methanol-d₄) δ 7.74 (d, J=9.9 Hz, 1H), 7.34-7.24 (m, 5H), 6.81 (d, J=9.9 Hz, 1H), 3.68-3.60 (m, 1H), 3.58-3.53 (m, 4H), 3.35-3.29 (m, 2H), 2.87 (d, J=11.7 Hz, 2H), 2.78-2.73 (m, 4H), 2.41 (s, 6H), 2.18-2.12 (m, 2H), 1.81 (dd, J=13.4, 3.9 Hz, 2H), 1.55-1.42 (m, 2H). ¹³C NMR (101 MHz, Methanol-d₄) δ 173.2, 155.8, 149.9, 144.3, 138.2, 130.8, 129.4, 128.6, 124.0, 119.4, 63.9, 58.2, 53.2, 47.8, 45.4, 39.7, 33.2, 32.2, 21.1.

LC-MS (ESI) [M+H]⁺=451.26

### N-(1-benzyl-2-oxopiperidin-4-yl)-3-[6-(4-methylpiperazin-1-yl)-[1,2,4]triazolo[4,3-b]pyridazin-3-yl]propanamide 26c (LIT-TB033)

The general procedure C for the synthesis of 26a was followed using ethyl 3-[6-(4-methylpiperazin-1-yl)-[1,2,4]triazolo[4,3-b]pyridazin-3-yl]propanoate 11a (1.5 eq., 54.9 mg, 0.173 mmol) and LiOH (5 eq., 24.1 mg, 0.575 mmol) in THF/H2O (1/1; 6 ml). The crude was treated with HATU (1.2 eq., 52.5 mg, 0.138 mmol), Et₃N (5 eq., 58.2 mg, 80 μL, 0.575 mmol), and 4-amino-1-benzylpiperidin-2-one 25c (1 eq., 23.5 mg, 0.115 mmol) in dry DMF (1 ml). The crude was directly purified by reverse phase chromatography (MeOH/H₂O). A semi-preparative chromatography (MeOH/H₂O+0.05% HCl) was performed to isolate the product. The compound was salified and lyophilized to yield 26c as a yellowish solid (m=8.5 mg, yield=14%).

¹H NMR (500 MHz, Methanol-d₄) δ 7.78 (d, J=10.2 Hz, 1H), 7.26-7.20 (m, 3H), 7.17-7.14 (m, 3H), 4.56-4.41 (m, 2H), 4.00 (tdd, J=9.1, 5.7, 3.3 Hz, 1H), 3.57-3.55 (m, 4H), 3.27-3.16 (m, 4H), 2.68 (t, J=7.5 Hz, 2H), 2.62 (ddd, J=17.4, 5.7, 1.6 Hz, 1H), 2.51-2.49 (m, 4H), 2.26 (s, 3H), 2.23 (dd, J=17.9, 9.2 Hz, 1H), 1.89 (ddt, J=13.0, 4.8, 3.1 Hz, 1H), 1.68-1.59 (m, 1H). ¹³C NMR (126 MHz, Methanol-d₄) δ 173.6, 170.5, 156.8, 150.0, 144.0, 138.1, 129.7, 129.0, 128.6, 124.7, 116.6, 55.4, 50.9, 46.4, 46.1, 45.5, 45.1, 38.5, 33.0, 29.2, 21.0.

LC-MS (ESI) [M+H]⁺=477.19

### N-(4-benzylcyclohexyl)-3-[6-(4-methylpiperazin-1-yl)-[1,2,4]triazolo[4,3-b]pyridazin-3-yl]propanamide 26d (LIT-TB034)

The general procedure C for the synthesis of 26a was followed using ethyl 3-[6-(4-methylpiperazin-1-yl)-[1,2,4]triazolo[4,3-b]pyridazin-3-yl]propanoate 24a (1.5 eq., 50.2 mg, 0.158 mmol) and LiOH (5 eq., 22.1 mg, 0.526 mmol) in THF/H₂O (1/1; 6 ml). The crude of was treated with HATU (1.2 eq., 48 mg, 0.126 mmol), Et₃N (5 eq., 53.2 mg, 73 μL, 0.526 mmol), and 4-benzylcyclohexan-1-amine 25d (1 eq., 19.9 mg, 0.105 mmol) in dry DMF (1 ml). The crude was directly purified by reverse phase chromatography (MeOH/H₂O). A semi-preparative chromatography (MeOH/H₂O+0.05% HCl) was performed to isolate the product. The compound was salified and lyophilized to yield 26d as a light yellowish solid (m=11.3 mg, yield=22%).

¹H NMR (500 MHz, Methanol-d₄) δ 8.26 (d, J=9.7 Hz, 1H), 7.93 (d, J=9.8 Hz, 1H), 7.22-7.19 (m, 2H), 7.14-7.07 (m, 3H), 4.59 (d, J=14.2 Hz, 2H), 3.66 (d, J=11.5 Hz, 2H), 3.60-3.48 (m, 3H), 3.43 (t, J=6.5 Hz, 2H), 3.35-3.28 (m, 2H), 2.96 (s, 3H), 2.85-2.82 (m, 2H), 2.46 (d, J=7.0 Hz, 2H), 1.81 (d, J=9.3 Hz, 2H), 1.70 (d, J=11.0 Hz, 2H), 1.47 (ddt, J=11.3, 7.7, 3.8 Hz, 1H), 1.19-1.11 (m, 2H), 1.07-0.96 (m, 2H). ¹³C NMR (126 MHz, Methanol-d₄) δ 172.1, 157.5, 150.5, 142.1, 141.0, 130.1, 129.2, 126.8, 122.6, 122.6, 53.8, 50.2, 44.4, 44.1, 43.7, 40.3, 33.5, 32.7, 31.9, 20.7.

LC-MS (ESI) [M+H]⁺=462.20

### 3-[6-(4-methylpiperazin-1-yl)-[1,2,4]triazolo[4,3-b]pyridazin-3-yl]-N-(1-phenylpiperidin-4-yl)propanamide 26e (LIT-TB035)

The general procedure C for the synthesis of 26a was followed using ethyl 3-[6-(4-methylpiperazin-1-yl)-[1,2,4]triazolo[4,3-b]pyridazin-3-yl]propanoate 24a (1.5 eq., 60 mg, 0.188 mmol) and LiOH (1.5 eq., 60 mg, 0.188 mmol) in THF/H₂O (1/1; 6 ml). The crude was treated with HATU (1.2 eq., 57.3 mg, 0.151 mmol), Et₃N (5 eq., 63.6 mg, 87.3 μL, 0.628 mmol), and 1-phenylpiperidin-4-amine 1-phenylpiperidin-4-amine 25e (1 eq., 22.1 mg, 0.126 mmol; CAS 63921-23-3) in dry DMF (1 ml). The crude was directly purified by reverse phase chromatography (MeOH/H₂O). The compound was salified and lyophilized to yield 26e as a light yellowish solid (m=20.9 mg, yield=34%).

¹H NMR (500 MHz, Methanol-d₄) δ 7.88 (d, J=10.1 Hz, 1H), 7.34 (d, J=10.2 Hz, 1H), 7.23-7.17 (m, 2H), 6.99-6.94 (m, 2H), 6.81 (tt, J=7.3, 1.1 Hz, 1H), 3.77 (tt, J=10.8, 4.2 Hz, 1H), 3.69-3.63 (m, 4H), 3.61-3.56 (m, 2H), 3.35 (t, J=7.6 Hz, 2H), 2.82-2.74 (m, 4H), 2.59 (t, J=5.1 Hz, 4H), 2.35 (s, 3H), 1.92-1.88 (m, 2H), 1.61-1.53 (m, 2H), NH. ¹³C NMR (126 MHz, Methanol-d₄) δ 173.3, 156.8, 152.8, 150.1, 144.0, 130.0, 124.7, 121.1, 118.2, 116.6, 55.4, 50.2, 48.0, 46.4, 46.1, 33.2, 32.5, 21.2.

LC-MS (ESI) [M+H]1=449.17

General Procedure D for the Preparation of 3-fluoro-4-aminopiperidine Analogues of LIT-TB001

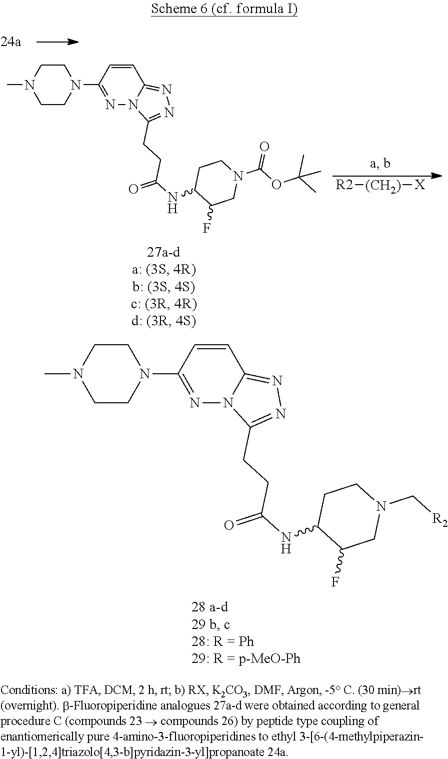

### tert-butyl (3S,4R)-3-fluoro-4-{3-[6-(4-methylpiperazin-1-yl)-[1,2,4]triazolo[4,3-b]pyridazin-3-yl]propanamido}piperidine-1-carboxylate 27a

¹H NMR (500 MHz, Methanol-d₄) δ 7.90 (d, J=10.2 Hz, 1H), 7.36 (d, J=10.2 Hz, 1H), 4.64 (d, J=48.9 Hz, 1H), 4.35 (s, 1H), 4.14 (d, J=12.6 Hz, 1H), 4.00 (dddd, J=30.8, 12.3, 4.9, 2.2 Hz, 1H), 3.67 (dd, J=6.2, 4.1 Hz, 4H), 3.39-3.34 (m, 2H), 2.83 (t, J=7.6 Hz, 2H), 2.61 (t, J=5.1 Hz, 4H), 2.38 (s, 3H), 1.74 (qd, J=12.7, 4.5 Hz, 1H), 1.62 (ddd, J=10.1, 5.2, 2.6 Hz, 1H), 1.46 (s, 9H), 1.35-1.29 (m, 2H), NH (not visible). ¹³C NMR (126 MHz, Methanol-d₄) δ 173.5, 156.9, 156.8, 150.0, 144.0, 124.7, 116.6, 88.5 (d, J=177.3 Hz), 81.4, 55.4, 50.1 (d, J=18.9 Hz), 46.4, 46.1, 33.0, 32.9, 28.6, 23.7, 21.1, 14.4.

¹⁹F NMR (471 MHz, Methanol-d₄) δ−205.7.

### Example 7: N-[(3S,4R)-1-benzyl-3-fluoropiperidin-4-yl]-3-[6-(4-methylpiperazin-1-yl)-[1,2,4] triazolo[4,3-b]pyridazin-3-yl]propanamide 28a (LIT-TB047)

Tert-butyl (3S,4R)-3-fluoro-4-{3-[6-(4-methylpiperazin-1-yl)-[1,2,4]triazolo[4,3-b]pyridazin-3-yl]propanamido}piperidine-1-carboxylate 27a (1 eq., 30.4 mg, 0.062 mmol) was solubilized in DCM (0.7 mL). TFA (10 eq., 70.7 mg, 46 μL, 0.62 mmol) was added and the reaction mixture was stirred at r.t. for 2 h. The crude was evaporated, then co-evaporated with DCM/heptane (3×). After drying, the crude was solubilized in dry DMF under Argon. K₂CO₃ (5 eq., 42.8 mg, 0.31 mmol) was added and the reaction mixture was stirred at −5° C. for 30 min. The benzylbromide (1.1 eq., 11.7 mg, 8.15 μL, 0.0682 mmol) was added, and the mixture was stirred at −5° C. for 0.5 h then at r.t. overnight. Water (few drops) was added and the crude was directly purified by reverse phase chromatography (H₂O/MeOH), salified and lyophilized to yield the title compound 28a as a yellowish solid ((m=18.8 mg, yield=55%).

¹H NMR (400 MHz, Methanol-d₄) δ 7.88 (d, J=10.2 Hz, 1H), 7.37-7.22 (m, 6H), 4.61 (d, J=49.3 Hz, 1H), 3.84 (dd, J=30.4, 12.2 Hz, 1H), 3.65 (t, J=4.8 Hz, 4H), 3.63-3.48 (m, 2H), 3.37-3.31 (m, 2H), 3.11 (t, J=11.8 Hz, 1H), 2.90 (d, J=11.7 Hz, 1H), 2.85-2.77 (m, 2H), 2.59 (t, J=4.8 Hz, 4H), 2.36 (s, 3H), 2.29-2.15 (m, 2H), 1.89 (q, J=13.0, 12.5 Hz, 1H), 1.63 (d, J=13.0 Hz, 1H). ¹³C NMR (101 MHz, Methanol-d₄) δ 173.5, 156.8, 150.0, 144.0, 138.3, 130.5, 129.3, 128.4, 124.7, 116.6, 89.0 (d, J=177.1 Hz), 63.3, 56.3 (d, J=18.9 Hz), 55.4, 52.7, 50.0 (d, J=18.5 Hz), 46.4, 46.1, 32.9, 27.0, 21.1. ¹⁹F NMR (376 MHz, Methanol-d₄) δ−201.6.

LC-MS (ESI) [M+H]⁺=481.25

### N-[(3S,4S)-1-benzyl-3-fluoropiperidin-4-yl]-3-[6-(4-methylpiperazin-1-yl)-[1,2,4]triazolo[4,3-b]pyridazin-3-yl]propanamide 28b (LIT-TB048)

General procedure D for the synthesis of 28a was followed using tert-butyl (3S,4S)-3-fluoro-4-{3-[6-(4-methylpiperazin-1-yl)-[1,2,4]triazolo[4,3-b]pyridazin-3-yl]propanamido}piperidine-1-carboxylate 27b (1 eq., 26 mg, 0.053 mmol), benzylbromide (1.1 eq., 9.97 mg, 6.97 μL, 0.0583 mmol) and K₂CO₃ (5 eq., 36.6 mg, 0.265 mmol) in DMF (0.5 ml). The crude was evaporated and purified by reverse phase chromatography (H₂O/MeOH), salified and lyophilized to yield 28b as a yellowish solid (m=13.0 mg, yield=44%).

¹H NMR (400 MHz, Methanol-d₄) δ 7.88 (d, J=10.3 Hz, 1H), 7.35-7.25 (m, 6H), 4.46-4.21 (m, 1H), 3.88-3.75 (m, 1H), 3.65 (t, J=4.9 Hz, 4H), 3.61-3.53 (m, 2H), 3.37-3.33 (m, 2H), 3.10 (dd, J=11.0, 5.7 Hz, 1H), 2.82-2.76 (m, 3H), 2.59 (t, J=4.9 Hz, 4H), 2.36 (s, 3H), 2.16-2.06 (m, 2H), 1.89 (d, J=12.5 Hz, 1H), 1.46 (q, J=11.7 Hz, 1H). ¹³C NMR (101 MHz, Methanol-d₄) δ 173.9, 156.8, 150.0, 144.0, 138.7, 130.4, 129.4, 128.5, 124.7, 116.6, 90.6 (d, J=177.8 Hz), 63.2, 57.1 (d, J=25.0 Hz), 55.4, 52.6 (d, J=18.4 Hz), 52.4, 46.4, 46.1, 33.3, 30.4 (d, J=6.9 Hz), 21.1. ¹⁹F NMR (376 MHz, Methanol-d₄) δ−189.7.

LC-MS (ESI) [M+H]⁺=481.25

### N-[(3R,4R)-1-benzyl-3-fluoropiperidin-4-yl]-3-[6-(4-methylpiperazin-1-yl)-[1,2,4]triazolo[4,3-b]pyridazin-3-yl]propanamide 28c (LIT-TB049)

General procedure D for the synthesis of 28a was followed using tert-butyl (3R,4R)-3-fluoro-4-{3-[6-(4-methylpiperazin-1-yl)-[1,2,4]triazolo[4,3-b]pyridazin-3-yl]propanamido}piperidine-1-carboxylate 17c (1 eq., 22.4 mg, 0.0457 mmol), benzylbromide (1.1 eq., 8.59 mg, 6.01 μL, 0.0502 mmol) and K₂CO₃ 5 eq., 31.6 mg, 0.228 mmol) in DMF (0.5 ml). The crude was evaporated and purified by reverse phase chromatography (H₂O/MeOH), salified and lyophilized to yield 28c as a yellowish solid (m=13.4 mg, yield=54%).

¹H NMR (500 MHz, Methanol-d₄) δ 7.88 (d, J=10.1 Hz, 1H), 7.36-7.25 (m, 6H), 4.34 (dtd, J=49.7, 9.4, 4.7 Hz, 1H), 3.80 (tdd, J=11.2, 9.2, 5.0 Hz, 1H), 3.68-3.64 (m, 4H), 3.61-3.53 (m, 2H), 3.38-3.34 (m, 2H), 3.13-3.06 (m, 1H), 2.82-2.75 (m, 3H), 2.59 (t, J=5.1 Hz, 4H), 2.36 (s, 3H), 2.16-2.08 (m, 2H), 1.89 (dtt, J=13.6, 5.8, 3.0 Hz, 1H), 1.46 (dtdd, J=12.9, 11.7, 4.2, 1.0 Hz, 1H). 13C NMR (126 MHz, Methanol-d₄) δ 173.9, 156.8, 150.0, 144.0, 138.6, 130.4, 129.4, 128.5, 124.7, 116.6, 90.6 (d, J=177.9 Hz), 63.2, 57.1 (d, J=25.0 Hz), 55.4, 52.6 (d, J=18.5 Hz), 52.4, 46.4, 46.1, 33.3, 30.4 (d, J=6.8 Hz), 21.1. ¹⁹F NMR (471 MHz, Methanol-d₄) δ−189.7.

LC-MS (ESI) [M+H]⁺=481.26

### N-[(3R,4S)-1-benzyl-3-fluoropiperidin-4-yl]-3-[6-(4-methylpiperazin-1-yl)-[1,2,4]triazolo[4,3-b]pyridazin-3-yl]propanamide 18d (LIT-TB054)

General procedure D for the synthesis of 28a was followed using tert-butyl (3R,4S)-3-fluoro-4-{3-[6-(4-methylpiperazin-1-yl)-[1,2,4]triazolo[4,3-b]pyridazin-3-yl]propanamido}piperidine-1-carboxylate 17d (1 eq., 24 mg, 0.0489 mmol), (1.1 eq., 9.2 mg, 6.44 μL, 0.0538 mmol) and K₂CO₃ (5 eq., 33.8 mg, 0.245 mmol) in DMF (0.5 ml). The crude was evaporated and purified by reverse phase chromatography (H₂O/MeOH), salified and lyophilized to yield 28d as a yellowish solid (m=13.4 mg, yield=49%).

¹H NMR (400 MHz, Methanol-d₄) δ 7.88 (d, J=10.2 Hz, 1H), 7.36-7.24 (m, 6H), 4.61 (ddd, J=49.3, 3.8, 2.1 Hz, 1H), 3.84 (dddd, J=30.2, 12.3, 5.0, 2.5 Hz, 1H), 3.66 (t, J=5.1 Hz, 4H), 3.55 (dd, J=42.3, 13.0 Hz, 2H), 3.37-3.33 (m, 2H), 3.15-3.08 (m, 1H), 2.93-2.88 (m, 1H), 2.82 (t, J=7.6 Hz, 2H), 2.59 (t, J=5.1 Hz, 4H), 2.36 (s, 3H), 2.31-2.14 (m, 2H), 1.94-1.84 (m, 1H), 1.63 (dd, J=13.0, 3.9 Hz, 1H). ¹³C NMR (101 MHz, Methanol-d₄) b 173.54, 156.78, 150.02, 143.95, 138.28, 130.55, 129.31, 128.43, 124.71, 116.57, 89.02 (d, J=177.1 Hz), 63.26, 56.29 (d, J=19.0 Hz), 55.39, 52.73, 50.02 (d, J=18.5 Hz), 46.43, 46.11, 32.94, 27.04 (d, J=1.7 Hz), 21.11. ¹⁹F NMR (376 MHz, Methanol-d₄) δ−201.62.

LC-MS (ESI) [M+H]⁺=481.23

### N-[(3S,4S)-3-fluoro-1-[(4-methoxyphenyl)methyl]piperidin-4-yl]-3-[6-(4-methylpiperazin-1-yl)-[1,2,4]triazolo[4,3-b]pyridazin-3-yl]propanamide 29b (LIT-TB052)

General procedure D for the synthesis of 28a was followed using tert-butyl (3S,4S)-3-fluoro-4-{3-[6-(4-methylpiperazin-1-yl)-[1,2,4]triazolo[4,3-b]pyridazin-3-yl]propanamido}piperidine-1-carboxylate 27b (1 eq., 34 mg, 0.0693 mmol), 4-methoxybenzylchloride (1.1 eq., 12.2 mg, 10.5 μL, 0.0762 mmol) and K₂CO₃ (5 eq., 47.9 mg, 0.347 mmol) in DMF (0.7 ml). The crude was evaporated and purified by reverse phase chromatography (H₂O/MeOH), salified and lyophilized to yield 29b as a white solid (m=15.2 mg, yield=58%).

¹H NMR (400 MHz, Methanol-d₄) δ 7.88 (d, J=10.2 Hz, 1H), 7.34 (d, J=10.2 Hz, 1H), 7.24-7.19 (m, 2H), 6.90-6.85 (m, 2H), 4.33 (dtd, J=49.7, 9.4, 4.7 Hz, 1H), 3.84-3.73 (m, 1H), 3.78 (s, 3H), 3.66 (t, J=5.1 Hz, 4H), 3.55-3.47 (m, 2H), 3.37-3.33 (m, 2H), 3.11-3.06 (m, 1H), 2.80 (t, J=7.7 Hz, 2H), 2.80-2.74 (m, 1H), 2.59 (t, J=5.1 Hz, 4H), 2.36 (s, 3H), 2.12-2.05 (m, 2H), 1.89 (dtd, J=10.7, 5.4, 2.8 Hz, 1H), 1.45 (qd, J=12.0, 3.9 Hz, 1H). ¹³C NMR (101 MHz, Methanol-d₄) δ 173.89, 160.6, 156.8, 150.0, 144.0, 131.6, 130.4, 124.7, 116.6, 114.7, 90.7 (d, J=177.8 Hz), 62.6, 57.0 (d, J=24.9 Hz), 55.7, 55.4, 52.6 (d, J=18.4 Hz), 52.3, 46.4, 46.1, 33.3, 30.4 (d, J=7.0 Hz), 21.1. ¹⁹F NMR (376 MHz, Methanol-d₄) δ−189.7.

LC-MS (ESI) [M+H]⁺=511.27

### N-[(3R,4R)-3-fluoro-1-[(4-methoxyphenyl)methyl]piperidin-4-yl]-3-[6-(4-methylpiperazin-1-yl)-[1,2,4]triazolo[4,3-b]pyridazin-3-yl]propanamide 29c (LIT-TB053)

General procedure D for the synthesis of 28a was followed using tert-butyl (3R,4R)-3-fluoro-4-{3-[6-(4-methylpiperazin-1-yl)-[1,2,4]triazolo[4,3-b]pyridazin-3-yl]propanamido}piperidine-1-carboxylate 27c (1 eq., 48.3 mg, 0.0985 mmol), 4-methoxybenzylchloride (1.1 eq., 17.3 mg, 15 μL, 0.108 mmol) and K₂CO₃ (5 eq., 68 mg, 0.492 mmol) in DMF (0.7 ml). The crude was evaporated and purified by reverse phase chromatography (H₂O/MeOH), salified and lyophilized to yield 29c as a white solid (m=17.3 mg, yield=66%).

¹H NMR (400 MHz, Methanol-d₄) δ 7.88 (d, J=10.2 Hz, 1H), 7.34 (d, J=10.2 Hz, 1H), 7.24-7.19 (m, 2H), 6.90-6.85 (m, 2H), 4.33 (dtd, J=49.7, 9.4, 4.7 Hz, 1H), 3.84-3.74 (m, 1H), 3.79 (s, 3H), 3.66 (t, J=5.1 Hz, 4H), 3.55-3.47 (m, 2H), 3.37-3.33 (m, 2H), 3.12-3.06 (m, 1H), 2.80 (t, J=7.7 Hz, 2H), 2.80-2.74 (m, 1H), 2.59 (t, J=5.1 Hz, 4H), 2.36 (s, 3H), 2.12-2.05 (m, 2H), 1.89 (dtd, J=10.7, 5.4, 2.8 Hz, 1H), 1.46 (qd, J=12.0, 3.9 Hz, 1H). ¹³C NMR (101 MHz, Methanol-d₄) δ 173.9, 160.6, 156.8, 150.0, 144.0, 131.6, 130.4, 124.7, 116.6, 114.7, 90.7 (d, J=177.8 Hz), 62.6, 57.0 (d, J=25.0 Hz), 55.7, 55.4, 52.6 (d, J=18.4 Hz), 52.3, 46.4, 46.1, 33.3, 30.4 (d, J=6.9 Hz), 21.1. ¹⁹F NMR (376 MHz, Methanol-d₄) δ−189.7.

LC-MS (ESI) [M+H]⁺=511.25

Preparation of Triazolopyridines

Alternatively, the carbaisostere of compound 9a (LIT-TB001) has been prepared as reported in scheme 7. Starting from the known hydrazine-bromopyridine derivative 35, the reaction with the propanoic acid 4a, in presence of isobutyl chloroformiate, afforded the hydrazide 36 that was later cyclized under Mitsunobu conditions in presence of TMSN₃ into the triazolopyridine 37. The final compound 38 was obtained under Buchwald cross coupling reaction conditions.

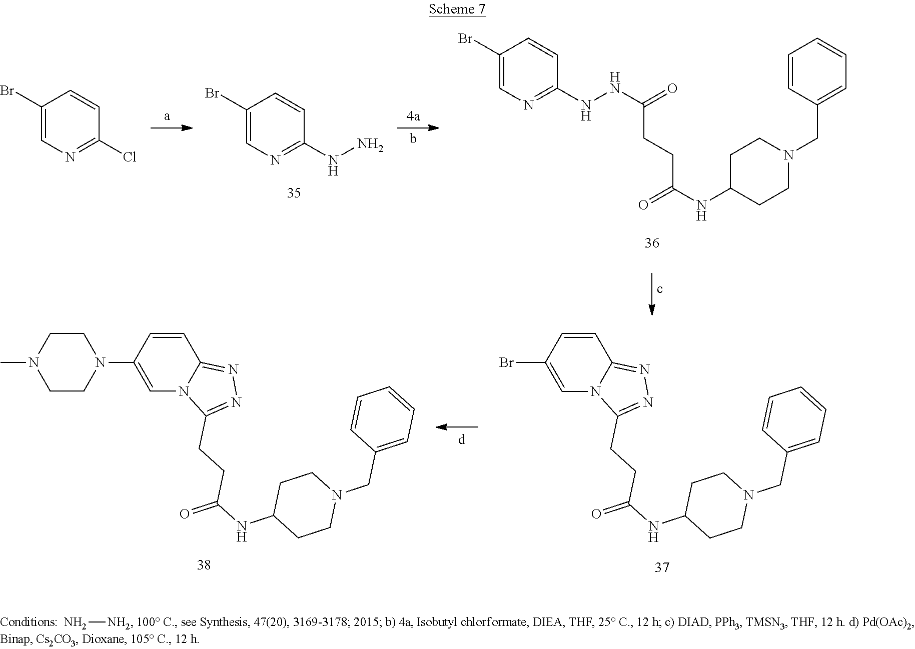

### Example 8: N-(1-benzylpiperidin-4-yl)-3-(6-(4-methylpiperazin-1-yl)-[1,2,4]triazolo[4,3-a]pyridin-3-yl)propanamide 38 (LIT-TB006)

### Step 1: N-(1-benzylpiperidin-4-yl)-4-(2-(5-bromopyridin-2-yl)hydrazinyl)-4-oxobutanamide 36

4-((1-benzylpiperidin-4-yl)amino)-4-oxobutanoic acid 4a (1.0 eq., 300 mg, 1.56 mmol) was suspended in THF (6 ml) followed by NMM (1.2 eq., 193.7 mg, 0.21 ml). Isobutyl chloroformate (0.5g, 0.49 mL) was then added dropwise to the solution and the resulting mixture was stirred 30 min at rt. 5-bromo-2-hydrazinylpyridine (1 eq., 300 mg, 1.59 mmol) was then added and the agitation was maintained for an additional hour. Volatiles were evaporated and the crude was dissolved in EtOAc (30 mL). The organic phase was washed once with 1 N Na₂CO₃ (15 mL), water (15 mL), brine (20 mL) and dried over Na₂SO₄, filtered and concentrated under reduce pressure. The residue was then purified by silica gel column chromatography using a gradient of 0% to 3% of NEt₃ in EtOAc: MeOH 9:1 to yield the title compound as a white solid (212 mg, 29%).

¹H NMR (400 MHz, CDCl₃) δ 5.62 (s, 1H), 8.11 (s, 1H), 7.50 (d, 1H, J=8.0 Hz), 7.29-7.20 (m, 5H); 6.96 (s, 1H), 6.54 (d, 1H, J=8.0 Hz), 5.93 (d, 1H, J=4.0 Hz), 3.73-3.65 (m, 1H), 3.45 (s, 2H), 2.76 (d, 2H, J=4.0 Hz), 2.49 (dd, 2H, J=8.0 Hz, J=4.0 Hz), 2.04 (t, 2H, J=12.0 Hz), 1.79 (d, 2H, J=12 Hz), 1.40 (dq, 2H, J=12 Hz, J=4.0 Hz). ¹³C NMR (101 MHz, CDCl₃) δ 172.5, 171.3, 158.1, 148.7, 140.5, 129.3, 128.4, 127.3, 110.9, 108.3, 63.1, 52.3, 46.9, 32.1, 31.4, 29.7

### Step 2: N-(1-benzylpiperidin-4-yl)-3-(6-bromo-[1,2,4]triazolo[4,3-a]pyridin-3-yl)propanamide 37

A solution of DIAD (109, 8g, 107.7 μL, 2.5 equiv.) and TMS-N₃ (62.56 mg, 0.54 mmol, 72.08 μl) in THF (0.4 mL) was slowly added to a solution of triphenylphosphine (142.4, 0.53 mmol, 2.5 equiv.), N-(1-benzylpiperidin-4-yl)-4-(2-(5-bromopyridin-2-yl)hydrazinyl)-4-oxobutanamide (100 mg, 0.21 mmol in THE (1.2 mL) and the resulting cloudy mixture was stirred at rt overnight. Silica gel was added to the mixture and the volatiles were evaporated. Flash chromatography of the crude product using a gradient of to 3% of Et₃N in EtOAc-MeOH 9:1 afforded the title compound as a pale yellow solid (m=53.2 mg, yield=55%).

¹H NMR (400 MHz, Methanol-d₄) δ 8.68 (s, 1H), 7.62 (d, 1H, J=8.0 Hz), 7.48 (d, 1H, J=8.0 Hz), 7.33-7.25 (m, 5H), 3.67-3.61 (m, 1H), 3.65 (s, 2H), 2.90 (d, 2H, J=12.0 Hz), 2.79 (t, 2H, J=8.0 Hz), 2.24 (t, 1H, J=12.0 Hz), 1.80 (m, 2H), 2.26 (dq, 2H, J=12.0 Hz, J=4.0 Hz). ¹³C NMR (101 MHz, Methanol-d₄) δ 173.1, 149.6, 148.3, 137.2, 133.1, 130.9, 129.4, 128.8, 125.3, 116.9, 109.8, 63.6, 53.0, 47.5, 33.6, 31.8, 21.1

### Step 3: N-(1-benzylpiperidin-4-yl)-3-(6-(4-methylpiperazin-1-yl)-[1,2,4]triazolo[4,3-a]pyridin-3-yl)propanamide 38 (LIT-TB006)

A microwave vial (oven-dried and under argon) was charged with N-(1-benzylpiperidin-4-yl)-3-(6-bromo-[1, 2, 4]triazolo[4,3-a]pyridin-3-yl)propanamide 37 (100 mg, 0.23 mmol, 1 equiv.), 1 methylpiperazine (22.64 mg, 25 μL, 0.23 mmol), Cs₂CO₃ (147.3 mg, 0.45 mmol, 2 equiv), Pd(OAc)₂ (1.02 mg, 2 mol %) and Binap (8.45 mg, 6 mol %) was added followed by dioxane (1.05 mL). The vial was properly capped and the mixture vessel was evacuated and backfilled with argon (process repeated 3 times) and heated at 105° C. overnight. After cooling to room temperature, silica gel was added and the resulting mixture was evaporated to dryness. Flash chromatography of the crude using EtOAc/MeOH/Et₃N 8:2:0.3 as eluent afforded the title compound (m=40 mg, yield=38%).

LC-MS (ESI) [M+H]⁺=462, 2979

Preparation of Imidazopyridines

The invention provides also a process for the preparation of imidazopyridine derivatives of general formula 44. Illustrative general synthetic method is given in scheme 8. A three component Michael-type (3CC) reaction involving bromo-imidazopyridine, Meldrum acid and formaldehyde led the corresponding 3-imidazo[1,2-a]pyridine-3-ylpropionic acid using a known procedure [18]. The reaction was conducted in the presence of a catalytic amount of L-proline, and afforded the corresponding “Michael-type” Yonemitsu adduct 41 which was first transformed to the stable ester 42 by ethanolysis and a copper-catalyzed concomitant decarboxylation and then converted to the corresponding amide 43 after successive alkaline hydrolysis and classical peptide coupling reaction. Finally a Buchwald type cross-coupling reaction led to the target compound 44 (LIT-TB013).

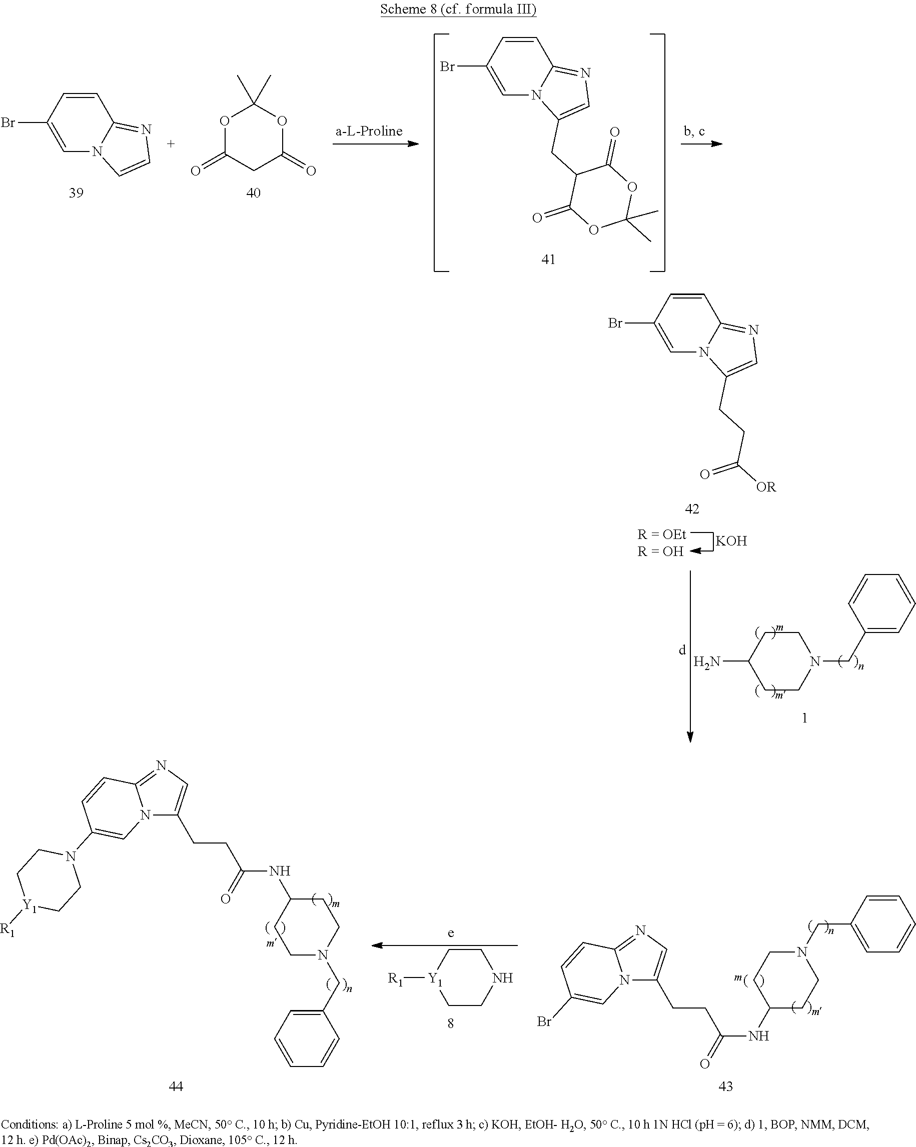

### Example 9: N-(1-benzylpiperidin-4-yl)-3-(6-(4-methylpiperazin-1-yl)imidazo[1,2-a]pyridin-3-yl)propanamide 44 (LIT-TB013)

### Step 1: ethyl 3-(6-bromoimidazo[1,2-a]pyridin-3-yl)propanoate 42

6-bromoimidazo[1,2-a]pyridine (1.50 g, 7.61 mmol, 1 equiv.), Meldrum acid (1 eq., 1.10 g, 7.61 mmol), paraformadehyde (1 eq., 228.6 mg, 7.61 mmol) and L-proline (43.8 mg, 5 mol %) were suspended in acetonitrile (29.23 mL) and the reaction mixture was stirred overnight at 50° C. under a nitrogen atmosphere. The precipitated product was collected by filtration and washed thoroughly with diethyl ether. The solid was dried (m=1.83 g, 5.18 mmol, yield=68%). The resulting compound 41 (1 eq., 1.50 g, 4.25 mmol) was dissolved in pyridine/EtOH(10:1 v/v, 5.5 mL), copper powder was added (12.75 mg, 0.20 mmol) and the mixture was refluxed for 3 h. The solvents were removed under reduced pressure. Flash chromatography of the crude using EtOAc as eluent afforded the title compound 42 (m=500 mg, yield=40%).

¹H NMR (400 MHz, CDCl₃) δ 8.04 (d, 1H, J=1.2 Hz), 7.43 (d, 1H, J=9.2 Hz), 7.36 (s, 1H), 7.15 (dd, 1H, J=9.2 Hz, J=1.2 Hz), 4.09 (q, 2H, J=7.2 Hz), 3.10 (t, 2H, J=15.2 Hz), 2.72 (t, 2H, J=14.8 Hz), 1.19 (t, 2H, J=7.2 Hz). ¹³C NMR (101 MHz, CDCl₃) δ 172.5, 151.6, 131.8, 126.9, 123.2, 123.1118.7, 112.6, 107.1, 60.9, 32.0, 19.4, 14.2.

### Step 2: N-(1-benzylpiperidin-4-yl)-3-(6-bromoimidazo[1,2-a]pyridin-3-yl)propanamide 43

Ethyl 3-(6-bromoimidazo[1,2-a]pyridin-3-yl) propanoate 42 (1 eq., 500 mg, 1.68 mmol) was dissolved in EtOH (10 mL) and then treated with potassium hydroxide (2 eq., 189 mg, 3.36 mmol, in 1 mL of H₂O) at 0° C. The resulting mixture was stirred at ambient temperature 1 hour. Volatiles were evaporated and the crude dissolved in H₂O (20 mL) and extracted with EtOAc (15 mL). The organic solvent was removed, and the remaining aqueous solution was acidified with 1 N HCl until the pH reached around 4. The generated solid was filtered and dried under reduced pressure to give the 3-(6-bromoimidazo[1,2-a]pyridin-3-yl)propanoic acid (m=340 mg, yield=75%)

The obtained acid (200 mg, 0.74 mmol, 1 equiv.), and BOP (349.5 mg, 0.74 mmol) were suspended in DCM (5.0 mL). NMM (112.8 mL, 122 μL, 1.11 mmol, 1.5 equiv.) was added and the reaction mixture was stirred at r.t. for 15 min1-benzylpiperidin-4-amine (141.5 mg, 0.74 mmol, 1 equiv.) was then added and the reaction was stirred at r.t. overnight (20 h). MeOH and silica were added and the crude was evaporated. The adsorbed compound on silica was then purified on silica gel chromatography (eluent MeOH/AcOEt 8/2) to yield the title compound 43 as a yellow (m=379 mg, yield=93%).

### Step 3: N-(1-benzylpiperidin-4-yl)-3-(6-(4-methylpiperazin-1-yl)imidazo[1,2-a]67yridazi-3-yl)propanamide 44 (LIT-TB013)

A microwave vial (oven-dried and under argon) was charged with N-(1-benzylpiperidin-4-yl)-3-(6-bromoimidazo[1,2-a]67yridazi-3-yl)propanamide (1 eq., 50 mg, 0.11 mmol), methylpiperazine (12.5 mg, 13.8 μL, 0.12 mmol), Cs₂CO₃ (2 eq., 73.8 mg, 0.23 mmol), Pd(OAc)₂ (0.8 mg, 3 mol %) and Binap (4.2 mg, 6 mol %) was added followed by dioxane (1.0 mL). The vial was properly capped and the mixture vessel was evacuated and backfilled with argon (process repeated 3 times) and heated at 105° C. overnight. After cooling to room temperature, silica gel was added and the resulting mixture was evaporated to dryness. A first Flash chromatography of the crude using EtOAc/MeOH/Et₃N 8:2:0.3 followed by a reverse C18 Flash chromatography (10 to 100% of MeOH in H₂O+0.05% HCl) afforded the title compound 44 (m=7 mg, yield=13%).

LC-MS [M+H]⁺=461.2

Preparation of Imidazopyridazines

The previous Michael-type (3CC) reaction using Meldrum acid and formaldehyde can be extended to imidazopyridazine derivatives (scheme 9). This reaction enabled the formation of the corresponding propionic acid 47 in presence of an electron donating group (OMe) on position 6 of the imidazopyridine moiety (cpd 46). Demethylation reaction was performed in presence of LiCl and p-toluenesulfonic acid, leading to the 6-chloroimidazo-pyridazine amide 49 after a chlorination reaction using POCl₃, followed by peptide coupling reaction with 1. The final compounds of formula 50 were last obtained by coupling 49 with various heterocyclic secondary amines 8 under basic conditions as previously described.

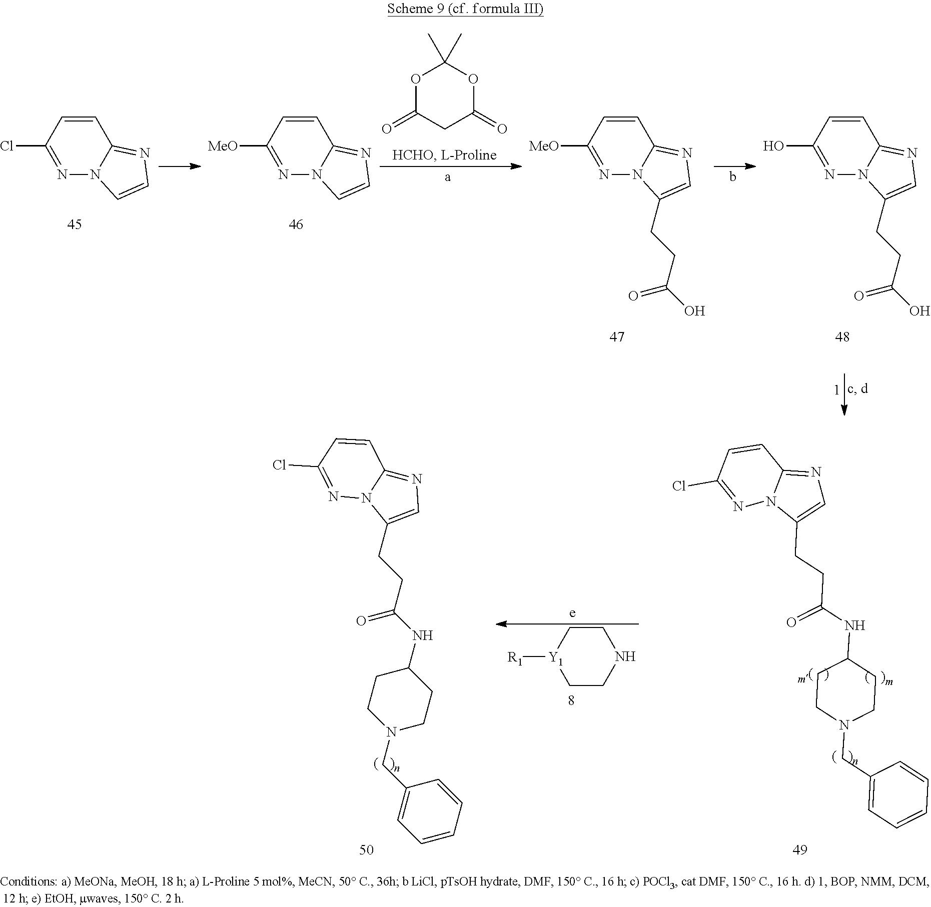

### Example 10: Preparation of N-(1-benzylpiperidin-4-yl)-3-(6-(4-methylpiperazin-1-yl)imidazo[1,2-b]pyridazin-3-yl)propanamide 50 LIT-TB014)

### Step 1: 6-Methoxyimidazo[1,2-b]pyridazine 46

Sodium methoxide (7.35 eq., 7.76g, 143.6 mmol) was added to a solution of 6-chloroimidazo[1,2-bb]pyridazine (3.0 g, 19.54 mmol) in anhydrous methanol (8 ml) at ambient temperature and the reaction mixture stirred for hours. The volatiles were removed by evaporation and the yellow oily residue was dissolved in dichloromethane (100 ml). The solution was washed with water (5×100 ml) until aqueous wash became neutral. The organic solution was dried (MgSO₄) and the solvent removed. The title compound (m=8.87 g, yield=91%) was obtained as a white solid.

¹H NMR (400 MHz, DMSO-d₆) δ 7.36 (d, J=9.3 Hz, 1H), 6.85 (d, J=9.3 Hz, 1H), 6.61 (s, 1H)¹³C NMR (101 MHz, CDCl₃) δ 160.2, 137.3, 132.4, 127.3, 116.8, 112.1, 54.4

### Step 2: 3-(6-methoxyimidazo[1,2-b]pyridazin-3-yl)propanoic acid 47

6-Methoxyimidazo[1,2-b]pyridazine (1 eq., 1.0 g, 6.7 mmol), Meldrum acid (1 eq., 0.97 g, 6.70 mmol), paraformaldehyde (1 eq., 201.3 mg, 6.70 mmol) and L-proline (38.6 mg, 5 mol %) were suspended in acetonitrile (30 mL) and the reaction mixture was stirred 36 h at 50° C. under a nitrogen atmosphere. The precipitated product was collected by filtration and washed thoroughly with diethyl ether and dried yielding the title compound as a white solid (m=1.0 g, yield=67%).

¹H NMR (400 MHz, DMSO-d₆) δ 12.71-12.01 (bs, 1H), 7.96 (d, J=9.6 Hz, 1H), 7.43 (s, 1H, J=9.6 Hz), 6.81 (d, J=9.6 Hz, 1H), 3.97 (s, 3H). ¹³C NMR (101 MHz, CDCl₃) δ 173.5, 159.5, 136.5, 129.9, 127.6, 127.5, 110.3, 54.3, 31.1, 18.8

### Step 3: 3-(6-hydroxyimidazo[1,2-b]pyridazin-3-yl)propanoic acid 48

The obtained acid (1 eq., 920 mg, 4.16 mmol) was suspended in DMF (11.5 mL). LiCl (5 eq., 881.6 mg, 20.8 mmol) was added followed by pTsOH hydrate (5 eq., 3.95 g, 20.79 mmol and the resulting mixture was heated overnight at 150° C. under nitrogen atmosphere. DMF was evaporated and the crude was suspended in water. The precipitated product was collected by filtration and washed thoroughly with diethyl ether and dried yielding the title compound 48 (m=600 mg, yield=70%).

¹H NMR (400 MHz, DMSO-d₆) δ 12.71-11.68 (bs, 1H), 7.96 (d, J=9.6 Hz, 1H), 7.43 (s, 1H, J=9.6 Hz), 6.83 (d, J=9.6 Hz, 1H), 3.10 (t, J=7.1 Hz, 2H), 2.73 (t, J=7.5 Hz, 2H).

LC-MS [M+H]⁺=208.0

### Step 4: N-(1-benzylpiperidin-4-yl)-3-(6-(4-methylpiperazin-1-yl)imidazo[1,2-b]pyridazin-3-yl)pro-panamide 50 (LIT TB014)

3-(6-hydroxyimidazo[1,2-b]pyridazin-3-yl)propanoic acid (1 eq., 200 mg, 0.96 mmol) and N(Me)₄Cl (1 eq. 105.8 mg, 0.96 mmol) were suspended in POCl₃ (1.1 mL) and the resulting mixture was heated overnight under a nitrogen atmosphere. After cooling at rt, DMF was evaporated and the crude was purified by flash chromatography using EtOAc/MeOH/AcOH (8:2:0.5) as eluent to yield the 3-{6-chloroimidazo[1,2-b] pyridazine-3-yl}propanoic acid (100 mg, 46%). LC-MS (ES+APCl): 282.2 [M+Na⁺], 208.0 [M+H]⁺ The above product (1 eq., 50 mg, 0.22 mmol) BOP (1.2 eq., 117.6 mg, 0.22 mmol) and NMM (1.5 eq., 33.6 mg, 0.33 mmol) were suspended in DCM (1.5 mL) and, and the reaction mixture was stirred at r.t. for 15 min. 4-amino-1-benzylpiperidine (42.17 mg, 45.3 μL, 0.22 mmol) was then added and the reaction was stirred at r.t. overnight (20 h). Water was then added (15 mL) to the resulting mixture, and the aqueous solution was extracted twice with DCM (3×8 mL). The organic phases were combined, dried over Na₂SO₄, filtered and concentrated under reduce pressure. The resulting oil was purified by silica gel flash chromatography using EtOAc/MeOH 8/2 as eluent to yield N-(1-benzylpiperidin-4-yl)-3-{6-chloroimidazo[1,2-b]pyridin-3-yl}propanamide 49 (65 mg, 74%). LC-MS [M+H]⁺=398.2 Using the same procedure A as described for 9a (LIT-TB001) and starting from the above product 49 (1 eq. 40 mg, 0.10 mmol) and 1-methylpiperazine (20.14 mg, 22.3 μL, 0.20 mmol, 2 equiv.) the title compound was obtained in 65% yield.

LC-MS (ES+APCl): 484.2 [M+Na⁺], 462.2 [M+H⁺].

Preparation of Triazolopyridazines

The invention provides also a process for the preparation of appropriate N-substituted-triazolo[4,3-b]pyridazin-3-yl)propylpiperidin-4-amine of formula (Scheme 10). Starting from N-benzyl-piperidin-4-one 51 an amination reaction with methyl 4-aminobutyrate in presence of NaBH₃CN, afforded the N-benzyl piperidin-4-amino-ethylbutanoate 52. In order to avoid intramolecular cyclisation, 53 was first N-Boc protected (cpd 53) and then submitted after saponification, to a peptide-type coupling reaction with hydrazino pyridazine 5 under conditions well-known in the art. Cyclisation under strong acidic conditions (135° C.) followed by an SNAr-type amination reaction in presence of 8a-g led to the target products 56.

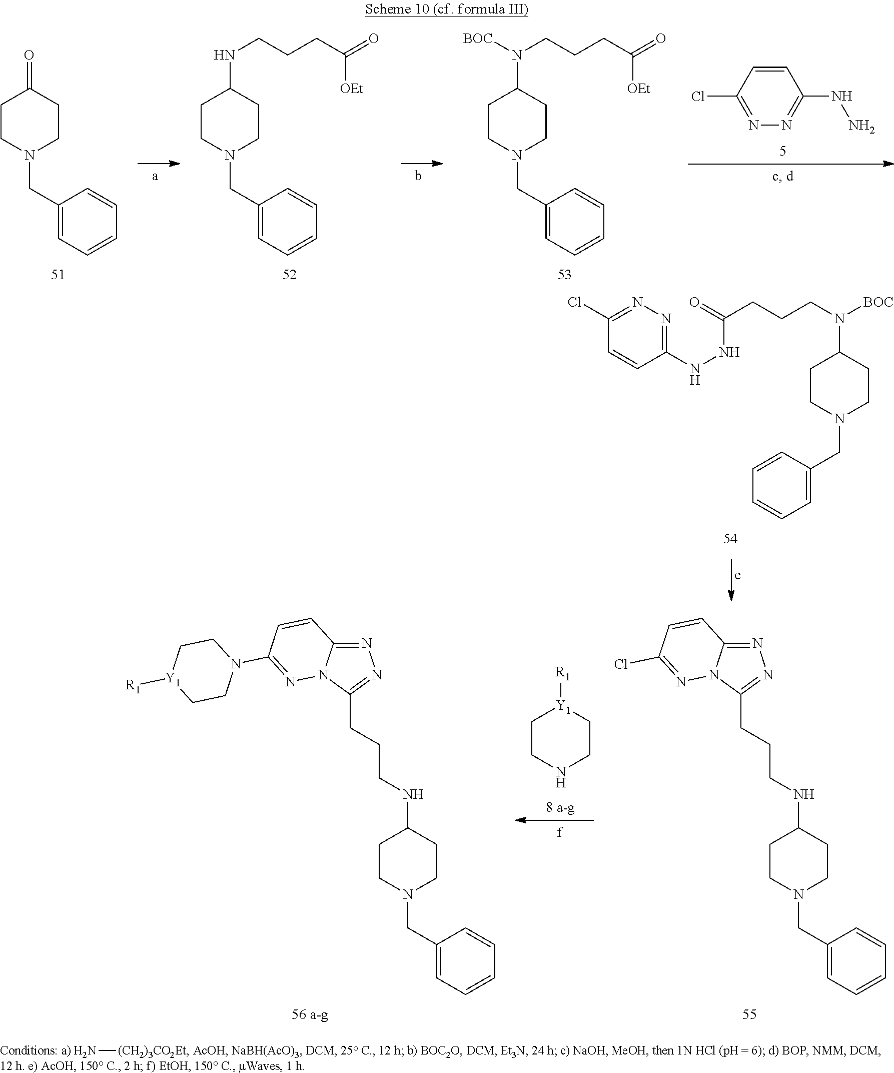

### Example 11: Preparation of 1-benzyl-N-(3-(6-(4-methylpiperazin-1-yl)-[1,2,4]triazolo[4,3-b]pyridazin-3-yl)propyl)piperidin-4-amine 56a (LIT-TB015)

### Step 1: 4-((1-benzylpiperidin-4-yl)amino)butanoate 52

To an ice cold solution of 1-benzyl-piperidin-4-one 51 (1 eq., 1.00 g, 5.28 mmol) in CH₂Cl₂ (35 ml) was added methyl 4-aminobutyrate hydrochloride (1 eq., 0.88 g, 5.28 mmol), acetic acid (3.5 eq., 1.1 ml, 18.49 mmol), Et₃N (1.5 eq., 802 mg, 1.1 mL, 3 mmol) and sodium triacetoxyborohydride (3 eq., 3.5g, 3 mmol). The mixture was allowed to reach room temperature and was stirred for 16h. After that time the solution was washed with saturated potassium hydrogen carbonate solution, dried (Na₂SO₄) and concentrated. The crude was purified by flash chromatography using EtOAc-MeOH (8:2) to yield ethyl 4-((1-benzylpiperidin-4-yl)amino)butanoate 52 (m=1.15 g, yield=71%).

¹H NMR (400 MHz, CDCl₃) δ 7.25-7.21 (m, 4H), 7.20-7.14 (m, 1H), 4.05 (q, 2H, J=7.0 Hz). 343 (s, 2H), 2.82-2.75 (m, 2H), 2.62-2.61 (bs, 1H), 2.59 (t, 2H, J=7.2 Hz), 2.43-2.36 (m, 1H), 2.28 (t, 2H, J=7.2 Hz), 1.93-1.63 (m, 4H), 1.33 (dq, 2H, J=11.8 Hz, J=3.6 Hz). ¹³C NMR (101 MHz, CDCl₃) δ 174.6, 138.3, 129.2, 128.3, 127.0, 62.9, 52.7, 48.7, 42.7, 31.4, 29.0, 18.1

### Step 2: ethyl 4-((1-benzylpiperidin-4-yl)(tert-butoxycarbonyl)amino)butanoate 53

To a stirred solution of ethyl 4-((1-benzylpiperidin-4-yl)amino)butanoate (1 eq., 1.2 g, 3.94 mmol) in DCM (15 mL) was added Et₃N (2 eq., 797.7 mg, 7.88 mmol,) followed by Boc₂O (1.5 eq., 1.29 g, 1.26 mmol) and the resulting mixture was stirred overnight. After that time the solution was washed with water, dried (Na₂SO₄) and concentrated. The crude was purified by flash chromatography to yield the title compound 53 (m=1.35 g, yield=85%).

¹H NMR (400 MHz, CDCl₃) δ 7.28-7.11 (m, 5H), 4.06 (q, 2H, J=7.2 Hz), 3.96-3.79 (m, 1H), 3.41 (s, 2H), 3.11-2.99 (m, 2H), 2.98 (d, 2H, J=12.0 Hz), 2.20 (t, 2H, J=7.7 Hz), 2.02-1.91 (m, 2H), 1.79-1.70 (m, 2H), 1.68-1.63 (m, 4H), 1.39 (s, 8H), 1.19 (t, 3H, J=7.2 Hz)¹³C NMR (101 MHz, CDCl₃) δ 173.2, 155.6, 129.1, 128.2, 127.0, 79.5, 63.0, 60.3, 53.3, 42.2, 31.9, 30.1, 25.8, 14.3.

### Step 3: tert-butyl (1-benzylpiperidin-4-yl)(4-(2-(6-chloropyridazin-3-yl)hydrazinyl)-4-oxobutyl)carbamate 54

Ethyl 4-((1-benzylpiperidin-4-yl)(tert-butoxycarbonyl)amino)butanoate 53 (1 eq., 1.3 g, 3.21 mmol) was diluted in MeOH (5 mL). 1N NaOH (15 mL) was added and the reaction mixture was stirred at r.t. overnight. The crude was acidified to pH=6 with 2N HCl, evaporated. The crude product (1 eq., 1.0 g, 2.66 mmol), BOP (1.2 eq., 1.4 g, 2.66 mmol,) and NMM (2.5 eq., 0.67 g, μl, 6.64 mmol) were suspended in DCM (15 mL) and, and the reaction mixture was stirred at r.t. for 15 min. 3-chloro-6-hydrazinopyridazine 5 (1 eq., 384 mg, 2.66 mmol,) was then added and the reaction was stirred at r.t. overnight (20 h). After evaporation of the volatiles, the crude was directly purified by silica gel flash chromatography using EtOAc/MeOH/Et₃N 8/2/0.3 as eluent to yield the title compound (m=1.0 g, yield=75%).

¹H NMR (400 MHz, CDCl₃) δ 8.50 (bs, 1H), 7.52 (bs, 1H), 7.42-729 (m, 5H), 7.27 (d, 1H, J=9.5 Hz), 7.04 (d, 1H, J=9.9 Hz), 4.30-4.13 (m, 2H), 4.04-3.89 (m, 1H), 3.7 (t, 2H, J=4.9 Hz), 3.45 (bs, 2H), 3.15-3.04 (m, 2H), 2.83 (t, 2H, J=12.1 Hz), 2.27 (t, 2H, J=7.2 Hz), 1.85-1.77 (m, 4H), 1.36 (s, 9H).

LC-MS (ES+APCl): 501 (M-H⁺), 401 (-Boc)

### Step 4: 1-benzyl-N-(3-(6-(4-methylpiperazin-1-yl)-[1,2,4]triazolo[4,3-b]pyridazin-3-yl)propyl)piperidin-4-amine 56a (LIT-TB015)

A microwave vial was charged with ethyl 4-((1-benzylpiperidin-4-yl)(tert-butoxycarbonyl) amino) butanoate 54 (1 eq., 400 mg, 0.82 mmol) and acetic acid (1.87 mL). The vial was properly capped and the mixture vessel was heated at 110° C. for 2 h. The mixture was cooled to r.t. and evaporated. The crude was co-evaporated with cyclohexane and was triturated with cold ether. The white solid was collected by filtration (210 mg, LC/MS 385.2 [M+H]) to yield compound 55 that was used in the next step without further purification.

Using the same procedure A described for 9a (LIT-TB001) and starting from compound 55 (1 eq., 100 mg, 0.25 mmol.) and 1-methylpiperazine 8a (2 eq., 100.1 mg, 57.6 μl) the title compound 56a was obtained under microwaves irradiation (m=40 mg, yield=34%).

LC-MS [M+H]⁺=449.2; 471.2 (M+Na)

Preparation of 57 (LIT-TB-058)

The invention provides also a process for reductive dehalogenation of 6-chlorotriazolopyridazine derivatives. In particular, 7a-f were used as substrates in halogen/metal exchange in the presence of Pd(PPh₃)₄ and HCOOH as reducing agent (see scheme 11).

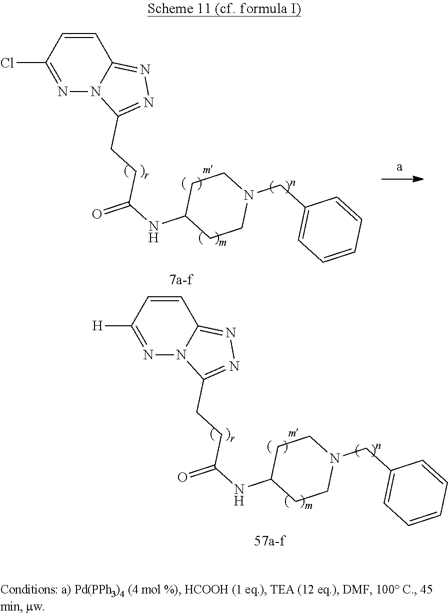

### Example 12: Preparation of 3-([1,2,4]triazolo[4,3-b]pyridazin-3-yl)-N-(1-benzylpiperidin-4-yl)propanamide 57a (LIT-TB058)

To a solution of N-(1-benzylpiperidin-4-yl)-3-{6-chloro-[1,2,4]triazolo[4,3-b]pyridazin-3-yl}propane amide 7a (1 eq., 100 mg, 0.25 mmol) in in dry DMF (2 mL) were added TEA (12 eq., 314.6 mg, 0.43 mL, 3.1 mmol), Pd(PPh₃)₄ (4 mol %, 11.6 mg). The vial was capped properly and degassed, and the contents were stirred at room temperature for 10 min. Then a solution of formic acid (1 eq., 11.54 mg, 9.5 μl, 1 mmol) in dry DMF (0.4 mL) was added and the reaction mixture was heated by microwave irradiation at 100° C. for 45 min. After it was cooled, the reaction mixture was concentrated and purified by silica gel flash chromatography using DCM/MeOH, 90/10+2% NH₃ to give the title compound after salification as yellow solid (m=26 mg, yield=26%).

¹H NMR (400 MHz, Methanol-d₄) δ 8.58 (dd, J=4.2 Hz, J=1.6 Hz, 1H), 8.2 (dd, J=9.5 Hz, J=1.6 Hz, 1H), 7.36 (dd, J=9.5 Hz, J=4.3 Hz), 7.35-7.31 (m, 4H), 7.31-7.25 (m, 1H), 3.71-3.60 (m, 1H), 3.52 (s, 2H), 3.49 (t, J=7.5 Hz, 2H), 2.92-2.80 (m, 2H), 2.84 (t, J=7.5 Hz, 2H), 2.13 (dt, J=11.6 Hz, J=2.0 Hz, 2H), 1.82 (dd, J=13.1 Hz, J=3.5 Hz), 1.5 (dq, J=11.9 Hz, J=3.5 Hz, 2H).). ¹³C NMR (101 MHz, Methanol-d₄) δ 171.7, 149.5, 146.0, 144.4, 137.1, 129.4, 127.9, 127.0, 123.9, 121.1, 62.6, 51.9, 46.5, 31.6, 30.9, 19.7.

LC-MS [M+H]⁺=365.20

Preparation of Analogues 60a-f

The invention provides also a process for the direct introduction at position of a 4-Methyl tetrahydropyridine moiety with the mean of N-methyl-piperid-3-en-4-yl boronate 58 under Suzuki-Miyaura conditions, followed by hydrogenation over Pd/C (Scheme 12).

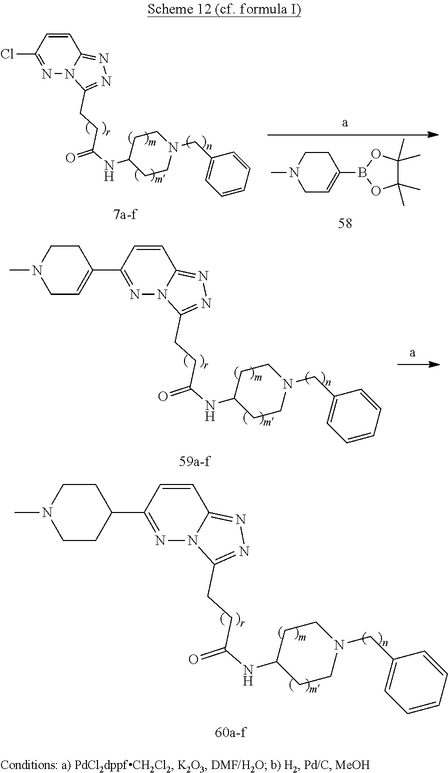

### Example 13: Preparation of N-(1-benzylpiperidin-4-yl)-3-(6-(1-methylpiperidin-4-yl)-[1,2,4]triazolo [4,3-b]pyridazin-3-yl)propanamide 60a (LIT-TB059)

N-(1-benzyl-4-piperidyl)-3-(6-chloro-[1,2,4]triazolo[4,3-b]pyridazin-3-yl)propanamide 7a (200 mg, 0.50 mmol, 1.0 eq.) was solubilized in dimethylformamide (10 mL). After addition of boronic acid pinacol ester 58 (110 mg, 0.50 mmol, 1.0 eq.), potassium carbonate (210 mg, 1.50 mmol, 3.0 eq.) and 2 drops of water, reaction mixture was degassed by argon bubbling for 20 minutes. Palladium complex PdCl₂dppf.CH₂Cl₂ (41 mg, 0.05 mmol, 0.1 eq.) was added portionwise and the reaction vessel was sealed and heated at 80° C. for 18h. After cooling down, solvent were removed under vacuum and the residue was purified by flash chromatography [Biotage®; column Biotage® 24g; eluant: EtOAc/MeOH; gradient: 100/0→100/0 (2 CV), 100/0-70/30 (12CV) then 70/30→70/30 (3CV)] affording compound (120 mg, 52% yield) as a dark red powder. Confirmed by LCMS: m/z=460.2 (M+H).

N-(1-benzyl-4-piperidyl)-3-[6-(1-methyl-3,6-dihydro-2H-pyridin-4-yl)-[1,2,4]triazolo [4,3-b]pyridazin-3-yl]propenamide 59 (120 mg, 0.26 mmol, 1.0 eq.) was solubilized in methanol (30 mL). After addition of palladium 10% on activated carbon (145 mg, 0.14 mmol, 0.5 eq.), reaction mixture was hydogenated under dihydrogen pressure (4 bars) at 20° C. for 6h. Reaction mixture was filtered through Celite® pad and solvents were evaporated under vacuum. The residue was purified by flash chromatography [Biotage®; column AIT® 4g; eluant: DCM/MeOH; gradient: 90/100→80/20 (10 CV)] affording compound 8 (53 mg, 44% yield) as a beige powder. Further lyophilization was proceeded in order to remove solvents traces.

¹H NMR (300 MHz, CDCl₃) δ 7.98 (d, J=9.6 Hz, 1H), 7.33-7.22 (m, 5H), 7.02 (d, J=9.6 Hz, 1H), 6.08 (d, J=7.7 Hz, 1H), 3.82-3.72 (m, 1H), 3.49-3.43 (m, 4H), 3.04-2.99 (m, 2H), 2.89 (t, J=7.1 Hz, 2H), 2.79-2.74 (m, 3H), 2.35 (s, 3H), 2.17-2.06 (m, 4H), 1.98-1.93 (m, 4H), 1.93-1.83 (m, 2H), 1.53-1.39 (m, 2H). ¹³C NMR (75 MHz, CDCl₃) δ 170.7, 160.2, 149.3, 143.8, 138.3, 129.1 (2C), 128.2 (2C), 127.0, 124.7, 119.9, 63.0, 55.3 (2C), 52.2 (2C), 46.6, 46.3, 41.7, 32.5, 32.0 (2C), 30.8 (2C), 20.3 LCMS: m/z=462.2 (M+H).

Preparation of Pyrazolopyridines

Alternatively, the triazolopyridazine ring can be replaced by a pyrazolopyridine ring of general structure 66, in a 4-step sequence, as depicted in the following scheme 13.

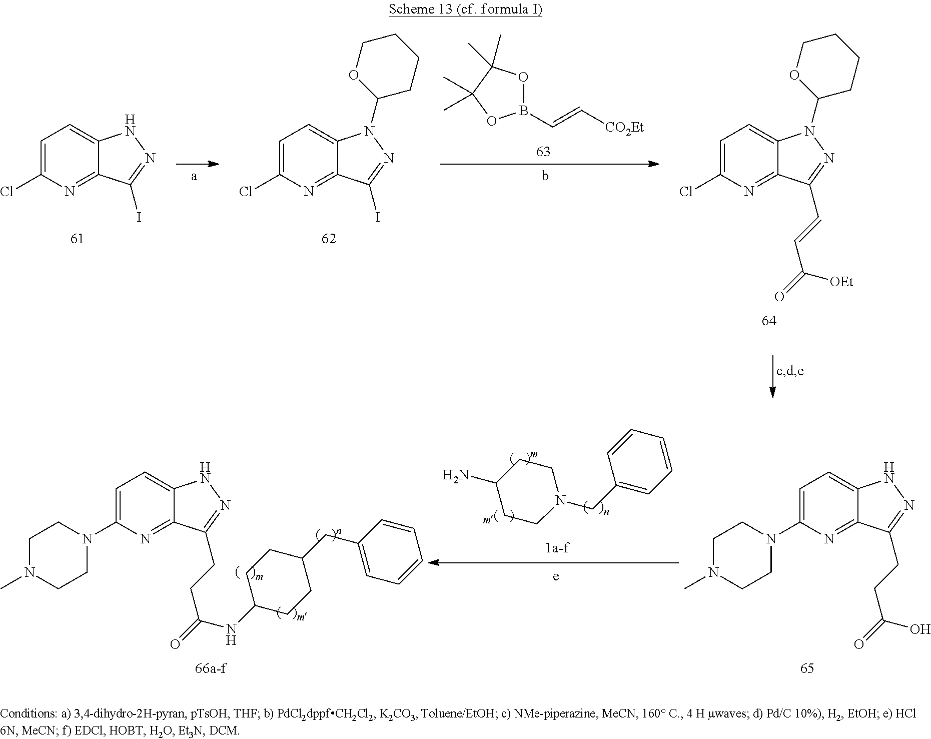

### Example 14: Preparation of N-(1-benzylpiperidin-4-yl)-3-(5-(4-methylpiperazin-1-yl)-1H-pyrazolo[4,3-b]pyridin-3-yl)propanamide 66a (LIT-TB060)

### Step 1: 5-chloro-3-iodo-1-(tetrahydro-2H-pyran-2-yl)-1H-pyrazolo[4,3-b]pyridine 62

5-Chloro-3-iodo-1H-pyrazolo[4,3-b]pyridine 1 (1.0 g, 3.60 mmol, 1.0 eq.), 3,4-dihydro-2H-pyran (650 mg, 7.70 mmol, 0.7 mL, 2.1 eq.) and para-toluene sulfonic acid (150 mg, 0.80 mmol, 0.2 eq.) were solubilized in THE (10 mL) and stirred at 60° C. for 18h. After cooling down to RT, NaHCO₃ saturated solution (50 mL) was added and the mixture was extracted with ethyl acetate (3×75 mL). Organic layers were dried over magnesium sulfate and evaporated under vacuum. The residue was purified by flash chromatography [Biotage®; column AIT® 80g; eluent: Cyclohexane/DCM; gradient: 100/0→100/0 (3 CV), 100/0→0/100 (20CV)] affording compound (1.30 g, 99% yield) as a colorless gum.

### Step 2: ethyl (E)-3-(5-chloro-1-(tetrahydro-2H-pyran-2-yl)-1H-pyrazolo[4,3-b]pyridin-3-yl)acrylate 64

5-Chloro-3-iodo-1-tetrahydropyran-2-yl-pyrazolo[4,3-b]pyridine 62 (1.0 g, 2.75 mmol, 1.0 eq.) was solubilized in a mixture of toluene (10 mL) and ethanol (5 mL). After addition of boronic acid pinacol ester 63 (810 mg, 3.58 mmol, 1.3 eq.), and potassium carbonate (2M) aqueous solution (5.60 mmol, 2.8 mL, 2.0 eq.), reaction mixture was degassed by argon bubbling for 20 minutes. Palladium complex (115 mg, 0.14 mmol, 0.05 eq.) was added portionwise and the reaction vessel was sealed and heated at 110° C. for 18h. After cooling down to RT, water (20 mL) was added and the mixture was extracted with ethyl acetate (3×50 mL). Organic layers were dried over magnesium sulfate and evaporated under vacuum. The residue was purified by flash chromatography [Biotage®; column AIT® 80g; eluant: Cyclohexane/EtOAc; gradient: 90/10→60/40 (20 CV)] affording compound (m=475 mg, yield=51%) as a white solid. Confirmed by LCMS: m/z=336.3 (M+H).

### Step 3: 3-(5-(4-methylpiperazin-1-yl)-1H-pyrazolo[4,3-b]pyridin-3-yl)propanoic acid 65

Ethyl (E)-3-(5-chloro-1-tetrahydropyran-2-yl-pyrazolo[4,3-b]pyridin-3-yl)prop-2-enoate 64 (470 mg, 1.40 mmol, 1.0 eq.) was solubilized in a mixture of N-methylpiperazine 6 (5 mL) and MeCN (5 mL). Reaction mixture was heated at 160° C. for 4h under micro-wave irradiations. Solvents were evaporated under vacuum and the residue was purified by flash chromatography [Biotage®; column Biotage® 24g; eluent: DCM/MeOH; gradient: 90/10→80/20 (20 CV)] affording compound 7 (340 mg, 60% yield) as a brown oil. Confirmed by LCMS: m/z=400.50 (M+H).

Ethyl (E)-3-[5-(4-methylpiperazin-1-yl)-1-tetrahydropyran-2-yl-pyrazolo[4,3-b]pyridin-3-yl]prop-2-enoate (330 mg, 0.83 mmol, 1.0 eq.) was solubilized in ethanol (30 mL). After addition of palladium 10% on activated carbon (100 mg, 0.09 mmol, 0.1 eq.), reaction mixture was hydrogenated under hydrogen pressure (4 bars) at 50° C. for 24h. Reaction mixture was filtered through Celite @ pad and solvents were evaporated under vacuum affording 3-[5-(4-Methylpiperazin-1-yl)-1H-pyrazolo[4,3-b]pyridin-3-yl]propanoic acid (m=335 mg, yield=99%) as a brown oil. Confirmed by LCMS: m/z=402.1 (M+H).

3-[5-(4-Methylpiperazin-1-yl)-1H-pyrazolo[4,3-b]pyridin-3-yl]propanoic acid (330 mg, 0.83 mmol, 1.0 eq.) was solubilized in acetonitrile (5 mL). After addition of HCl (6N) aqueous solution (5.0 mL), reaction mixture was heated at 100° C. for 30 minutes under micro-wave irradiations. Solvents were evaporated under vacuum, and the aqueous residue was washed with dichloromethane (3×20 mL). Aqueous layer was evaporated and dried under vacuum, affording compound 65 in a complex mixture with salts. The residue was used in the following step without any further purification. Confirmed by MS: m/z=290.25 (M+H).

### Step 4: N-(1-benzylpiperidin-4-yl)-3-(5-(4-methylpiperazin-1-yl)-1H-pyrazolo[4,3-b]pyridin-3-yl) propanamide 66a

Crude 3-[5-(4-methylpiperazin-1-yl)-1H-pyrazolo[4,3-b]pyridin-3-yl]propanoic acid 65 (crude, 0.83 mmol theorical, 1.0 eq.) and 1-benzylpiperidin-4-amine 10 (280 mg, 1.47 mmol, 0.30 mL, 1.8 eq.) were solubilized in dimethylformamide (10 mL). EDCl.HCl (315 mg, 1.66 mmol, 2.0 eq.), HOBt (225 mg, 1.66 mmol, 2.0 eq.) and Et₃N (725 mg, 7.17 mmol, 1.0 mL, 8.6 eq.) were added to reaction mixture which was stirred at room temperature for 24h. Reaction mixture was filtered and the filtrate was evaporated to dryness under high vacuum. Water (10 mL) was added to the residue. The aqueous residual solution was washed successively with ethyl acetate (3×20 mL) and with dichloromethane (3×20 mL). Aqueous layer was evaporated and dried under vacuum. The residue was solubilized in isopropanol and precipited by diisopropyl ether. After trituration and filtration, the filtrate was evaporated under vacuum. Another trituration in dichloromethane followed by a filtration led to detection of the targeted compound 11 in the filtrate. The residue containing 11 was purified by flash chromatography [Biotage®; column Biotage® 24g; eluent: EtOAc/MeOH; gradient: 100/0→100/0 (3 CV), 100/0→70/30 (15CV), then 70/30→70/30 (15CV) followed by DCM/NH₃ (7N) in MeOH; gradient: 100/0→100/0 (3 CV), 100/0 □ 70/30 (15CV) then 70/30→70/30 (5CV)] to afford compound in mixture with EDCl derivative. A second purification by semi-preparative HPLC (Gilson PLC 2020, column C8 Princeton SPHER.60-10 μm, gradient: water/acetonitrile (0.1% HCOOH) 95/5→95/5, 10 minutes and 95/5→0/100, 25 minutes) was done, followed by a direct lyophilization, affording pure compound 66 (22 mg, 7% yield) as a beige powder (formiate salt 0.3 eq.). The hydrochloride form of 66 was prepared by solubilization in dioxane (5.0 mL) and addition of HCl (4N) solution in dioxane (5.0 mL). After 1h of stirring at RT, solvents were evaporated and the residue was lyophilized to afford 66a as a hydrochloride salt (m=22 mg, yield=5%) as a beige powder.

¹H NMR (300 MHz, DMSO-d6): δ 7.78 (d, J=7.6 Hz, 1H), 7.71 (d, J=9.2 Hz, 1H), 7.33-7.22 (m, 5H), 7.02 (d, J=9.2 Hz, 1H), 3.65-3.30 (m, 7H), 3.05-2.98 (m, 2H), 2.80-2.72 (m, 2H), 2.65-2.50 (m, 5H), 2.31 (s, 3H), 2.11-2.25 (m, 2H), 1.70-1.65 (m, 2H), 1.43-1.35 (m, 2H). ¹³C NMR (75 MHz, DMSO-d6): δ 170.8, 163.3, 155.4, 137.5, 136.4, 129.3, 129.0, 128.2, 127.1, 120.6, 109.4, 61.7, 54.0, 51.7, 45.5, 45.3, 45.0, 34.1, 31.1, 21.7.

LCMS: m/z=462.2 (M+H).

Synthesis of a Fluorescent Analogue (LIT-TB043)

A fluorescent analogue of compound 9a (LIT-TB001) can be prepared by coupling a fluorogenic probe (e.g. DY-647P1-NHS-Ester) to a properly substituted primary amine as indicated in Scheme 14.

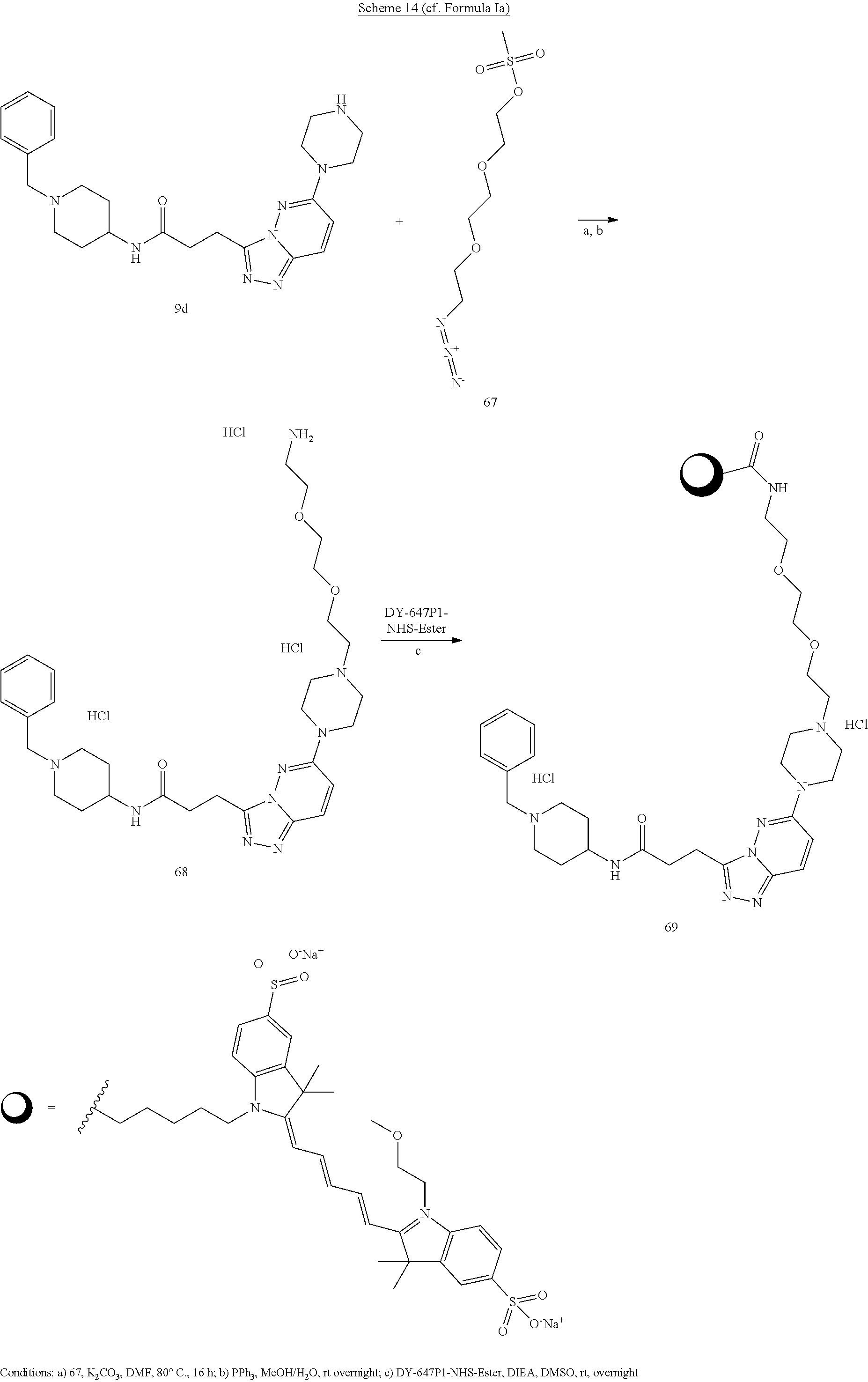

### Preparation of (2E)-1-[6-[2-[2-[2-[4-[3-[3-[(1-benzyl-4-piperidyl)amino]-3-oxo-propyl]-[1,2,4]triazolo[4,3-b]pyridazin-6-yl]piperazin-1-yl]ethoxy]ethoxy]ethylamino]-6-oxo-hexyl]-2-[(2E,4E)-5-[1-(2-methoxyethyl)-3,3-dimethyl-5-sulfonato-indol-1-ium-2-yl]penta-2,4-dienylidene]-3,3-dimethyl-indoline-5-sulfonate;dihydrochloride (LIT-TB043)

### Step 1: 3-(6-(4-(2-(2-(2-aminoethoxy)ethoxy)ethyl)piperazin-1-yl)-[1,2,4]triazolo[4,3-b]pyridazin-3-yl)-N-(1-benzylpiperidin-4-yl)propanamide hydrochloride 68

N-(1-benzylpiperidin-4-yl)-3-[6-(piperazin-1-yl)-[1,2,4]triazolo[4,3-b]pyridazin-3-yl]propanamide 9d (1 eq., 10.4 mg, 0.0232 mmol), 2-[2-(2-azidoethoxy)ethoxy]ethyl methanesulfonate 67 (1.5 eq., 8.81 mg, 0.0348 mmol) and K₂CO₃ (2 eq., 6.41 mg, 0.0464 mmol) were solubilized in dry DMF (0.2 ml). The reaction was flushed thrice with Argon and the mixture was stirred at 80° C. for 16 h. The crude was filtered over a pad of celite and washed with MeOH. The filtrate was evaporated to yield a yellowish solid (compound 69) which was solubilized in a mixture MeOH/H₂O (3/1, 1 ml). PPh₃ (2.5 eq., 15.2 mg, 0.058 mmol) was added and the reaction was stirred at r.t. overnight. DMSO was added to the crude and then the mixture was evaporated. The remaining DMSO phase was purified by reverse phase chromatography (H₂O+0.05% HCl/MeOH) to yield the compound as white solid (m=7.0 mg, yield=44%).

### Step 2: (2E)-1-[6-[2-[2-[2-[4-[3-[3-[(1-benzyl-4-piperidyl)amino]-3-oxo-propyl]-[1,2,4]triazolo[4,3-b]pyridazin-6-yl]piperazin-1-yl]ethoxy]ethoxy]ethylamino]-6-oxo-hexyl]-2-[(2E,4E)-5-[1-(2-methoxyethyl)-3,3-dimethyl-5-sulfonato-indol-1-ium-2-yl]penta-2,4-dienylidene]-3,3-dimethyl-indoline-5-sulfonate;dihydrochloride 69 (LIT-TB043)

3-[6-(4-{2-[2-(2-Aminoethoxy)ethoxy]ethyl}piperazin-1-yl)-[1,2,4]triazolo[4,3-b]pyridazin-3-yl]-N-(1-benzylpiperidin-4-yl)propanamide hydrochloride 68 (1 eq., 0.855 mg, 0.00124 mmol) and DY-647P1-NHS-Ester (1 eq., 1 mg, 0.00124 mmol) were solubilized in dry DMSO (0.3 ml). DIEA (5 eq., 0.802 mg, 1.03 μL, 0.0062 mmol) was added and the reaction was flushed thrice with Ar. The reaction was stirred overnight at r.t. The crude was directly purified by reverse phase chromatography (H₂O+0.05% HCl/MeOH) to yield LIT-TB043 as a blue solid (m=1.58 mg, yield=98%).

LC-MS [2 Na (m/2)]=646

### II. Results

Material

Recombinant human BDNF and NGF were obtained from Peprotech. Recombinant human TrkB^{ECD}-Fc was obtained from R&D Systems and BDNF-biotin was purchased from Alomone Labs. AAV-GCAMP6F viruses were produced at U Penn vector Core. Phosphatase inhibitor cocktail2 was purchased from Roche and protease inhibitor Complete ultra-cocktail was purchased from Sigma. Antibodies were obtained from different sources, as follows: polyclonal anti-TrkB, anti-phosphotyrosine (4G10) and anti-pY816-TrkB were from Millipore, monoclonal anti-TrkB was from BD Biosciences, anti-phospho-S473 Akt, anti-AKT, anti-phospho-ERK1/2, anti-ERK1/2, anti-pY516-TrkB and anti-pY706/707-TrkB were from Cell Signaling, HRP-conjugated streptavidin was from Amersham Biosciences and anti-betaIII-tubulin was from Millipore.

Intraperitoneal Administration to Mice

Adult C57BL/6 male mice were injected i.p. with saline (0.9% NaCl) or LIT-TB001 (dissolved in saline solution) at different doses ranging from 0.1 to 5.0 mg/kg. A volume of 10 μl/g body weight was injected. After 1 hour (unless indicated otherwise), mice were decapitated, blood was collected and brains were rapidly removed on ice. Cortex and hippocampus were subsequently dissected and tissues were rapidly washed in ice-cold PBS and transferred into ice-cold solubilization buffer before homogenization at 4° C. Samples were centrifuged at 10,000×g for 10 min at 4° C. Protein concentrations were determined, equal amounts of proteins were loaded, and western blots were performed as described above.

TrkB Selectivity

The development of Trk canonical (orthosteric) agonists is limited by the lack of selectivity toward the receptor as there is three most common and similar types of Trk receptors: TrkA, TrkB, and TrkC. Each of these receptor has different binding affinity to certain types of neurotrophins. The differences in the signaling initiated by these distinct types of receptors are important for generating diverse biological responses.

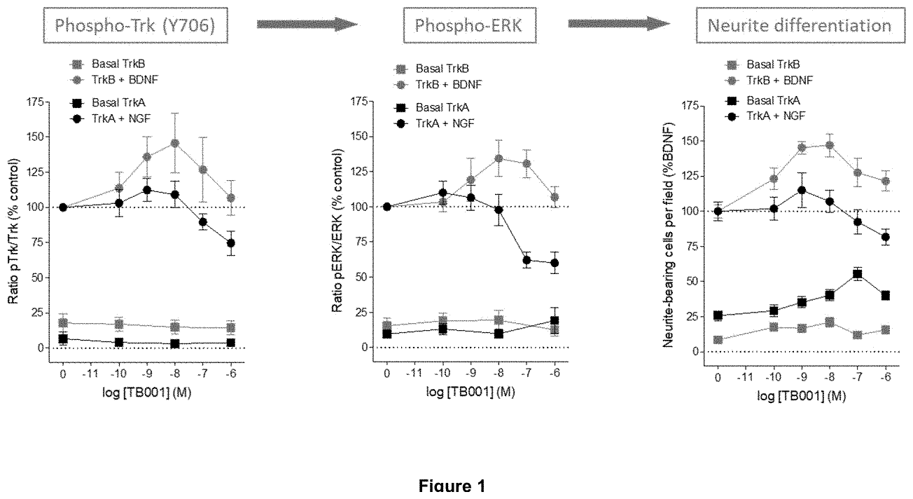

TrkB PAM's could have some advantage in terms of selectivity. Thus the selectivity of LIT-TB001, as a potential TrkB PAM, has been evaluated in vitro toward TrkB (FIG. 1).

Selectivity of LIT-TB001 toward signaling activation and biological function was tested in PC12-TrkB or PC12-TrkA cells, in presence of BDNF (TrkB) or NGF (TrkA). Key experiments were recapitulated in either TrkA or TrkB-expressing cells to test for TB selectivity: Trk phosphorylation, ERK phosphorylation and neurite outgrowth (FIG. 1).

In PC12-TrkA cells, LIT-TB001 did not induce phosphorylation of ERK or TrkA in the presence or absence of NGF. The phosphorylation of ERK and TrkB in PC12-TrkB cells is induced only in the presence of BDNF. The same observations was made at the functional level on neurite outgrowth.

To conclude, LIT-TB001 potentiates BDNF- but not NGF-dependent signaling pathways (pERK and pTrkB) and biological functions (neurite outgrowth). These results show selectivity of the TB compounds toward the Trk family.

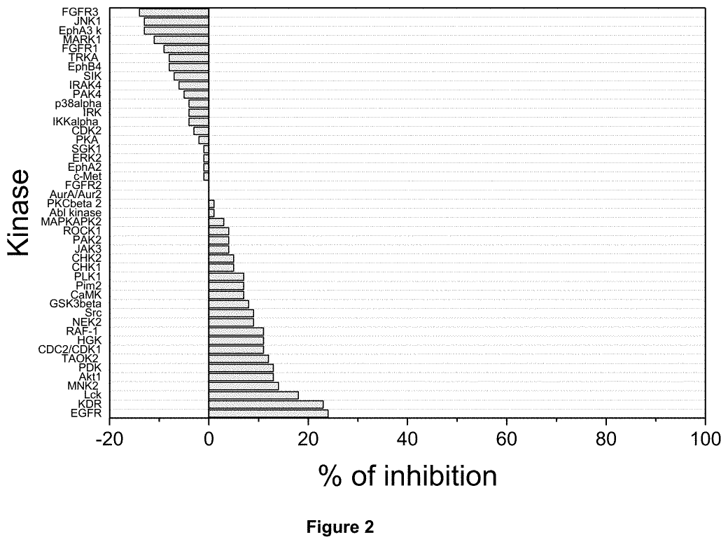

A kinome profile was next performed to test LIT-TB001 selectivity toward other kinases. The kinome profile of 45 kinases has shown a good TrKB selectivity as LIT-TB001 does not activate neither block the catalytic activity of the tested kinases at a concentration of 10 μM (among them TrkA, the most similar to TrkB, confirming our previous results) (FIG. 2).

In Vitro Activity of LIT-TB Derivatives in a TrkB Phosphorylation Assay

In vitro activity of LIT-TB derivatives in a TrkB phosphorylation assay are listed in table 1 below:

| | | PAM |
|---|---|---|
| | | TrkB |
| Nr | LIT-TB Nr | activity^{a} |
| 9a | LIT-TB001 | +++ |
| 9b | LIT-TB002 | ++ |
| 9g | LIT-TB003 | ++ |
| 9f | LIT-TB004 | + |
| 9c | LIT-TB005 | +++ |
| 38 | LIT-TB006 | +++ |
| 24 | LIT-TB012 | ++ |
| 44 | LIT-TB013 | +++ |
| 50 | LIT-TB014 | ++ |
| 54 | LIT-TB015 | +++ |
| 26a | LIT-TB016 | +++ |
| 20a | LIT-TB017 | +++ |
| 20b | LIT-TB018 | +++ |
| 20c | LIT-TB019 | +++ |
| 20d | LIT-TB020 | +++ |
| 19 | LIT-TB021 | + |
| 20e | LIT-TB022 | +++ |
| 20f | LIT-TB023 | + |
| 20g | LIT-TB024 | +++ |
| 20h | LIT-TB025 | + |
| 20i | LIT-TB026 | + |
| 20j | LIT-TB027 | + |
| 20k | LIT-TB028 | + |
| 20l | LIT-TB031 | +++ |
| 20m | LIT-TB032 | +++ |
| 26c | LIT-TB033 | + |
| 26d | LIT-TB034 | + |
| 26e | LIT-TB035 | ++ |
| 20n | LIT-TB040 | ++ |
| 20o | LIT-TB044 | ++ |
| 16p | LIT-TB045 | +++ |
| 20q | LIT-TB046 | + |
| 28a | LIT-TB047 | ++ |
| 28b | LIT-TB048 | +++ |
| 28c | LIT-TB049 | +++ |
| 29b | LIT-TB052 | +++ |
| 29c | LIT-TB053 | ++ |

^{a}In vitro potentation at a PAM concentration of 10 nM of 0.4 nM BDNF-induced TrkB phosphorylation in cortical neurons (+: <20%, ++: 20-35%, +++: >35%). For sake of comparison, a 10-times increase in BDNF concentration (from 0.4 to 4 nM) leads to a potentiation of 55% in this assay.

In Vivo Target Engagement

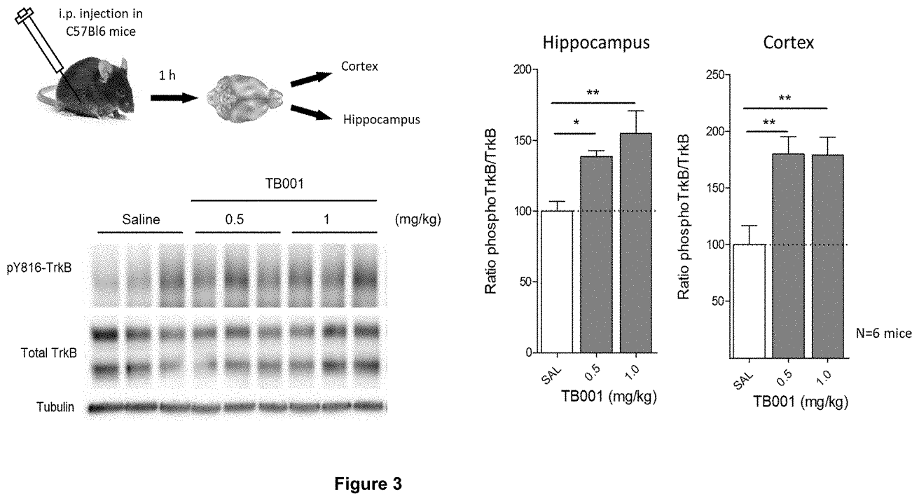

In vivo TrkB engagement by LIT-TB001 was evaluated in the brain of mice after peripheral injection. C57Bl6 male mice received an i.p injection of 0.5 and 1 mg/kg for 1 hour before their brain was carefully removed and their cortex and hippocampus were sub-dissected. BDNF and TrkB are known to play crucial roles in these two regions. The level of TrkB phosphorylation at Tyrosine 816 was analyzed by western blot (FIG. 3). These results unambiguously show that LIT-TB001, at low doses (0.5 and 1 mg/kg, i.p.) efficiently increases TrkB activation in the brain 1 h after systemic administration in mice.

### LIST OF REFERENCES

[1] Vonsattel J. P., DiFiglia M., J. Neuropathol. Exp. Neurol., 57, 369-384, 1998.
[2] Li S-H, Li X-J., Multiple pathways contribute to the pathogenesis of Huntington disease. Molecular Neurodegeneration, 1, 19, 2006.
[3] Harjes P., Wanker E. E., the hunt for huntingtin function: interaction partners tell many different stories. Trends Biochem. Sci, 28, 425-433, 2003.
[4] Schulte J., Littleton J. T., The biological function of the Huntingtin protein and its relevance to Huntington's disease pathology. Current Trends in Neurology, 5, 65-78, 2011.
[5] The Huntington's Disease Collaborative Research Group, A novel gene containing a trinucleotide repeat that is expanded and unstable on Huntington's disease chromosomes, Cell, 72, 971-983, 1993.
[6] Roos RAC, Untington's disease: a clinical review, Orphanet Journal of Rare Diseases, 5, 40, 2010.
[7] Koliatsos V. E., Mocchetti I., Cell Death and Diseases of the Nervous System, Humana Press, Totowa, N.J., 545-591, 1999.
[8] Barbacid M., Structural and functional properties of the Trk family of neurotrophin receptors, Ann. NY Acad. Sci., 766, 442-458, 1995.
[9] Leibrock J., Lottspeich F., Hohn A., Hofer H., Hengerer B., Masiakowski P., Thoenen H., Barde Y. A., Molecular cloning and expression of brain-derived neurotrophic factor, Nature, 341, 149-152, 1989.
[10] Ferrer I., Goutan E., Marin C., Rey M. J., Ribalta T., Brain-derived neurotrophic factor in Huntington disease, Brain Research, 866, 257-261, 2000.
[11] Zuccato C., Ciammola A., Rigamonti D., Leavitt B. R., Goffredo D., Conti L., MacDonald M. E., Friedlander R. M., Silani V., Hayden M. R., Timmusk T., Sipione S., Cattaneo E., Loss of huntingtin-mediated BDNF gene transcription in Huntington's disease. Science, 293, 493-498, 2001.
[12] Gauthier L. R., Charrin B. C., Borrell-Pages M., Dompierre J. P., Rangone H., Cordelibres F. P., De Mey J., MacDonald M. E., Lessmann V., Humbert S., Saudou F. Huntingtin controls neutrophic support and survival of neurons by enhancing BDNF vesicular transport along microtubules. Cell, 118, 127-138, 2004.
[13] Gińes S., Bosch M., Marco S., Gavalds N., Diaz-Hernández M, Lucas J. J., Canals J. M., Alberch J., Reduced expression of the TrKB receptor in Huntington's disease mouse models and in human brain. Eur. J. Neurosci., 23, 649-658, 2006.
[14] Canals J. M., Pineda J. R., Torres-Peraza J. F., Bosch M., Martin-Ibañez R., MuAoz M. T., Mengod G., Ernfors P., Alberch J., Brain-derived neurotrophic factor regulates the onset and severity of motor dysfunction associated with enkephalinergic neuronal degeneration in Huntington's disease. J. Neurosci, 24, 7727-7739, 2004.
[15] Binder D. K., Croll S. D., Gall C. M., Scharfman H. E., BDNF and epilepsy: too much of a good thing? Trends Neurosci., 24, 47-53, 2001.
[16] Green, S. H., Rydel, R. E., Connolly, J. L. Greene, L. A., PC12 cell mutants that possess low- but not high-affinity nerve growth factor receptors neither respond to nor internalize nerve growth factor. J. Cell. Biol. 102, 830-843, 1986.
[17] Cazorla, M., Premont, J., Mann, A., Girard, N., Kellendonk, C., Rognan, D., Identification of a low-molecular weight TrkB antagonist with anxiolytic and antidepressant activity in mice. J. Clin. Invest. 121, 1846-1857, 2011.
[18] Gerencser, J., Panka, G., Nagy, T., Egyed, O., Dorman, G., Urge, L., and Darvas, F., Procedure for the parallel preparation of 3-Imidazo[1,2-a]pyridin-3-yl-propionic acid derivatives involving Meldrum's acid. J. Comb. Chem. 7, 530-538, 2005.

1. A pharmaceutical composition comprising
(a) a LIT-TB compound of formula I: 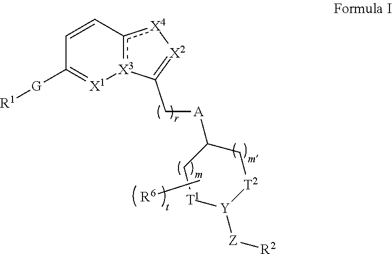
wherein,
R¹ is H, a halogen, a C₁ to C₁₀ saturated or unsaturated, substituted or non-substituted, aliphatic, heteroaliphatic, cyclic, alicyclic, heteroalicyclic, aryl, heteroaryl, alkylaryl or alkylheteroaryl group, or R¹ is a group of formula Ia: 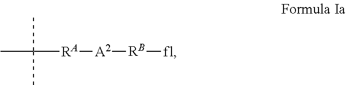
in which,
R^{A} is a linear C₁ to C₁₀ alkyl chain, optionally interrupted by one or more ether or amide functional group,
A² is an amide functional group,
R^{B} is an optionally branched C₁ to C₆ alkyl chain,
fl is a fluorescent group or a non-fluorescent analogue thereof,
G represents a bond or a -G¹-G²- linker in which
G⁴ is a bond or a C1 to C4 substituted or non-substituted alkyl chain, optionally comprising heteroatoms such as N or O and
G² represents a C1 to C10 saturated or unsaturated, substituted or non-substituted, aliphatic, heteroaliphatic, cyclic, alicyclic, heteroalicyclic, aryl, heteroaryl, alkylaryl or alkylheteroaryl group,
X¹ and X², identical or different, independently represents CH or N,
X³ is C or N,
X⁴ is N or NH,
Y represents N or CH,
r is an integer from 1 to 3,
A is an amide or amine functional group,
m is equal to 0, 1 or 2,
m′ is equal to 0, 1 or 2, and m+m′≤3
t is an integer from 0 to 5,
each R⁶ group, identical or different, is H, fluoride, an optionally branched C1 to C6 alkyl chain or a C1 to C6 alkoxy group,
T¹ and T², identical or different, independently represents CH₂, CHR⁶ or C═O,
Z is a bond, H or an optionally branched C1 to C3 alkyl chain, optionally comprising heteroatoms comprising O or N,
R² is null when Z is H or R² is H or a 5- or 6-membered, aromatic or non-aromatic cycle or heterocycle optionally substituted by one or more R⁷ group, each R⁷ group, identical or different, being chosen from H, halide, CN, NO₂, NH₂, CONH₂, an optionally branched C1 to C6 alkyl chain or an optionally branched C1 to C6 alkoxy group, two R⁷ groups being optionally covalently bonded to form a cycle,
or a pharmaceutically acceptable salt thereof, and
(b) a pharmaceutically acceptable excipient or carrier.

2. (canceled)

3. The pharmaceutical composition according to claim 1, wherein X⁴ is N.

4. The pharmaceutical composition according to claim 1, wherein when X⁴ is N, at least one on of X¹, X² and X³ is N.

5. The pharmaceutical composition according to claim 1, wherein R¹ is H, alkyl group, cycloalkyl, aralkyl, heterocycloaryl, or heteroaryl, R1 being optionally substituted.

5. The pharmaceutical composition according to claim 1, wherein R² is H, cycloalkyl, aralkyl, heterocycloaryl, or heteroaryl, R² being optionally substituted by 1, 2 or 3 R⁷ group(s).

6. The pharmaceutical composition according to claim 1, wherein the LIT-TB compound is formula II: 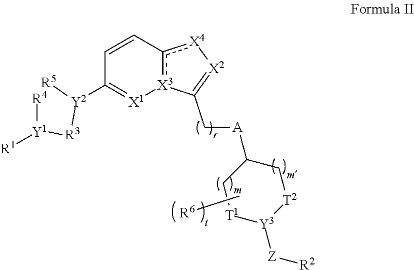
wherein,
X¹, X², X³, X⁴, r, A, m, m′, t, R⁶, T¹, T², Z and R² are defined as above,
Y¹, Y² and Y³, identical or different, independently represents N of CH,
R⁴ and R⁵, identical or different, are independently or an optionally branched C₁ to C₃ alkyl group, optionally comprising heteroatoms comprising O and N, optionally R⁴ and R⁵ may be covalently bonded together to form a cyclic moiety, and
R³ is a linear or branched C₂ to C₆ alkyl chain.

7. The pharmaceutical composition according to claim 1, wherein the LIT-TB compound is formula III: 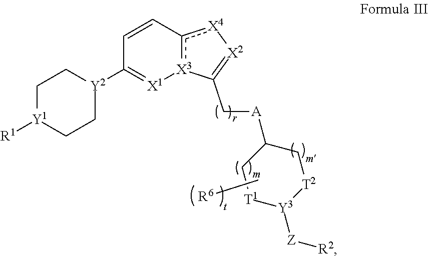
wherein
R¹, X², X³, X⁴, Y¹, Y², Y³, r, A, m, m′, t, R⁶, T¹, T², Z and R² are defined as above.

8. A method for treatment comprising administering a compound of formula I: 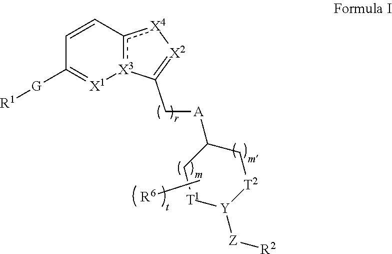
wherein,
R¹ is H, a halogen, a C1 to C10 saturated or unsaturated, substituted or non-substituted, aliphatic, heteroaliphatic, cyclic, alicyclic, heteroalicyclic, aryl, heteroaryl, alkylaryl or alkylheteroaryl group, or R1 is a group of formula Ia: 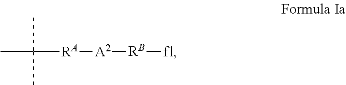
in which,
R^{A} is a linear C1 to C10 alkyl chain, optionally interrupted by one or more ether or amide functional group,
A² is an amide functional group,
R^{B} is an optionally branched C1 to C6 alkyl chain,
fl is a fluorescent group or a non-fluorescent analogue thereof,
G represents a bond or a -G¹-G²- linker in which
G¹ is a bond or a C1 to C4 substituted or non-substituted alkyl chain, optionally comprising heteroatoms such as N or O and
G² represents a C1 to C10 saturated or unsaturated, substituted or non-substituted, aliphatic, heteroaliphatic, cyclic, alicyclic, heteroalicyclic, aryl, heteroaryl, alkylaryl or alkylheteroaryl group,
X¹ and X², identical or different, independently represents CH or N,
X³ is C or N,
X⁴ is N or NH,
Y represents N or CH,
r is an integer from 1 to 3,
A is an amide or amine functional group,
m is equal to 0, 1 or 2,
m′ is equal to 0, 1 or 2, and m+m′≤3
t is an integer from 0 to 5,
each R⁶ group, identical or different, is H, fluoride, an optionally branched C1 to C6 alkyl chain or a C1 to C6 alkoxy group,
T¹ and T², identical or different, independently represents CH₂, CHR⁶ or C═O,
Z is a bond, H or an optionally branched C1 to C3 alkyl chain, optionally comprising heteroatoms comprising O or N, and
R² is null when Z is H or R² is chosen is H or a 5- or 6-membered, aromatic or non-aromatic cycle or heterocycle optionally substituted by one or more R⁷ group, each R⁷ group, identical or different, being chosen from H, halide, CN, NO₂, NH₂, CONH₂, an optionally branched C1 to C6 alkyl chain or an optionally branched C1 to C6 alkoxy group, two R⁷ groups being optionally covalently bonded to form a cycle,
or a pharmaceutically acceptable salt thereof,
to an individual.

9. A method for treating neurodegenerative diseases, metabolic disorders, mood disorders, spinal cord injury, brain stroke or ischemia comprising administering an effective amount of a compound of formula I: 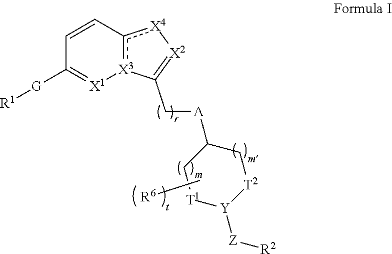
wherein
R¹ is H, a halogen, a C1 to C10 saturated or unsaturated, substituted or non-substituted, aliphatic, heteroaliphatic, cyclic, alicyclic, heteroalicyclic, aryl, heteroaryl, alkylaryl or alkylheteroaryl group, or R¹ is a group of formula Ia: 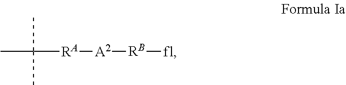
in which,
R^{A} is a linear C1 to C10 alkyl chain, optionally interrupted by one or more ether or amide functional group,
A² is an amide functional group,
R^{B} is an optionally branched C1 to C6 alkyl chain,
fl is a fluorescent group or a non-fluorescent analogue thereof,
G represents a bond or a -G¹-G²- linker in which
G¹ is a bond or a C1 to C4 substituted or non-substituted alkyl chain, optionally comprising heteroatoms such as N or O and
G² represents a C1 to C10 saturated or unsaturated, substituted or non-substituted, aliphatic, heteroaliphatic, cyclic, alicyclic, heteroalicyclic, aryl, heteroaryl, alkylaryl or alkylheteroaryl group,
X¹ and X², identical or different, independently represents CH or N,
X³ is C or N,
X⁴ is N or NH,
Y represents N or CH,
r is an integer from 1 to 3,
A is an amide or amine functional group,
m is equal to 0, 1 or 2,
m′ is equal to 0, 1 or 2, and m+m′≤3
t is an integer from 0 to 5,
each R⁶ group, identical or different, is H, fluoride, an optionally branched C1 to C6 alkyl chain and or a C1 to C6 alkoxy group,
T⁴ and T², identical or different, independently represents CH₂, CHR⁶ or C═O,
Z is a bond, H or an optionally branched C1 to C3 alkyl chain, optionally comprising heteroatoms comprising O or N,
R² is null when Z is H or R² is H or a 5- or 6-membered, aromatic or non-aromatic cycle or heterocycle optionally substituted by one or more R⁷ group, each R⁷ group, identical or different, being chosen from H, halide, CN, NO₂, NH₂, CONH₂, an optionally branched C1 to C6 alkyl chain or an optionally branched C1 to C6 alkoxy group, two R⁷ groups being optionally covalently bonded to form a cycle,
or a pharmaceutically acceptable salt thereof,
to an individual in need thereof.

10. The compound of formula I: 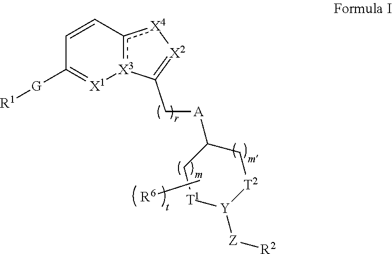
wherein,
R¹ is H, a halogen, a C1 to C10 saturated or unsaturated, substituted or non-substituted, aliphatic, heteroaliphatic, cyclic, alicyclic, heteroalicyclic, aryl, heteroaryl, alkylaryl or alkylheteroaryl group, or R1 is a group of formula Ia: 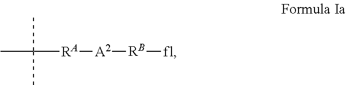
in which,
R^{A} is a linear C1 to C10 alkyl chain, optionally interrupted by one or more ether or amide functional group,
A² is an amide functional group,
R^{B} is an optionally branched C1 to C6 alkyl chain,
fl is a fluorescent group or a non-fluorescent analogue thereof,
G represents a bond or a -G¹-G²- linker in which
G¹ is a bond or a C1 to C4 substituted or non-substituted alkyl chain, optionally comprising heteroatoms such as N or O and
G² represents a C1 to C10 saturated or unsaturated, substituted or non-substituted, aliphatic, heteroaliphatic, cyclic, alicyclic, heteroalicyclic, aryl, heteroaryl, alkylaryl or alkylheteroaryl group,
X¹ and X², identical or different, independently represents CH or N,
X³ is C or N,
X⁴ is N or NH,
Y represents N or CH,
r is an integer from 1 to 3,
A is an amide or amine functional group,
m is equal to 0, 1 or 2,
m′ is equal to 0, 1 or 2, and m+m′≤3
t is an integer from 0 to 5,
each R⁶ group, identical or different, is H, fluoride, an optionally branched C1 to C6 alkyl chain or a C1 to C6 alkoxy group,
T¹ and T², identical or different, independently represents CH₂, CHR⁶ or C═O,
Z is a bond, H and or an optionally branched C1 to C3 alkyl chain, optionally comprising heteroatoms comprising O or N,
R² is null when Z is H or R² is H or a 5- or 6-membered, aromatic or non-aromatic cycle or heterocycle optionally substituted by one or more R⁷ group, each R⁷ group, identical or different, being chosen from H, halide, CN, NO₂, NH₂, CONH₂, an optionally branched C1 to C6 alkyl chain or an optionally branched C1 to C6 alkoxy group, two R⁷ groups being optionally covalently bonded to form a cycle,
or a pharmaceutically acceptable salt thereof,
with the exclusion of:
N-(1-benzyl-4-piperidyl)-3-[6-(4-methylpiperazin-1-yl)-[1,2,4]triazolo[4,3-b]pyridazin-3-yl]propanamide and
N-(1-benzyl-4-piperidyl)-3-[6-(1-piperidyl)-[1,2,4]triazolo[4,3-b]pyridazin-3-yl]propanamide.

11. The pharmaceutical composition of claim 1, wherein A is C(O)NH, NHC(O) or NH.

12. The pharmaceutical composition according to claim 1, wherein, when X⁴ is NH, X³ is C.

13. The pharmaceutical composition according to claim 1, wherein X³ is N and X⁴ is N.

14. The method according to claim 8, wherein A is C(O)NH, NHC(O) or NH.

15. The method according to claim 9, wherein A is C(O)NH, NHC(O) or NH.

16. The compound according to claim 10, wherein A is C(O)NH, NHC(O) or NH.
